(19) United States
(12) Patent Application Publication
(10) Pub. No.: US 2007/0219125 A1
(43) Pub. Date: Sep. 20, 2007

(54) Novel thrombospondin-1 polynucleotides encoding variant thrombospondin-1 polypeptides and methods using same

(76) Inventors: Gad S. Cojocaru, Ramat-HaSharon (IL); Zurit Levine, Herzliya (IL); Michal Ayalon-Soffer, Ramat-HaSharon (IL); Amir Toporik, Pardes Hana (IL); Sarah Pollock, Tel-Aviv (IL); Galit Rotman, Herzliya (IL); Merav Beiman, Nes Ziona (IL)

Correspondence Address: STAAS & HALSEY LLP, SUITE 700, 1201 NEW YORK AVENUE, N.W., WASHINGTON, DC, 20005, US

(21) Appl. No.: 11/709,841

(22) Filed: Feb. 23, 2007

Related U.S. Application Data
(63) Continuation-in-part of application No. 10/130,138, filed on Jul. 25, 2002.
(63) Continuation-in-part of application No. 11/443,428, filed on May 31, 2006.
(60) Provisional application No. 60/775,778, filed on Feb. 23, 2006.
(60) Provisional application No. 60/815,561, filed on Jun. 22, 2006.

(30) Foreign Application Priority Data
Nov. 17, 1999 (IL) 132978
Dec. 10, 1999 (IL) 133455

(51) Int. Cl. A61K 38/36; C07H 21/04; C07K 14/745; C07K 16/36; C12N 5/10

(52) U.S. Cl. 514/8; 435/325; 530/381; 530/387.9; 536/24.1

(57) ABSTRACT

Novel polypeptides and polynucleotides encoding same are provided. Also provided methods and phamaceutical compositions which can be used to treat various disorders such as cancer and retinopathies, using the polypeptides and polynucleotides of the present invention.

### CROSS-REFERENCE TO RELATED APPLICATIONS

This application is a continuation-in-part of U.S. Ser. No. 10/130,138, filed May 16, 2002, now pending, which is the national phase under 35 U.S.C. Section 371 of PCT International Application No. PCT/IL00/00766 which has an International filing date of Nov. 17, 2000, which designated the United States of America, which claims the benefit of Israeli Patent Application No. 132978 filed Nov. 17, 1999 and Israeli Patent Application No. 133455 filed Dec. 10, 1999, and claims priority under U.S. Provisional Application No. 60/775,778 filed on Feb. 23, 2006, and U.S. Provisional Application No. 60/815,561 filed on Jun. 22, 2006, and is a continuation-in-part of U.S. Ser. No. 11/443,428 filed on May 31, 2006, the disclosures of which are incorporated herein by reference.

### FIELD OF THE INVENTION

The present invention relates to novel thrombospondin-1 (TSP-1) variant polypeptides and polynucleotides encoding same and to therapeutic methods and compositions utilizing same.

### BACKGROUND OF THE INVENTION

Thrombospondins are a family of calcium-binding multifunctional glycoproteins that are secreted by various cell types and are developmentally regulated components of the extracellular matrix (Bornstein, P., FASEB J., 6:3290-3299, 1992; Bornstein, P., J. Cell Biol., 130:503-506, 1995). The functions of the members of this family include modulating cell attachment, migration and proliferation, and angiogenesis. The thrombospondin family comprises a group of five members characterized by a specific modular organization. Two of these proteins, TSP-1 and TSP-2, feature the so-called “thrombospondin repeats” (TSR). TSP-1 and TSP-2 are the most well known members of this family. TSP-1 (thrombospondin-1) is a 450-kDa glycoprotein that is stored in the alpha-granules of platelets and is secreted by a number of cell types. TSP-1 features three identical 150-kDa monomers connected by disulfide bridges. The primary anti-angiogenic activity of TSP-1 has been localized to its procollagen domain and type-1 repeat (TSR) sequences. The three TSRs of TSP-1 (3TSR) comprise an 18-kD peptide, which binds to CD36, an important receptor for TSP-1 signaling under many experimental conditions. The 3TSR domain has been shown to inhibit VEGF-induced migration in endothelial cells and small peptides derived from each of the TSR repeats are independently able to block endothelial cell migration in vitro and neovascularization in vivo.

TSP-1 is known as an angiogenic modulator, and is also involved in thrombosis, fibrinolysis, wound healing, inflammation and tumor cell metastasis. Fragments of TSP-1 were shown to have anti-angiogenic effects, as was the entire molecule under some circumstances (for a review see Sargiannidou et al, Seminars in Thrombosis and Hemostasis, Vol 30, Number 1, 2004, pp 127-136). However, under other circumstances the whole TSP-1 molecule was shown to be pro-angiogenic. TSP-1 also blocks multiple pro-angiogenic growth factors including VEGF, bFGF, and IL-8.

TSP-1 is believed to play other roles in the formation of metastases. For example, it is believed to accelerate or enable the formation of blood clots, which may also feature metastatic cells. Normally only present in very low quantities in plasma, upon blood coagulation and activation of platelets, TSP-1 is released into serum and is incorporated into fibrin clots. Furthermore, TSP-1 mediates the adhesion of such clots to blood vessel walls, thereby enabling the malignant cells to escape into other organs. As part of this activity, TSP-1 also mediates adhesion to the basement membrane, which is a necessary prerequisite for malignant cells to escape from the blood vessel. On the other hand, TSP-1 has anti-cancer functions such as activation of latent transforming growth factor-b (TGF-b) and tumor growth inhibition.

The role of TSP-1 in cancer depends upon the specific type of cancer. For example, increased expression of TSP-1 is shown in breast cancer and colon carcinoma. In other types of cancer, such as bladder and ovarian carcinoma, later stages of the disease actually show decreased expression of TSP-1 (Sargiannidou et al, Seminars in Thrombosis and Hemostasis, Vol 30, Number 1, 2004, pp 127-136). Increased expression of TSP-1 does not necessarily result in an increase in angiogenesis. Rather, increased TSP-1 levels may promote metastases in other ways as described above.

The effects of TSP-1 can be mediated through a number of different receptors, including integrin, CD47, CD36 (also known as GP 88, GP IIIb or GP IV), and HSPG (heparan sulfate proteoglycans) (see Sid et al, Critical Reviews in Oncology/Hematology, Vol 49, 2004, pp 245-258, for a review). TSP-1 also modulates various activities through modulation of the activity of matrix metalloproteinase 9 (MMP9).

Various therapies based on TSP-1 have been proposed. For example, ABT-510 is a subcutaneously (SC) administered nonapeptide thrombospondin analogue in phase 2 clinical development by Abbot Laboratories for treatment of advanced malignancies, including sarcoma, lymphoma (NHL), lung and kidney cancer. ABT-510 blocks the actions of multiple pro-angiogenic growth factors known to play a role in cancer related blood vessel growth, such as VEGF, bFGF, HGF, and IL-8 (Haviv et al (2005), J. Med. Chem. 48, 2838-2846; Baker et al (2005) J. Clin. Oncol. 23, 9013). Another example are the products being developed by TSP Pharma, which target TSP-1 Binding Protein (Angiocidin), found on the surface of cancer cells. One product example is Cevastat. Cevastat is a peptide with potent binding to Angiocidin that inhibits the protein's function and is being developed for the treatment of multiple cancers (including colon, lung, prostate and pancreas). Cevastat is also used as a targeting agent to deliver a therapeutic dose of radiation to the tumor cells (“Cevastat-Y”). Additionally, TSP Pharma is developing a series of monoclonal antibodies that bind to Angiocidin resulting in a reduction in tumor growth by selectively disrupting the tumor's blood supply. Additional examples are Angiocidin, a soluble thrombospondin receptor drug/TSP-1 binding protein, developed by InKine Pharmaceuticals; and an antisense oligonucleotide directed against thrombospondin, for the treatment of squamous cell carcinoma, developed by Genta.

Major progress has been made over the past few years in targeting angiogenesis for human therapy. The outcomes of several clinical trials have validated the notion that angiogenesis is an important target for cancer and other diseases. TSP-1 derived anti-angiogenic agents are considered as novel and promising anti-angiogenic therapies, particularly since they derive from a natural anti-angiogenic protein, as opposed to current approaches that antagonize pro-angiogenic factors, and are thus prone to undesirable side effects.

Targeted cancer therapy, including anti-angiogenic strategies, appears more efficient as combination therapy than as monotherapy. Vast preclinical evidence indicates that combining anti-angiogenic agents with conventional cytotoxic agents or radiation therapy results in additive or even synergistic anti-tumor effects (Gasparini et al 2005, J. Clin. Oncol. 23: 1295-1311). In agreement with that, the results of recent clinical trials indicate that anti-angiogenic drugs may render cancer cells more sensitive to cytotoxic chemotherapy or radiotherapy without substantially increasing toxicity to normal cells (Kerbel, 2006, Science 312:1171-1175). There are several possible mechanisms by which the sensitizing effect of anti-angiogenic agents make take place, such as normalizing tumor vasculature, preventing rapid tumor cell repopulation and augmenting the antivascular effects cytotoxic agents. Combinatorial therapies with anti-angiogenic agents are not limited to those including cytotoxic chemotherapy, but may take place with other anti-angiogenic agents and/or with tumor-targeted therapies.

Ocular neovascularization and vascular leakage are a major cause of visual loss in a number of human ocular diseases due to retinal angiogenesis, such as diabetic retinopathy, retinopathy of prematurity, and age-related macular degeneration. Several anti-angiogenic strategies are being explored in clinical trials for these diseases.

Although various therapies based on TSP-1 have been proposed, particularly for treatment of cancer, these proposed therapies have generally avoided use of the entire TSP-1 molecule, because the whole molecule has various different functions, some of which are anti-cancer while others may enable the spread of metastases. One problem with selecting therapies that only incorporate or bind to a part of the TSP-1 molecule is that it is difficult to know which therapy will provide the most efficient combination of anti-cancer functionality in vivo.

### SUMMARY OF THE INVENTION

In view of its critical role in angiogenesis and oncogenesis, there is an unmet need to develop therapies based on TSP-1. The background art does not teach or suggest variants of TSP-1 protein. The background art also does not teach or suggest variants of TSP-1 and protein that are useful as therapeutic proteins or peptides for a range of cluster-related clinical conditions and/or variant-treatable diseases.

The present invention overcomes these deficiencies of the background art by providing novel splice variants of TSP-1 therapeutic protein and derivatives thereof, which may optionally be used as therapeutic proteins or peptides. Specifically, the present invention provides TSP-1 therapeutic protein and derivatives thereof having anti-angiogenic activity.

According to certain aspects of the present invention, the TSP-1 therapeutic protein variants of the present invention comprise an amino acid sequence as described in TSP-1_{—}1112 (SEQ ID NO: 51, 56); TSP-1_{—}685 (SEQ ID NO: 50, 58); TSP-1_{—}555 (SEQ ID NO: 52, 60), TSP-1_{—}578 (SEQ ID NO:48) and TSP-1_{—}804 (SEQ ID NO:49). According to a further aspect of the present invention, there are nucleic acid sequences encoding the TSP-I therapeutic protein variants of the present invention, represented herein by SEQ ID NO:5 for TSP-1_{—}1112; SEQ ID NO:4 for TSP-1_{—}685; SEQ ID NO:2 for TSP-1_{—}555, SEQ ID NO:1 for TSP-1_{—}578 and SEQ ID NO:3 for TSP-1_{—}804. The corresponding optimized nucleic acid sequences are represented herein by SEQ ID NO:55 for TSP-1_{—}1112; SEQ ID NO:57 for TSP-1_{—}685; and SEQ ID NO:59 for TSP-1_{—}555.

Optionally and preferably, these therapeutic protein variants and derived peptides of the present invention can be modified to form synthetically modified variants according to the present invention, wherein modified variants include but are not limited to fusion proteins (including but not limited to fusion with an Fc fragment of Ig) and/or linked to expression tags, including but not limited to Strep-His tag, and/or chemical modifications, including but not limited to pegylation.

Preferably, these therapeutic proteins and derived peptides are useful as therapeutic proteins or peptides for diseases including but not limited to cluster-related variant-treatable diseases.

Surprisingly, as uncovered by the present inventors, novel naturally occurring splice variants of TSP-1 gene products according to the present invention can be used in the therapy of a wide range of variant-detectable diseases and variant-treatable diseases, which are “TSP-1-related diseases”. These splice variants of the present invention can be used as valuable therapeutic tools in the treatment of “TSP-1-related diseases”.

As meant herein, “TSP-1-related disease(s)” (also named “variant treatable disease(s)”) refers to a disease in which TSP-1 activity and/or expression modulates disease onset and/or progression, such that treating the disease may involve influencing TSP-1 activity and/or expression. Examples of TSP-1-related diseases include, but are not limited to, cancer, such as, primary cancer and tumor cell metastasis. “TSP-1-related disease(s)” refers preferably to diseases in which anti-angiogenic activity plays a favorable role, including but not limited to, diseases having abnormal quality and/or quantity of vascularization as a characteristic feature, such as cancer, including but not limited to prostate cancer, renal cancer, cervical carcinomas, breast cancer, colon and colorectal cancer, pancreatic cancer, ovarian cancer, bladder cancer, lung cancer, melanoma, brain cancer, glioblastomas, soft tissue sarcomas, head-and-neck cancer, lymphomas, and other tumors and metastatic cancers. Other examples of TSP-1-related diseases include, but are not limited to, diseases that involve treatment of retinal angiogenesis, in human ocular diseases, such as diabetic retinopathy, retinopathy of prematurity, and age-related macular degeneration. Additional examples of TSP-1-related diseases include, but are not limited to, wound healing and inflammation, such as rheumatoid arthritis.

TSP-1 variants of the present invention can be used as carriers or targetors of cytotoxic drugs, and can be useful as anticancer therapeutic agents. Thus, according to an optional embodiment of the present invention, the variants of the present invention can optionally be conjugated to a bioactive moiety, preferably selected from the group consisting of but not limited to a cytotoxic compound, a cytostatic compound, an antisense compound, an anti-viral agent, a specific antibody, an imaging agent and a biodegradable carrier.

Thus, the present invention envisages treatment of the above-mentioned diseases by the provision of polynucleotide or polypeptide sequences of this aspect of the present invention, which are capable of upregulating the level of the polypeptides of the present invention in a subject in need thereof, as is further described hereinbelow. Such polynucleotide or polypeptide sequences of this aspect of the present invention and administration thereof are further described hereinbelow. This includes the use of the TSP-1 variants of the invention as monotherapy for cancer, or in combination therapy with any of various other cytotoxic agents, or anti-angiogenic and/or anti-tumor agents.

As used herein the phrase “disease” includes any type of pathology and/or damage, including both chronic and acute damage, as well as a progress from acute to chronic damage.

As used herein, the term “level” refers to expression levels of RNA and/or protein or to DNA copy number of a marker of the present invention.

According to certain embodiments of the present invention, the invention provides isolated nucleic acid sequences of TSP-1 variants comprising the sequences described herein.

According to other embodiments, the present invention provides amino acid sequences of TSP-1 variants comprising the sequences described herein.

According to other embodiments, the present invention provides head, tail, bridge or edge sequence described herein.

According to other embodiments, the present invention provides an antibody capable of specifically binding to an epitope of an amino acid sequence of TSP-1 variants comprising the sequences described herein and/or to an epitope of head, tail, bridge, edge or insertion sequence described herein.

According to yet further embodiments, the present invention provides said antibody, wherein said antibody is capable of differentiating between a splice variant having said epitope and a corresponding known protein.

According to other embodiments, the invention provides a pharmaceutical composition comprising as an active ingredient any of the above nucleic acid sequences or a fragment thereof, or any of the above amino acid sequences or a fragment thereof.

According to other embodiments, the present invention provides a method for treating a variant-treatable disease, comprising administering a therapeutic protein, variant peptide, protein, nucleic acid sequence, antisense and/or antibody to a subject in need of treatment thereof.

The variant-treatable disease is preferably a cluster TSP-1-treatable disease and is selected from the group consisting of cancerous diseases, including but not limited to primary cancer and tumor cell metastasis. The cluster TSP-1-treatable disease is optionally and preferably selected from the group consisting of diseases in which anti-angiogenic activity plays a favorable role, including but not limited to, diseases having abnormal quality and/or quantity of vascularization as a characteristic feature, such as cancer for example, including but not limited to breast cancer, colon cancer, pancreatic cancer, ovarian cancer, bladder cancer, lung cancer, melanoma, brain cancer, and other solid tumors and metastatic cancers. Alternatively, the cluster TSP-1-treatable disease is selected from the group consisting of inflammatory disorders including but not limited to, wound healing and inflammation, such as rheumatoid arthritis.

According to optional but preferred embodiments of the present invention, there is provided a nucleic acid construct comprising the isolated polynucleotide as described herein. Preferably, the nucleic acid construct further comprises a promoter for regulating transcription of the isolated polynucleotide in sense or antisense orientation. Also preferably, the nucleic acid construct further comprises positive and negative selection markers for selecting for homologous recombination events.

According to other optional but preferred embodiments of the present invention, there is provided a host cell comprising the nucleic acid construct as described herein.

According to preferred embodiments of the present invention, there is provided a pharmaceutical composition comprising a therapeutically effective amount of a polypeptide as described herein and a pharmaceutically acceptable carrier or diluent.

According to preferred embodiments of the present invention, there is provided a method of treating a variant-related disease in a subject, the method comprising upregulating in the subject expression of a polypeptide as described herein, thereby treating the variant-related disease in a subject. Optionally, upregulating expression of said polypeptide is effected by:

(i) administering said polypeptide to the subject; and/or

(ii) administering an expressible polynucleotide encoding said polypeptide to the subject.

According to preferred embodiments of the present invention, there is provided, as listed below, optional but preferred embodiments (although provided as a list, this is for the sake of convenience only and is not intended to indicate a closed list or to otherwise be limiting in any way):

According to preferred embodiments of the present invention, there is provided an isolated polynucleotide comprising a transcript selected from the group consisting of HUMTHROM_{—}1_T12 (SEQ ID NO:1), HUMTHROM_{—}1_T14 (SEQ ID NO:2), HUMTHROM_{—}1_T15 (SEQ ID NO:3), HUMTHROM_{—}1_T17 (SEQ ID NO:4), HUMTHROM_{—}1_T32 (SEQ ID NO:5), or a polynucleotide at least about 95% homologous thereto.

According to preferred embodiments of the present invention, there is provided an isolated polypeptide comprising a protein variant selected from the group consisting of HUMTHROM_{—}1_P8 (SEQ ID NO:48), HUMTHROM_{—}1_P10 (SEQ ID NO:49), HUMTHROM_{—}1_P12 (SEQ ID NO:50), HUMTHROM_{—}1_P22 (SEQ ID NO:51), HUMTHROM_{—}1_P27 (SEQ ID NO:52), or a polypeptide at least about 95% homologous thereto.

According to preferred embodiments of the present invention, there is provided an isolated chimeric polypeptide encoding for HUMTHROM_{—}1_PO (SEQ ID NO:49), comprising a first amino acid sequence being at least 90% homologous to amino acids 1-751 of TSP-1_HUMAN_V1 (SEQ ID NO:47), which also corresponds to amino acids 1-751 of HUMTHROM_{—}1_P10 (SEQ ID NO:49), and a second amino acid sequence being at least 70%, optionally at least 80%, preferably at least 85%, more preferably at least 90% and most preferably at least 95% homologous to a polypeptide having the sequence VKTVFYPFFIFSVQQQPETLWDSRKLHGYSKKYTKSIHRIIRNYSLCSSSLRM corresponding to amino acids 752-804 of HUMTHROM_{—}1_P10 (SEQ ID NO:49), wherein said first amino acid sequence and second amino acid sequence are contiguous and in a sequential order.

According to preferred embodiments of the present invention, there is provided an isolated polypeptide encoding for an edge portion of HUMTHROM_{—}1_P10 (SEQ ID NO:49), comprising an amino acid sequence being at least 70%, optionally at least about 80%, preferably at least about 85%, more preferably at least about 90% and most preferably at least about 95% homologous to the sequence VKTVFYPFFIFSVQQQPETLWDSRKLHGYSKKYTKSIHRIIRNYSLCSSSLRM of HUMTHROM_{—}1_P10 (SEQ ID NO:49).

According to preferred embodiments of the present invention, there is provided an isolated chimeric polypeptide encoding for HUMTHROM_{—}1_P12 (SEQ ID NO:50), comprising a first amino acid sequence being at least 90% homologous to amino acids 1-643 of TSP-1_HUMAN_VI (SEQ ID NO:47), which also corresponds to amino acids 1-643 of HUMTHROM_{—}1_P12 (SEQ ID NO:50), and a second amino acid sequence being at least 70%, optionally at least 80%, preferably at least 85%, more preferably at least 90% and most preferably at least 95% homologous to a polypeptide having the sequence QSTRRVNQRTGELSLTKITGSGRNVISYPSPKKKGRGDECTV corresponding to amino acids 644-685 of HUMTHROM_{—}1_P12 (SEQ ID NO:50), wherein said first amino acid sequence and second amino acid sequence are contiguous and in a sequential order.

According to preferred embodiments of the present invention, there is provided an isolated polypeptide encoding for an edge portion of HUMTHROM_{—}1_P12 (SEQ ID NO:50), comprising an amino acid sequence being at least 70%, optionally at least about 80%, preferably at least about 85%, more preferably at least about 90% and most preferably at least about 95% homologous to the sequence QSTRRVNQRTGELSLTKITGSGRNVISYPSPKKKGRGDECTV of HUMTHROM_{—}1_P12 (SEQ ID NO:50).

According to preferred embodiments of the present invention, there is provided an isolated chimeric polypeptide encoding for HUMTHROM_{—}1_P22 (SEQ ID NO:51), comprising a first amino acid sequence being at least 90% homologous to amino acids 1-490 of TSP-1_HUMAN_VI (SEQ ID NO:47), which also corresponds to amino acids 1-490 of HUMTHROM_{—}1_P22 (SEQ ID NO:51), a second bridging amino acid sequence comprising of N, and a third amino acid sequence being at least 90% homologous to amino acids 550-1170 of TSP-1_HUMAN_V1 (SEQ ID NO:47), which also corresponds to amino acids 492-1112 of HUMTHROM_{—}1_P22 (SEQ ID NO:51), wherein said first amino acid sequence, second amino acid sequence and third amino acid sequence are contiguous and in a sequential order.

According to preferred embodiments of the present invention, there is provided an isolated polypeptide encoding for an edge portion of HUMTHROM_{—}1_P22 (SEQ ID NO:51), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least three amino acids comprise PNG having a structure as follows (numbering according to HUMTHROM_{—}1_P22 (SEQ ID NO:51)): a sequence starting from any of amino acid numbers 490-x to 490; and ending at any of amino acid numbers 492+((n−2)−x), in which x varies from 0 to n−2.

According to preferred embodiments of the present invention, there is provided an antibody capable of specifically binding to an epitope of an amino acid sequence as described herein.

According to preferred embodiments of the present invention, there is provided an antibody capable of specifically binding to an epitope of an amino acid sequence as described above, optionally wherein said amino acid sequence corresponds to a bridge, edge portion, tail, or head as in any of the previous claims, also optionally wherein said antibody is capable of differentiating between a splice variant having said epitope and a corresponding known protein.

According to preferred embodiments of the present invention, there is provided a method for treating a variant-treatable disease, comprising administering a therapeutic protein, variant peptide, protein, nucleic acid sequence, antisense and/or antibody to a subject in need of treatment thereof. Optionally, the variant-treatable disease is cluster HUMTHROM-treatable disease and is selected from the group consisting of cancer, such as, primary cancer and tumor cell metastasis. Alternatively or additionally, the cluster TSP-1-treatable disease is selected from the group consisting of diseases in which anti-angiogenic activity plays a favorable role. Such diseases include, but are not limited to, diseases having abnormal quality and/or quantity of vascularization as a characteristic feature, such cancer, including but not limited to breast cancer, colon cancer, pancreatic cancer, ovarian cancer, bladder cancer, lung cancer, melanoma, brain cancer, and other solid tumors and metastatic cancers. Alternatively or additionally, the cluster TSP-1-treatable disease is selected from the group consisting of inflammatory disorders including but not limited to, wound healing and inflammation, such as rheumatoid arthritis.

According to optional but preferred embodiments of the present invention, there is provided a nucleic acid construct comprising the isolated polynucleotide as described herein. Preferably, the nucleic acid construct further comprises a promoter for regulating transcription of the isolated polynucleotide in sense or antisense orientation. Also preferably, the nucleic acid construct further comprises positive and negative selection markers for selecting for homologous recombination events.

According to other optional but preferred embodiments of the present invention, there is provided a host cell comprising the nucleic acid construct as described herein.

According to preferred embodiments of the present invention, there is provided a pharmaceutical composition comprising a therapeutically effective amount of a polypeptide as described herein and a pharmaceutically acceptable carrier or diluent.

According to preferred embodiments of the present invention, there is provided a method of treating a variant-related disease in a subject, the method comprising upregulating in the subject expression of a polypeptide as described herein, thereby treating the variant-related disease in a subject. Optionally, upregulating expression of said polypeptide is effected by:

(i) administering said polypeptide to the subject; and/or

(ii) administering an expressible polynucleotide encoding said polypeptide to the subject.

Alternatively and optionally, the kit comprises an antibody according to any of the above claims (optionally and preferably, the kit further comprises at least one reagent for performing an ELISA or a Western blot.

All nucleic acid sequences and/or amino acid sequences shown herein as embodiments of the present invention relate to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that oligonucleotide and polynucleotide, or peptide and polypeptide, may optionally be used interchangeably.

Information given in the text with regard to cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.htm1) for transmembrane region prediction; (iii) signalp_hmm and (iv) signalp_nn (both from Center for Biological Sequence Analysis, Technical University of Denmark DTU, http://www.cbs.dtu.dk/services/SignalP/background/prediction.php) for signal peptide prediction. The terms “signalp_hmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) Evolution of multicellularity in metazoa: comparative analysis of the subcellular localization of proteins in Saccharomyces, Drosophila and Caenorhabditis. Cell Biology International 2004;28(3):171-8. ], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T ->C”, for example, means that the SNP results in a change at the position given in the table from T to C. Similarly, “M ->Q”, for example, means that the SNP has caused a change in the corresponding amino acid sequence, from methionine (M) to glutamine (Q). If, in place of a letter at the right hand side for the nucleotide sequence SNP, there is a space, it indicates that a frameshift has occurred. A frameshift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTld=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. For each given SNP, it was determined whether it was previously known by using dbSNP build 122 from NCBI, released on Aug. 13, 2004.

Information given in the text with regard to the Homology to the wild type was determined by Smith-Waterman version 5.1.2 Using Special (non default) parameters as follows:
model=sw.model
GAPEXT=0
GAPOP=100.0
MATRIX=blosum 100

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In anticipation of the grant of the Petition, and which, if not granted, will be amended accordingly, the following paragraph is added beginning at line 4 of page 15:

The patent or application file contains at least one drawing executed in color. Copies of this patent or patent application publication with color drawing(s) will be provided by the U.S. Patent and Trademark Office upon request and payment of the necessary fee. These and/or other aspects and advantages of the invention will become apparent and more readily appreciated from the following description of the embodiments, taken in conjunction with the accompanying drawings, of which: In the drawings:

FIG. 1 presents TSP-1 mRNA and protein schematic structure. TSP-1 variants ofthe present invention, TSP-1_{—}1112 (SEQ ID NO:5, 51); TSP-1_{—}685 (SEQ ID NO:4, 50); TSP-1_{—}555 (SEQ ID NO:2, 52), TSP-1_{—}578 (SEQ ID NO:1, 48) and TSP-1_{—}804 (SEQ ID NO:3, 49) are shown as compared to previously derscribed 3TSR fragment (P173) and the known WT 1170 variant. Exons are represented by orange boxes, while introns are represented by two headed arrows. Proteins are shown in yellow boxes. The unique regions are colored green. The heparin binding domain and the TSR domains are indicated

FIG. 2 shows a schematic map of the polynucleotide coding for TSP-1_{—}555 in the pIRESpuro3 expression vector.

FIG. 3 shows the optimized nucleotide sequences of of all the TSP-1 variants, prepared for cloning in the expression vector plRESpuro3, and their respective protein sequences. The relevant ORFs (open reading frames) including the tag sequences are shown in bold; StrepHis tag sequences are underlined. FIG. 3A demonstrates the nucleic acid (SEQ ID NO:53) and the amino acid sequence (SEQ ID NO:54) of TSP-1-1170; FIG. 3B demonstrates the nucleic acid (SEQ ID NO:55 and the amino acid (SEQ ID NO:56) sequence of TSP-1-1112; FIG. 3C demonstrates the nucleic acid (SEQ ID NO:57) and the amino acid (SEQ ID NO:58) sequence of TSP-1-685; FIG. 3D demonstrates the nucleic acid (SEQ ID NO:59) and the amino acid (SEQ ID NO:60) sequence of TSP-1-555; FIG. 3E demonstrates the nucleic acid (SEQ ID NO:61) and the amino acid (SEQ ID NO:62) sequence of TSP-1-173.

FIG. 4 shows the Western blot results, demonstrating stable TSP-1 expression. FIG. 4A lane 5 represents the expression of TSP-1_{—}173 (3TSR) (SEQ ID NO:62); lane 7 represents TSP-1_{—}555 (SEQ ID NO:60); lane 1 represents molecular weight marker (Rainbow Amersham RPN800); lane 2 represents mock pIRESpuro3; and lane 8 represents Strep-His control (˜100 ng). FIG. 4B lane 2 represents the expression of TSP-1_{—}685 (SEQ ID NO:58); lane 1 represents molecular weight marker (Rainbow Amersham RPN800); and lane 8 represents Strep-His control (˜100 ng). FIG. 4C lane 13 represents the expression of TSP-1_{—}1170 (SEQ ID NO:54); lane 12 represents molecular weight marker (Rainbow Amersham RPN800); lane 22 represents Strep-His control (˜100 ng). FIG. 4D lane 10 represents the expression of TSP-1_{—}1112 (SEQ ID NO:56); lane 1 represents molecular weight marker (Rainbow Amersham RPN800); and lane 12 represents Strep-His control (˜100 ng).

FIGS. 5 and 6 demonstrate the results of VEGF-induced migration assay of HDMECs, showing the inhibitory activity of TSP-1 variants of the present inventionas compared to that of known human TSP-1 and 173aa (3TSR domain) positive control. FIG. 5 shows the results of the migration inhibition assay using 2 nM and 20 nM of TSP-1 variants. FIG. 6 shows the results of the migration inhibition assay using 0.5 nM and 2 nM of TSP-1 variants.

FIG. 7 demonstrates variant protein alignment to the previously known proteins.

### DESCRIPTION OF THE PREFERRED EMBODIMENTS

The present invention is of novel thrombospondin-1 (TSP-1) variant polypeptides and polynucleotides encoding same, which can be used for the treatment of a wide range of diseases, in which TSP-1 activity and/or expression modulates disease onset and/or progression, such that treating the disease may involve influencing TSP-1 activity and/or expression. Examples of TSP-1-related diseases include, but are not limited to, cancer, such as, primary cancer and tumor cell metastasis. “TSP-1-related disease(s)” refers also to diseases in which anti-angiogenic activity plays a favorable role, including but not limited to, diseases having abnormal quality and/or quantity of vascularization as a characteristic feature, such as cancer for example, including but not limited to, breast cancer, colon cancer, pancreatic cancer, ovarian cancer, bladder cancer, lung cancer, melanoma, brain cancer, and other solid tumors and metastatic cancers. Other examples of TSP-1-related diseases include, but are not limited to, wound healing and inflammation, such as rheumatoid arthritis.

According to still other preferred embodiments, the present invention optionally and preferably encompasses any amino acid sequence or fragment thereof encoded by a nucleic acid sequence corresponding to a splice variant protein as described herein, including any oligopeptide or peptide relating to such an amino acid sequence or fragment, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

In another embodiment, the present invention relates to bridges, tails, heads and/or insertions, and/or analogs, homologs and derivatives of such peptides. Such bridges, tails, heads and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein a “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now,contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

As used herein the phrase “known protein” refers to a known database provided sequence of a specific protein, including, but not limited to, SwissProt (ca.expasy.org/), National Center of Biotechnology Information (NCBI) (www.ncbi.nim.nih.gov/), PIR (pir.georgetown.edu/), A Database of Human Unidentified Gene-Encoded Large Proteins [HUGE<www.kazusa.or.jp/huge>], Nuclear Protein Database [npd.hgu.mrc.ac.uk], human mitochondrial protein database (bioinfo.nist.gov:8080/examples/servlets/index.html), and University Protein Resource (Uni Prot) (www.expasy.uniprot.org/).

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

Optionally and preferably, a bridge between a tail or a head or a unique insertion, and a “known protein” portion of a variant, comprises at least about 10 amino acids, more preferably at least about 20 amino acids, most preferably at least about 30 amino acids, and even more preferably at least about 40 amino acids, in which at least one amino acid is from the tail/head/insertion and at least one amino acid is from the “known protein” portion of a variant. Also optionally, the bridge may comprise any number of amino acids from about 10 to about 40 amino acids (for example, 10, 11, 12, 13, 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_{—l P}1): a sequence starting from any of amino acid numbers 49-x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49-x (for example) is not less than 1, nor 50+((n−2)−x) (for example) greater than the total sequence length.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

According to still other preferred embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or compositions are described below.

Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

Thus, the present invention provides isolated polynucleotides each encoding a polypeptide which is at least 50%, at least 55%, at least 60%, at least 65%, at least 70%, at least 75%, at least 80%, %, at least 85%, %, at least 90%, at least 95% or more, say 100% identical to a polypeptide sequence listed in the Examples section or sequence listing, as determined using the LALIGN software of EMBnet Switzerland (http://www.ch.embnet.org/index.html) using default parameters.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the polynucleotides of the present invention.

Expression of the Polynucleotide Sequence of the Present Invention

To enable cellular expression of the polynucleotides of the present invention, a nucleic acid construct (or an “expression vector”) according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. NatI. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (±), pGL3, PzeoSV2 (±), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., includingRetro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa califomica nucleopolyhedrovirus (AcMNPV) as described in Liang CY et al., 2004 (Arch Virol. 149: 51-60).

Recombinant viral vectors are useful for in vivo expression of the polynucleotide sequence of the present invention since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of the present invention can also include sequences engineered to enhance stability, production, purification, yield or toxicity of the expressed peptide. For example, the expression of a fusion protein or a cleavable fusion protein comprising TSP-1 variant of the present invention and a heterologous protein can be engineered. Such a fusion protein can be designed so that the fusion protein can be readily isolated by affinity chromatography; e.g., by immobilization on a column specific for the heterologous protein. Where a cleavage site is engineered between the TSP-1 moiety and the heterologous protein, the TSP-1 moiety can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned hereinabove, a variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of the present invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. patent application Ser. No: 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hspl7.5-E or hspl7.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by the present invention.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Not withstanding the above, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Expression systems

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Variant Recombinant Expression Vectors and Host Cells

Another aspect of the invention pertains to vectors, preferably expression vectors, containing a nucleic acid encoding a variant protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., variant proteins, mutant forms of variant proteins, fusion proteins, etc.).

The recombinant expression vectors of the invention can be designed for production of variant proteins in prokaryotic or eukaryotic cells. For example, variant proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or carboxyl terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) and pTrcHis (Invitrogen Life Technologies) that fuse glutathione S-transferase (GST), maltose E binding protein, protein A or 6xHis, respectively, to the target recombinant protein.

Examples of suitable inducible non-fusion E. coli expression vectors include pTrc (Amrann et al., (1988) Gene 69:301-315).

One strategy to maximize recombinant protein expression in E. coli is to express the protein in host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another optional strategy to solve codon bias is by using BL21 -codon plus bacterial strains (Invitrogen) or Rosetta bacterial strain (Novagen), as these strains contain extra copies of rare E. coli tRNA genes.

In another embodiment, the expression vector encoding for the variant protein is a yeast expression vector. Examples of vectors for expression in yeast Saccharomyces cerivisae include pYepSecl (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, variant protein can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195), plRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory,Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for variant protein. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, variant protein can be produced in bacterial cells such as E. coli, insect cells, yeast or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding variant protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) variant protein. Accordingly, the invention further provides methods for producing variant protein using the host cells of the invention. In one embodiment, the method comprises culturing the host cell of the present invention (into which a recombinant expression vector encoding variant protein has been introduced) in a suitable medium such that variant protein is produced. In another embodiment, the method further comprises isolating variant protein from the medium or the host cell.

For efficient production of the protein, it is preferable to place the nucleotide sequences encoding the variant protein under the control of expression control sequences optimized for expression in a desired host. For example, the sequences may include optimized transcriptional and/or translational regulatory sequences (such as altered Kozak sequences).

Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH-) within the peptide may be substituted, for example, by N-methylated bonds (—N(CH3)-CO—), ester bonds (—C(R)H—C—O—O—C(R)—N—), ketomethylen bonds (—CO—CH2-), *-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (No1), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Since the peptides of the present invention are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

Peptide sequences which exhibit high therapeutic activity, such as by competing with wild type signaling proteins of the same signaling pathway, can be also uncovered using computational biology. Software programs useful for displaying three-dimensional structural models, such as RIBBONS (Carson, M., 1997. Methods in Enzymology 277, 25), O (Jones, TA. et al., 1991. Acta Crystallogr. A47, 110), DINO (DINO: Visualizing Structural Biology (2001) http://www.dino3d.org); and QUANTA, INSIGHT, SYBYL, MACROMODE, ICM, MOLMOL, RASMOL and GRASP (reviewed in Kraulis, J., 1991. Appl Crystallogr. 24, 946) can be utilized to model interactions between the polypeptides of the present invention and prospective peptide sequences to thereby identify peptides which display the highest probability of binding for example to a respective ligand (e.g., IL-10). Computational modeling of protein-peptide interactions has been successfully used in rational drug design, for further details, see Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109, and Mauro M J. et al., 2002. J Clin Oncol. 20, 325-34.

Antibodies

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Monoclonal antibody development may optionally be performed according to any method that is known in the art. The method described below is provided for the purposes of description only and is not meant to be limiting in any way.

Step 1: Immunization of Mice and Selection of Mouse Donors for Generation of Hybridoma Cells

Producing mAb requires immunizing an animal, usually a mouse, by injection of an antigen X to stimulate the production of antibodies targeted against X. Antigen X can be the whole protein or any sequence thereof that gives rise to a determinant. According to the present invention, optionally and preferably such antigens may include but are not limited to any variant described herein or a portion thereof, including but not limited to any head, tail, bridge or unique insertion, or a bridge to such head, tail or unique insertion, or any other epitope described herein according to the present invention. Injection of peptides requires peptide design (with respect to protein homology, antigenicity, hydrophilicity, and synthetic suitability) and synthesis. The antigen is optionally and preferably prepared for injection either by emulsifying the antigen with Freund's adjuvant or other adjuvants or by homogenizing a gel slice that contains the antigen. Intact cells, whole membranes, and microorganisms are sometimes optionally used as immunogens. Other immunogens or adjuvants may also optionally be used.

In general, mice are immunized every 2-3 weeks but the immunization protocols are heterogeneous. When a sufficient antibody titer is reached in serum, immunized mice are euthanized and the spleen removed to use as a source of cells for fusion with myeloma cells.

Step 2: Screening of Mice for Antibody Production

After several weeks of immunization, blood samples are optionally and preferably obtained from mice for measurement of serum antibodies. Several techniques have been developed for collection of small volumes of blood from mice (Loeb and Quimby 1999). Serum antibody titer is determined with various techniques, such as enzyme-linked immunosorbent assay (ELISA) and flow cytometry, and/or immunoassays for example (for example a Western blot may optionally be used). If the antibody titer is high, cell fusion can optionally be performed. If the titer is too low, mice can optionally be boosted until an adequate response is achieved, as determined by repeated blood sampling. When the antibody titer is high enough, mice are commonly boosted by injecting antigen without adjuvant intraperitoneally or intravenously (via the tail veins) 3 days before fusion but 2 weeks after the previous immunization. Then the mice are euthanized and their spleens removed for in vitro hybridoma cell production.

Step 3: Preparation of Myeloma Cells

Fusing antibody-producing spleen cells, which have a limited life span, with cells derived from an immortal tumor of lymphocytes (myeloma) results in a hybridoma that is capable of unlimited growth. Myeloma cells are immortalized cells that are optionally and preferably cultured with 8-azaguanine to ensure their sensitivity to the hypoxanthine-aminopterin-thymidine (HAT) selection medium used after cell fusion. The selection growth medium contains the inhibitor aminopterin, which blocks synthetic pathways by which nucleotides are made. Therefore, the cells must use a bypass pathway to synthesize nucleic acids, a pathway that is defective in the myeloma cell line to which the normal antibody-producing cells are fused. Because neither the myeloma nor the antibody-producing cell will grow on its own, only hybrid cells grow. The HAT medium allows only the fused cells to survive in culture. A week before cell fusion, myeloma cells are grown in 8-azaguanine. Cells must have high viability and rapid growth.

The antibody forming cells are isolated from the mouse's spleen and are then fused with a cancer cell (such as cells from a myeloma) to make them immortal, which means that they will grow and divide indefinitely. The resulting cell is called a hybridoma.

Step 4: Fusion of Myeloma Cells with Immune Spleen Cells and Antibody Screening

Single spleen cells from the immunized mouse are fused with the previously prepared myeloma cells. Fusion is accomplished by co-centrifuging freshly harvested spleen cells and myeloma cells in polyethylene glycol, a substance that causes cell membranes to fuse. Alternatively, the cells are centrifuged, the supernatant is discarded and PEG is then added. The cells are then distributed to 96 well plates containing feeder cells derived from saline peritoneal washes of mice. Feeder cells are believed to supply growth factors that promote growth of the hybridoma cells (Quinlan and Kennedy 1994). Commercial preparations that result from the collection of media supporting the growth of cultured cells and contain growth factors are available that can be used in lieu of mouse-derived feeder cells. It is also possible to use murine bone marrow-derived macrophages as feeder cells (Hoffman and others 1996).

Once hybridoma colonies reach a satisfactory cell count, the plates are assayed by an assay, eg ELISA or a regular immunoassay such as RIA for example, to determine which colonies are secreting antibodies to the immunogen. Cells from positive wells are isolated and expanded. Conditioned medium from each colony is retested to verify the stability of the hybridomas (that is, they continue to produce antibody).

Step 5: Cloning of Hybridoma Cell Lines by “Limiting Dilution” or Expansion and Stabilization of Clones by Ascites Production

At this step new, small clusters of hybridoma cells from the 96 well plates can be grown in tissue culture followed by selection for antigen binding or grown by the mouse ascites method with cloning at a later time.

For prolonged stability of the antibody-producing cell lines, it is necessary to clone and then recline the chosen cells. Cloning consists of subcloonng the cells by either limiting dilution at an average of less than one cell in each culture well or by platingout the cells in a thin layer of semisolid agar of methyl cellulose or by single-cell manipulation. At each stage, cultures are assayed for production of the appropriate antibodies.

Step 6: Antibody Purification

The secreted antibodies are optionally purified, preferably by one or more column chromatography steps and/or some other purification method, including but not limited to ion exchange, affinity, hydrophobic interaction, and gel permeation chromatography. The operation of the individual chromatography step, their number and their sequence is generally tailored to the specific antibody and the specific application.

Large-scale antibody production may also optionally and preferably be performed according to the present invention. Two non-limiting, illustrative exemplary methods are described below for the purposes of description only and are not meant to be limiting in any way.

In vivo production may optionally be performed with ascites fluid in mice. According to this method, hybridoma cell lines are injected into the peritoneal cavity of mice to produce ascitic fluid (ascites) in its abdomen; this fluid contains a high concentration of antibody.

An exemplary in vitro method involves the use of culture flasks. In this method, monoclonal antibodies can optionally be produced from the hybridoma using gas permeable bags or cell culture flasks.

Antibody Engineering in Phage Display Libraries

PCT Application No. WO 94/18219, and its many US equivalents, including U.S. Pat. No. 6,096,551, all of which are hereby incorporated by reference as if fully set forth herein, describes methods for producing antibody libraries using universal or randomized immunoglobulin light chains, by using phage display libraries. The method involves inducing mutagenesis in a complementarity determining region (CDR) of an immunoglobulin light chain gene for the purpose of producing light chain gene libraries for use in combination with heavy chain genes and gene libraries to produce antibody libraries of diverse and novel immunospecificities. The method comprises amplifying a CDR portion of an immunoglobulin light chain gene by polymerase chain reaction (PCR) using a PCR primer oligonucleotide. The resultant gene portions are inserted into phagemids for production of a phage display library,. wherein the engineered light chains are displayed by the phages, for example for testing their binding specificity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. A scFv antibody fragment is an engineered antibody derivative that includes heavy- and light chain variable regions joined by a peptide linker. The minimal size of antibody molecules are those that still comprise the complete antigen binding site. ScFv antibody fragments are potentially more effective than unmodified IgG antibodies. The reduced size of 27-30 kDa permits them to penetrate tissues and solid tumors more readily. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR) CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)]. Optionally, there may be 1, 2 or 3 CDRs of different chains, but preferably there are 3 CDRs of 1 chain. The chain could be the heavy or the light chain.

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intem. Rev. Immunol. 13, 65-93 (1995).

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

A “variant-treatable” disease refers to any disease that is treatable by using a splice variant of any of the therapeutic proteins according to the present invention. “Treatment” also encompasses prevention, amelioration, elimination and control of the disease and/or pathological condition. The diseases for which such variants may be useful therapeutic agents are described in greater detail below for each of the variants. The variants themselves are described by “cluster” or by gene, as these variants are splice variants of known proteins. Therefore, a “cluster-related disease” or a “protein-related disease” refers to a disease that may be treated by a particular protein, with regard to the description of such diseases below a therapeutic protein variant according to the present invention.

The term “biologically active”, as used herein, refers to a protein having structural, regulatory, or biochemical functions of a naturally occurring molecule. Likewise, “immunologically active” refers to the capability of the natural, recombinant, or synthetic ligand, or any oligopeptide thereof, to induce a specific immune response in appropriate animals or cells and to bind with specific antibodies.

The term “modulate”, as used herein, refers to a change in the activity of at least one receptor mediated activity. For example, modulation may cause an increase or a decrease in protein activity, binding characteristics, or any other biological, functional or immunological properties of a ligand.

Protein Modifications

Fusion Proteins

A fusion protein may be prepared from a variant protein according to the present invention by fusion with a portion of an immunoglobulin comprising a constant region of an immunoglobulin. More preferably, the portion of the immunoglobulin comprises a heavy chain constant region which is optionally and more preferably a human heavy chain constant region. The heavy chain constant region is most preferably an IgG heavy chain constant region, and optionally and most preferably is an Fc chain, most preferably an IgG Fc fragment that comprises CH2 and CH3 domains. Although any IgG subtype may optionally be used, the IgG1 subtype is preferred. The Fc chain may optionally be a known or “wild type” Fc chain, or alternatively may be mutated. Non-limiting, illustrative, exemplary types of mutations are described in U.S. patent application Ser. No. 20060034852, published on Feb. 16, 2006, hereby incorporated by reference as if fully set forth herein. The term “Fc chain” also optionally comprises any type of Fc fragment.

Several of the specific amino acid residues that are important for antibody constant region-mediated activity in the IgG subclass have been identified. Inclusion, substitution or exclusion of these specific amino acids therefore allows for inclusion or exclusion of specific immunoglobulin constant region-mediated activity. Furthermore, specific changes may result in aglycosylation for example and/or other desired changes to the Fc chain. At least some changes may optionally be made to block a function of Fc which is considered to be undesirable, such as an undesirable immune system effect, as described in greater detail below.

Non-limiting, illustrative examples of mutations to Fc which may be made to modulate the activity of the fusion protein include the following changes (given with regard to the Fc sequence nomenclature as given by Kabat, from Kabat EA et al: Sequences of Proteins of Immunological Interest. US Department of Health and Human Services, NIH, 1991): 220C ->S; 233-238 ELLGGP ->EAEGAP; 265D ->A, preferably in combination with 434N ->A; 297N ->A (for example to block N-glycosylation); 318-322 EYKCK ->AYACA; 330-331AP ->SS; or a combination thereof (see for example M. Clark, “Chemical Immunol and Antibody Engineering”, pp 1-31 for a description of these mutations and their effect). The construct for the Fc chain which features the above changes optionally and preferably comprises a combination of the hinge region with the CH2 and CH3 domains.

The above mutations may optionally be implemented to enhance desired properties or alternatively to block non-desired properties. For example, aglycosylation of antibodies was shown to maintain the desired binding functionality while blocking depletion of T-cells or triggering cytokine release, which may optionally be undesired functions (see M. Clark, “Chemical Immunol and Antibody Engineering”, pp 1-31). Substitution of 331 proline for serine may block the ability to activate complement, which may optionally be considered an undesired function (see M. Clark, “Chemical Immunol and Antibody Engineering”, pp 1-31). Changing 330 alanine to serine in combination with this change may also enhance the desired effect of blocking the ability to activate complement.

Residues 235 and 237 were shown to be involved in antibody-dependent cell-mediated cytotoxicity (ADCC), such that changing the block of residues from 233-238 as described may also block such activity if ADCC is considered to be an undesirable function.

Residue 220 is normally a cysteine for Fc from IgG1, which is the site at which the heavy chain forms a covalent linkage with the light chain. Optionally, this residue may be changed to a serine, to avoid any type of covalent linkage (see M. Clark, “Chemical Immunol and Antibody Engineering”, pp 1-31).

The above changes to residues 265 and 434 may optionally be implemented to reduce or block binding to the Fc receptor, which may optionally block undesired functionality of Fc related to its immune system functions (see “Binding site on Human IgG1 for Fc Receptors”, Shields et al, vol 276, pp 6591-6604, 2001).

The above changes are intended as illustrations only of optional changes and are not meant to be limiting in any way. Furthermore, the above explanation is provided for descriptive purposes only, without wishing to be bound by a single hypothesis.

Addition of Groups

If a variant according to the present invention is a linear molecule, it is possible to place various functional groups at various points on the linear molecule which are susceptible to or suitable for chemical modification. Functional groups can be added to the termini of linear forms of the variant. In some embodiments, the functional groups improve the activity of the variant with regard to one or more characteristics, including but not limited to, improvement in stability, penetration (through cellular membranes and/or tissue barriers), tissue localization, efficacy, decreased clearance, decreased toxicity, improved selectivity, improved resistance to expulsion by cellular pumps, and the like. For convenience sake and without wishing to be limiting, the free N-terminus of one of the sequences contained in the compositions of the invention will be termed as the N-terminus of the composition, and the free C-terminal of the sequence will be considered as the C-terminus of the composition. Either the C-terminus or the N-terminus of the sequences, or both, can be linked to a carboxylic acid functional groups or an amine functional group, respectively.

Non-limiting examples of suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the active ingredient attached thereto into a cell, for example, by reducing the hydrophilicity and increasing the lipophilicity of the active ingredient, these being an example for “a moiety for transport across cellular membranes”.

These moieties can optionally and preferably be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a composition of the present invention is protected, preferably with a methyl, ethyl, benzyl or substituted benzyl ester, more preferably as a benzyl ester.

Non-limiting, illustrative examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include but are not limited to acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—, Adamantan, naphtalen, myristoleyl, toluen, biphenyl, cinnamoyl, nitrobenzoy, toluoyl, furoyl, benzoyl, cyclohexane, norbomane, or Z-caproic. In order to facilitate the N-acylation, one to four glycine residues can be present in the N-terminus of the molecule.

The carboxyl group at the C-terminus of the compound can be protected, for example, by a group including but not limited to an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are optionally independently an aliphatic, substituted aliphatic, benzyl, substituted benzyl, aryl or a substituted aryl group. In addition, taken together with the nitrogen atom, R₂ and R₃ can optionally form a C4 to C8 heterocyclic ring with from about 0-2 additional heteroatoms such as nitrogen, oxygen or sulfur. Non-limiting suitable examples of suitable heterocyclic rings include piperidinyl, pyrrolidinyl, morpholino, thiomorpholino or piperazinyl. Examples of C-terminal protecting groups include but are not limited to —NH₂, —NHCH₃, —N(CH₃) ₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

Substitution by Peptidomimetic Moieties

A “peptidomimetic organic moiety” can optionally be substituted for amino acid residues in the composition of this invention both as conservative and as non-conservative substitutions. These moieties are also termed “non-natural amino acids” and may optionally replace amino acid residues, amino acids or act as spacer groups within the peptides in lieu of deleted amino acids. The peptidomimetic organic moieties optionally and preferably have steric, electronic or configurational properties similar to the replaced amino acid and such peptidomimetics are used to replace amino acids in the essential positions, and are considered conservative substitutions. However such similarities are not necessarily required. According to preferred embodiments of the present invention, one or more peptidomimetics are selected such that the composition at least substantially retains its physiological activity as compared to the native variant protein according to the present invention.

Peptidomimetics may optionally be used to inhibit degradation of the peptides by enzymatic or other degradative processes. The peptidomimetics can optionally and preferably be produced by organic synthetic techniques. Non-limiting examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988)); LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron. Lett. 29:5057-5060 (1988), Kemp et al., Tetrahedron Lett. 29:4935-4938 (1988) and Kemp et al., J. Org. Chem. 54:109-115 (1987). Other suitable but exemplary peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable exemplary peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl- phenylalanine and (2R, 3R)-methyl-phenylalanine (Kazmierski and Hruby, Tetrahedron Lett. (1991)).

Exemplary, illustrative but non-limiting non-natural amino acids include beta-amino acids (beta3 and beta2), homo-amino acids, cyclic amino acids, aromatic amino acids, Pro and Pyr derivatives, 3-substituted Alanine derivatives, Glycine derivatives, ring-substituted Phe and Tyr Derivatives, linear core amino acids or diamino acids. They are available from a variety of suppliers, such as Sigma-Aldrich (USA) for example.

Chemical Modifications

In the present invention any part of a variant protein may optionally be chemically modified, i.e. changed by addition of functional groups. For example the side amino acid residues appearing in the native sequence may optionally be modified, although as described below alternatively other part(s) of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible.

The amino acid of any of the sequence regions of the molecule can optionally be modified according to any one of the following exemplary types of modification (in the peptide conceptually viewed as “chemically modified”). Non-limiting exemplary types of modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a protein or peptide according to the present invention, refers to a protein or peptide where at least one of its amino acid residues is modified either by natural processes, such as processing or other post-translational modifications, or by chemical modification techniques which are well known in the art. Examples of the numerous known modifications typically include, but are not limited to: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications optionally include the addition of a cycloalkane moiety to a biological molecule, such as a protein, as described in PCT Application No. WO 2006/050262, hereby incorporated by reference as if fully set forth herein. These moieties are designed for use with biomolecules and may optionally be used to impart various properties to proteins.

Furthermore, optionally any point on a protein may be modified. For example, pegylation of a glycosylation moiety on a protein may optionally be performed, as described in PCT Application No. WO 2006/050247, hereby incorporated by reference as if fully set forth herein. One or more polyethylene glycol (PEG) groups may optionally be added to 0-linked and/or N-linked glycosylation. The PEG group may optionally be branched or linear. Optionally any type of water-soluble polymer may be attached to a glycosylation site on a protein through a glycosyl linker.

Altered Glycosylation

Variant proteins of the invention may be modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or 0-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to variant proteins of the invention is conveniently accomplished by altering the amino acid sequence of the protein such that it contains one or more of the above-described tripeptide sequences (for N-linked glycosylation sites). The alteration may also be made by the addition of, or substitution by, one or more serine or threonine residues in the sequence of the original protein (for O-linked glycosylation sites). The protein's amino acid sequence may also be altered by introducing changes at the DNA level.

Another means of increasing the number of carbohydrate moieties on proteins is by chemical or enzymatic coupling of glycosides to the amino acid residues of the protein. Depending on the coupling mode used, the sugars may be attached to (a) arginine and histidine, (b) free carboxyl groups, (c) free sulthydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, CRC Crit. Rev. Biochem., 22: 259-306 (1981).

Removal of any carbohydrate moieties present on variant proteins of the invention may be accomplished chemically or enzymatically. Chemical deglycosylation requires exposure of the protein to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on proteins can be achieved by the use of a variety of endo- and exo-glycosidases as described by Thotakura et al., Meth. Enzymol., 138: 350 (1987).

Methods Of Treatment

As mentioned hereinabove the novel therapeutic protein variants of the present invention and compositions derived therefrom (i.e., peptides, oligonucleotides) can be used to treat cluster or protein-related diseases, disorders or conditions.

Thus, according to an additional aspect of the present invention there is provided a method of treating cluster or protein-related disease, disorder or condition in a subject.

As used herein the term “treating” refers to preventing, curing, reversing, attenuating, alleviating, minimizing, suppressing or halting the deleterious effects of the above-described diseases, disorders or conditions.

Treating, according to the present invention, can be effected by specifically upregulating or alternatively downregulating the expression of at least one of the polypeptides of the present invention in the subject.

Optionally, upregulation may be effected by administering to the subject at least one of the polypeptides of the present invention (e.g., recombinant or synthetic) or an active portion thereof, as described herein. However, since the bioavailability of large polypeptides may potentially be relatively small due to high degradation rate and low penetration rate, administration of polypeptides is preferably confined to small peptide fragments (e.g.,. about 100 amino acids). The polypeptide or peptide may optionally be administered in as part of a pharmaceutical composition, described in more detail below.

It will be appreciated that treatment of the above-described diseases according to the present invention may be combined with other treatment methods known in the art (i.e., combination therapy). Thus, treatment of malignancies using the agents of the present invention may be combined with, for example, radiation therapy, antibody therapy and/or chemotherapy.

Alternatively or additionally, an upregulating method may optionally be effected by specifically upregulating the amount (optionally expression) in the subject of at least one of the polypeptides of the present invention or active portions thereof.

As is mentioned hereinabove and in the Examples section which follows, the biomolecular sequences of this aspect of the present invention may be used as valuable therapeutic tools in the treatment of diseases, disorders or conditions in which altered activity or expression of the wild-type (known) gene product is known to contribute to disease, disorder or condition onset or progression.

It will be appreciated that the polypeptides of the present invention may also have agonistic properties. These include increasing the stability of the Wild Type full length TSP-1, protection from proteolysis and modification of the pharmacokinetic properties of TSP-1 (i.e., increasing its half-life, while decreasing the clearance thereof. As such, the biomolecular sequences of this aspect of the present invention may be used to treat conditions or diseases in which the wild-type gene product plays a favorable role, for example, increasing angiogenesis in cases of diabetes or ischemia.

Upregulating expression of the therapeutic protein or polypeptide variants of the present invention may be effected via the administration of at least one of the exogenous polynucleotide sequences of the present invention, ligated into a nucleic acid expression construct (as described in greater detail hereinabove) designed for expression of coding sequences in eukaryotic cells (e.g., mammalian cells), as described above. Accordingly, the exogenous polynucleotide sequence may be a DNA or RNA sequence encoding the variants of the present invention or active portions thereof.

It will be appreciated that the nucleic acid construct can be administered to the individual employing any suitable mode of administration including in vivo gene therapy (e.g., using viral transformation as described hereinabove). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

Such cells (i.e., which are transfected with the nucleic acid construct of the present invention) can be any suitable cells, such as kidney, bone marrow, keratinocyte, lymphocyte, adult stem cells, cord blood cells, embryonic stem cells which are derived from the individual and are transfected ex vivo with an expression vector containing the polynucleotide designed to express the polypeptide of the present inevntion as described hereinabove.

Administration of the ex vivo transfected cells of the present invention can be effected using any suitable route such as intravenous, intra peritoneal, intra kidney, intra gastrointestinal track, subcutaneous, transcutaneous, intramuscular, intracutaneous, intrathecal, epidural and rectal. According to presently preferred embodiments, the ex vivo transfected cells of the present invention are introduced to the individual using intravenous, intra kidney, intra gastrointestinal track and/or intra peritoneal administrations.

The ex vivo transfected cells of the present invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu MZ, et al., Cell encapsulation with alginate and alpha-phenoxycinnamylidene-acetylated poly(allylamine). Biotechnol Bioeng. 2000, 70: 479-83, Chang TM and Prakash S. Procedures for microencapsulation of enzymes, cells and genetically engineered microorganisms. Mol Biotechnol. 2001, 17: 249-60, and Lu MZ, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

It will be appreciated that the present methodology may also be effected by specifically upregulating the expression of the variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-5R gene, which include (i.e., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Design and synthesis of oligonucleotides which can be used according to the present invention are described hereinbelow and by Sazani and Kole (2003) Progress in Moleclular and Subcellular Biology 31:217-239.

Pharmaceutical Compositions And Delivery Thereof

The present invention features a pharmaceutical composition comprising a therapeutically effective amount of a therapeutic agent according to the present invention, which is preferably a therapeutic protein variant as described herein. Optionally and alternatively, the therapeutic agent could be an antibody or an oligonucleotide that specifically recognizes and binds to the therapeutic protein variant, but not to the corresponding full length known protein.

Alternatively, the pharmaceutical composition of the present invention includes a therapeutically effective amount of at least an active portion of a therapeutic protein variant polypeptide.

The pharmaceutical composition according to the present invention is preferably used for the treatment of cluster or protein-related disease, disorder or condition.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder. “Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the agent according to the present invention. This includes chronic and acute disorders or diseases including those pathological conditions which predispose the mammal to the disorder in question. Non-limiting examples of disorders to be treated herein are described with regard to specific examples given herein.

The term “therapeutically effective amount” refers to an amount of agent according to the present invention that is effective to treat a disease or disorder in a mammal. In the case of cancer, the therapeutically effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the agent may prevent growth and/or kill existing cancer cells, it may be cytostatic and/or cytotoxic. For cancer therapy, efficacy can, for example, be measured by assessing the time to disease progression (TTP) and/or determining the response rate (RR).

The therapeutic agents of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or eniulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated. Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc. Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Immunogenic Compositions

A therapeutic agent according to the present invention may optionally be a molecule, which promotes a specific immunogenic response against at least one of the polypeptides of the present invention in the subject. The molecule can be polypeptide variants of the present invention, a fragment derived therefrom or a nucleic acid sequence encoding thereof. Although such a molecule can be provided to the subject per se, the agent is preferably administered with an immunostimulant in an immunogenic composiiton. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes into which the compound is incorporated (see e.g., U.S. Pat. No. 4,235,877). Vaccine preparation is generally described in, for example, M. F. Powell and M. J. Newman, eds., “Vaccine Design (the subunit and adjuvant approach),” Plenum Press (NY, 1995).

Illustrative immunogenic compositions may contain DNA encoding one or more of the polypeptides as described above, such that the polypeptide is generated in situ. The DNA may be present within any of a variety of delivery systems known to those of ordinary skill in the art, including nucleic acid expression systems (see below), bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the subject (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Nati. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

It will be appreciated that an immunogenic composition may comprise both a polynucleotide and a polypeptide component. Such immunogenic compositions may provide for an enhanced immune response.

Any of a variety of immunostimulants may be employed in the immunogenic compositions of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2,-7, or -12, may also be used as adjuvants.

The adjuvant composition may be designed to induce an immune response predominantly of the Th1 type. High levels of Th1-type cytokines (e.g., IFN-.gamma., TNF.alpha., IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of an immunogenic composition as provided herein, the subject will support an immune response that includes Th1- and Th2-type responses. The levels of these cytokines may be readily assessed using standard assays. For a review of the families of cytokines, see Mosmann and Coffinan, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly ThI response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720.

A delivery vehicle may be employed within the immunogenic composition of the present invention to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Dendritic cells are highly potent APCs (Banchereau and Steinman, Nature 392:245-251, 1998) and have been shown to be effective as a physiological adjuvant for eliciting prophylactic or therapeutic antitumor immunity (see Timmernan and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within an immunogenic composition (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF.alpha. to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNF.alpha., CD40 ligand, LPS, flt3 ligand and/or other compound(s) that induce differentiation, maturation and proliferation of dendritic cells.

Dendritic cells are categorized as “immature” and “mature” cells, which allows a simple way to discriminate between two well characterized phenotypes. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with at least one polynucleotide encoding a polypeptide of the present invention, such that variant II, or an immunogenic portion thereof, is expressed on the cell surface. Such transfection may take place ex vivo, and a composition comprising such transfected cells may then be used for therapeutic purposes, as described herein. Alternatively, a gene delivery vehicle that targets a dendritic or other antigen presenting cell may be administered to the subject, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with a polypeptide of the present inventio, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule) such as described above. Alternatively, a dendritic cell may be pulsed with a non-conjugated immunological partner, separately or in the presence of the polypeptide.

Preferred embodiments of the present invention encompass novel naturally occurring secreted (i.e., extracellular) and non-secreted (i.e., intracellular or membranal) variants of genes and gene products, which, as is described in the Examples section which follows, play pivotal roles in disease onset and progression. As such these variants can be used for a wide range of therapeutic uses.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

Description of the methodology undertaken to uncover the biomolecular sequences of the present invention and uses therefor

Human ESTs and cDNAs were obtained from GenBank versions 136 (Jun. 15, 2003 ncbi “dot” nih “dot” gov/genbank/release “dot” notes/gb136 “dot” release “dot” notes) and NCBI genome assembly of April 2003. Novel splice variants were predicted using the LEADS clustering and assembly system as described in U.S. Pat. No: 6,625,545, U.S. patent application Ser. No. 10/426,002, both of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

Brief description of the methodology used to obtain annotative sequence information is summarized infra (for detailed description see U.S. patent application Ser. No. 10/426,002, published as US20040101876 on May 27 2004).

The ontological annotation approach—An ontology refers to the body of knowledge in a specific knowledge domain or discipline such as molecular biology, microbiology, immunology, virology, plant sciences, pharmaceutical chemistry, medicine; neurology, endocrinology, genetics, ecology, genomics, proteomics, cheminformatics, pharmacogenomics, bioinformatics, computer sciences, statistics, mathematics, chemistry, physics and artificial intelligence.

An ontology includes domain-specific concepts—referred to, herein, as sub-ontologies. A sub-ontology may be classified into smaller and narrower categories. The ontological annotation approach is effected as follows.

First, biomolecular (i.e., polynucleotide or polypeptide) sequences are computationally clustered according to a progressive homology range, thereby generating a plurality of clusters each being of a predetermined homology of the homology range.

Progressive homology is used to identify meaningful homologies among biomolecular sequences and to thereby assign new ontological annotations to sequences, which share requisite levels of homologies. Essentially, a biomolecular sequence is assigned to a specific cluster if displays a predetermined homology to at least one member of the cluster (i.e., single linkage). A “progressive homology range” refers to a range of homology thresholds, which progress via predetermined increments from a low homology level (e.g. 35%) to a high homology level (e.g. 99%).

Following generation of clusters, one or more ontologies are assigned to each cluster. Ontologies are derived from an annotation preassociated with at least one biomolecular sequence of each cluster; and/or generated by analyzing (e.g., text-mining) at least one biomolecular sequence of each cluster thereby annotating biomolecular sequences.

The hierarchical annotation approach—“Hierarchical annotation” refers to any ontology and subontology, which can be hierarchically ordered, such as, a tissue expression hierarchy, a developmental expression hierarchy, a pathological expression hierarchy, a cellular expression hierarchy, an intracellular expression hierarchy, a taxonomical hierarchy, a functional hierarchy and so forth.

The hierarchical annotation approach is effected as follows. First, a dendrogram representing the hierarchy of interest is computationally constructed. A “dendrogram” refers to a branching diagram containing multiple nodes and representing a hierarchy of categories based on degree of similarity or number of shared characteristics.

Each of the multiple nodes of the dendrogram is annotated by at least one keyword describing the node, and enabling literature and database text mining, such as by using publicly available text mining software. A list of keywords can be obtained from the GO Consortium (www.geneontlogy.org). However, measures are taken to include as many keywords, and to include keywords which might be out of date. For example, for tissue annotation, a hierarchy is built using all available tissue/libraries sources available in the GenBank, while considering the following parameters: ignoring GenBank synonyms, building anatomical hierarchies, enabling flexible distinction between tissue types (normal versus pathology) and tissue classification levels (organs, systems, cell types, etc.).

In a second step, each of the biomolecular sequences is assigned to at least one specific node of the dendrogram.

The biomolecular sequences can be annotated biomolecular sequences, unannotated biomolecular sequences or partially annotated biomolecular sequences.

Annotated biomolecular sequences can be retrieved from pre-existing annotated databases as described hereinabove.

For example, in GenBank, relevant annotational information is provided in the definition and keyword fields. In this case, classification of the annotated biomolecular sequences to the dendrogram nodes is directly effected. A search for suitable annotated biomolecular sequences is performed using a set of keywords which are designed to classify the biomolecular sequences to the hierarchy (i.e., same keywords that populate the dendrogram).

In cases where the biomolecular sequences are unannotated or partially annotated, extraction of additional annotational information is effected prior to classification to dendrogram nodes. This can be effected by sequence alignment, as described hereinabove. Alternatively, annotational information can be predicted from structural studies. Where needed, nucleic acid sequences can be transformed to amino acid sequences to thereby enable more accurate annotational prediction.

Finally, each of the assigned biomolecular sequences is recursively classified to nodes hierarchically higher than the specific nodes, such that the root node of the dendrogram encompasses the full biomolecular sequence set, which can be classified according to a certain hierarchy, while the offspring of any node represent a partitioning of the parent set.

For example, a biomolecular sequence found to be specifically expressed in “rhabdomyosarcoma”, will be classified also to a higher hierarchy level, which is “sarcoma”, and then to “Mesenchymal cell tumors” and finally to a highest hierarchy level “Tumor”. In another example, a sequence found to be differentially expressed in endometrium cells, will be classified also to a higher hierarchy level, which is “uterus”, and then to “women genital system” and to “genital system” and finally to a highest hierarchy level “genitourinary system”. The retrieval can be performed according to each one of the requested levels.

Annotating gene expression according to relative abundance—Spatial and temporal gene annotations are also assigned by comparing relative abundance in libraries of different origins. This approach can be used to find genes, which are differentially expressed in tissues, pathologies and different developmental stages. In principal, the presentation of a contig in at least two tissues of interest is determined and significant over or under representation of the contig in one of the at least two tissues is assessed to identify differential expression. Significant over or under representation is analyzed by statistical pairing.

Annotating spatial and temporal expression can also be effected on splice variants. This is effected as follows. First, a contig which includes exonal sequence presentation of the at least two splice variants of the gene of interest is obtained. This contig is assembled from a plurality of expressed sequences. Then, at least one contig sequence region, unique to a portion (i.e., at least one and not all) of the at least two splice variants of the gene of interest, is identified. Identification of such unique sequence region is effected using computer alignment software. Finally, the number of the plurality of expressed sequences in the tissue having the at least one contig sequence region is compared with the number of the plurality of expressed sequences not-having the at least one contig sequence region, to thereby compare the expression level of the at least two splice variants of the gene of interest in the tissue.

Data concerning therapies, indications and possible pharmacological activities of the polypeptides of the present invention was obtained from PharmaProject (PJB Publications Ltd 2003 www “dot” pjbpubs “dot” com/cms “dot” asp?pageid=340) and public databases, including LocusLink (www “dot” genelynx “dot” org/cgi-bin/resource?res=locuslink) and Swissprot (www “dot” ebi “dot” ac “dot” uk/swissprot/index “dot” html). Functional structural analysis of the polypeptides of the present invention was effected using Interpro domain analysis software (Interpro default parameters, the analyses that were run are HMMPfam, HMMSmart, ProfileScan, FprintScan, and BlastProdom). Subecilular localization was analysed using ProLoc software (Einat Hazkani-Covo, Erez Y. Levanon, Galit Rotman, Dan Graur, Amit Novik. Evolution of multicellularity in metazoa: comparative analysis of the subcellular localization of proteins in Saccharomyces, Drosophila and Caenorhabditis. Cell Biology International (2004;28(3):171-8).

Identifying gene products by interspecies sequence comparison—The present inventors have designed and configured a method of predicting gene expression products based on interspecies sequence comparison. Specifically, the method is based on the identification of conserved alternatively spliced exons for which there might be no supportive expression data.

Alternatively spliced exons have unique characteristics differentiating them from constitutively spliced ones. Using machine-learning techniques a combination of such characteristics was elucidated that defines alternatively spliced exons with very high probability. Any human exon having this combination of characteristics is therefore predicted to be alternatively spliced. Using this method, the present inventors were able to detect putative splice variants that are not supported by human ESTs.

The method is effected as follows. First, alternatively spliced exons of a gene of interest are identified by scoring exon sequences of the gene of interest according to at least one sequence parameter as follows: (i) exon length—conserved alternatively spliced exons are relatively shorter than constitutively spliced ones; (ii) division by 3 —alternatively spliced exons are cassette exons that are sometimes inserted and sometimes skipped; Since alternatively spliced exons frequently contain sequences that regulate their splicing important parameters for scoring alternatively spliced exons include (iii) conservation level to a non-human ortholohgous sequence; (iv) length of conserved intron sequences upstream of each of the exon sequences; (v) length of conserved intron sequences downstream of each of the exon sequences; (vi) conservation level of the intron sequences upstream of each of the exon sequences; and (vii) conservation level of the intron sequences downstream of each of the exon sequences.

Exon sequences scoring above a predetermined threshold represent alternatively spliced exons of the gene of interest.

Once alternatively spliced exons are identified, the chromosomal location of each of the alternatively spliced exons is analyzed with respect to coding sequence of the gene of interest to thereby predict expression products of the gene of interest. When performed along with computerized means, mass prediction of gene products can be effected.

In addition, for identifying new gene products by interspecies sequence comparison, the expressed sequences derived from non-human species can be used for new human splice variants prediction.

### Example 2

### Description for Cluster Humthrom

Cluster HUMTHROM features 5 transcripts the names for which are given in Table 1. The selected protein variants are given in Table 2.

TABLE 1
Transcripts of interest

Transcript Name

HUMTHROM_1_T12 (SEQ ID NO:1)
HUMTHROM_1_T14 (SEQ ID NO:2)
HUMTHROM_1_T15 (SEQ ID NO:3)
HUMTHROM_1_T17 (SEQ ID NO:4)
HUMTHROM_1_T32 (SEQ ID NO:5)

TABLE 2
Proteins of interest

| | Corresponding |
|---|---|
| Protein Name | Transcript(s) |
| HUMTHROM_1_P8 | HUMTHROM_1_T12 |
| (SEQ ID NO:48) | (SEQ ID NO:1) |
| HUMTHROM_1_P10 | HUMTHROM_1_T15 |
| (SEQ ID NO:49) | (SEQ ID NO:3) |
| HUMTHROM_1_P12 | HUMTHROM_1_T17 |
| (SEQ ID NO:50) | (SEQ ID NO:4) |
| HUMTHROM_1_P22 | HUMTHROM_1_T32 |
| (SEQ ID NO:51) | (SEQ ID NO:5) |
| HUMTHROM_1_P27 | HUMTHROM_1_T14 |
| (SEQ ID NO:52) | (SEQ ID NO:2) |

These sequences are variants of the known protein Thrombospondin 1 precursor (SEQ ID NO:44) (SwissProt accession identifier TSP-1_HUMAN), referred to herein as the previously known protein.

Protein Thrombospondin 1 precursor (SEQ ID NO:44) is known or believed to have the following function(s): Adhesive glycoprotein that mediates cell-to-cell and cell-to-matrix interactions. Can bind to fibrinogen, fibronectin, laminin, type V collagen and integrins alpha-V/beta-1, alpha- V/beta-3 and alpha-Ilb/beta-3. Known polymorphisms for this sequence are as shown in Table 3.

TABLE 3
Amino acid mutations for Known Protein

| SNP position(s) on | |
|---|---|
| amino acid sequence | Comment |
| 84 | T -> A |
| 523 | T -> A |

The previously known protein also has the following indication(s) and/or potential therapeutic use(s): Cancer, general. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Angiogenesis inhibitor; Thrombospondin antagonist. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anticancer, other; Imaging agent; Recombinant, other.

The following GO Annotation(s) apply to the previously known protein. The following annotation(s) were found: development, which are annotation(s) related to Biological Process; endopeptidase inhibitor activity; signal transducer activity, which are annotation(s) related to Molecular Function; and extracellular region, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

Acording to the present invention, TSP variants can be used for treatment of primary and metastatic cancer, targeting a broad spectrum of cancers, including but not limited to prostate cancer, renal cancer, cervical carcinomas, breast cancer, colon and colorectal cancer, pancreatic cancer, ovarian cancer, bladder cancer, lung cancer, melanoma, brain cancer, soft tissue sarcomas, lymphomas, head-and-neck, glioblastomas, and other tumors and metastatic cancers. This includes the use of the TSP-1 variants in this invention as monotherapy for cancer, or in combination therapy with any of various other cytotoxic agents, or anti-angiogenic and/or anti-tumor agents.

TSP variants of the present invention can be used for treatment of retinal angiogenesis in a number of human ocular diseases, such as diabetic retinopathy, retinopathy of prematurity, and age-related macular degeneration.

As noted above, cluster HUMTHROM features 5 transcript(s), which were listed in Table 1 above. These transcript(s) encode for protein(s) which are variant(s) of protein Thrombospondin 1 precursor (SEQ ID NO:44). A description of each variant protein according to the present invention is now provided.

Variant protein HUMTHROM_{—}1_P8 (SEQ ID NO:48) according to the present it is encoded by transcript(s) HUMTHROM_{—}1_T12 (SEQ ID NO:1).

The localization of the variant protein was determined according to results from a number of different software programs and analyses, including analyses from SignalP and other specialized programs. The variant protein is believed to be secreted.

Variant protein HUMTHROM_{—}1_P8 (SEQ ID NO:48) also has the following non-silent SNPs (Single Nucleotide Polymorphisms) as listed in Table 4 (given according to their position(s) on the amino acid sequence, with the alternative amino acid)(s) listed;).

TABLE 4
Amino acid mutations

| SNP position(s) on amino | Alternative |
|---|---|
| acid sequence | amino acid(s) |
| 42 | K -> |
| 79 | V -> M |
| 163 | D -> G |
| 181 | V -> |
| 237 | S -> N |
| 329 | E -> G |
| 478 | A -> |

The glycosylation sites of variant protein HUMTHROM_{—}1_P8 (SEQ ID NO:48), as compared to the known protein Thrombospondin 1 precursor (SEQ ID NO:44), are described in Table 5 (given according to their position(s) on the amino acid sequence in the first column; the second column indicates whether the glycosylation site is present in the variant protein; and the last column indicates whether the position is different on the variant protein).

TABLE 5
Glycosylation site(s)

| Position(s) on known | Present in | Position(s) on variant |
|---|---|---|
| amino acid sequence | variant protein? | protein |
| 248 | Yes | 248 |
| 360 | Yes | 360 |
| 385 | Yes | 385 |
| 394 | Yes | 394 |
| 438 | Yes | 438 |
| 441 | Yes | 441 |
| 450 | Yes | 450 |
| 498 | No | |
| 507 | No | |
| 708 | No | |
| 1067 | No | |

The variant protein has the following domains, as determined by using InterPro. The domains are described in Table 6.

TABLE 6
InterPro domain(s)

| | Analysis | |
|---|---|---|
| Domain description | type | Position(s) on protein |
| Thrombospondin, subtype 1 | FPrintScan | 436-449, 454-465, 473-484 |
| Thrombospondin, type I | HMMPfam | 383-428, 439-489 |
| von Willebrand factor, type C | HMMPfam | 318-372 |
| Thrombospondin, type I | HMMSmart | 382-429, 438-490 |
| Thrombospondin, N-terminal | HMMSmart | 24-221 |
| von Willebrand factor, type C | HMMSmart | 318-372 |
| Thrombospondin, type I | ProfileScan | 379-429, 435-490 |
| von Willebrand factor, type C | ProfileScan | 316-373 |
| von Willebrand factor, type C | ScanRegExp | 336-372 |

Variant protein HUMTHROM_{—}1_P8 (SEQ ID NO:48) is encoded by the transcript HUMTHROM_{—}1_T12 (SEQ ID NO:1). The coding portion of transcript HUMTHROM_{—}1_T12 (SEQ ID NO:1) starts at position 326 and ends at position 2059. The transcript also has the following SNPs as listed in Table 7 (given according to their position on the nucleotide sequence, with the alternative nucleic acid listed).

TABLE 7
Nucleic acid SNPs

| SNP position(s) on | Alternative |
|---|---|
| nucleotide sequence | nucleic acid(s) |
| 21 | G -> C |
| 151 | G -> A |
| 188 | T -> C |
| 451 | G -> |
| 560 | G -> A |
| 813 | A -> G |
| 868 | C -> |
| 1035 | G -> A |
| 1311 | A -> G |
| 1615 | G -> A |
| 1735 | C -> T |
| 1757 | G -> |
| 2199 | A -> G |
| 2204 | C -> T |
| 2431 | T -> C |
| 2519 | C -> |
| 2528 | G -> |
| 2599 | A -> G |
| 2675 | C -> |
| 2727 | C -> |
| 2731 | A -> G |
| 3230 | C -> G |
| 3230 | C -> |
| 3500 | T -> C |
| 3505 | G -> |
| 3536 | A -> |
| 3550 | A -> |
| 3603 | A -> G |
| 3661 | G -> C |
| 3892 | C -> |
| 3932 | A -> G |
| 3982 | A -> |
| 4105 | T -> C |
| 4183 | A -> |
| 4224 | C -> T |
| 4423 | G -> A |
| 4450 | T -> A |
| 4490 | A -> T |
| 4559 | C -> A |
| 4643 | A -> T |
| 4730 | G -> A |
| 4808 | C -> G |
| 4821 | C -> T |
| 4856 | A -> C |
| 5033 | T -> G |
| 5121 | T -> |
| 5135 | T -> |
| 5251 | T -> G |
| 5251 | T -> |
| 5275 | T -> |
| 5420 | C -> A |
| 5420 | C -> |
| 5489 | T -> C |
| 5489 | T -> |
| 5605 | T -> C |
| 5606 | C -> T |
| 5674 | T -> |
| 5777 | A -> G |
| 5852 | T -> |
| 5974 | C -> G |
| 6052 | T -> G |
| 6057 | T -> G |
| 6125 | A -> G |

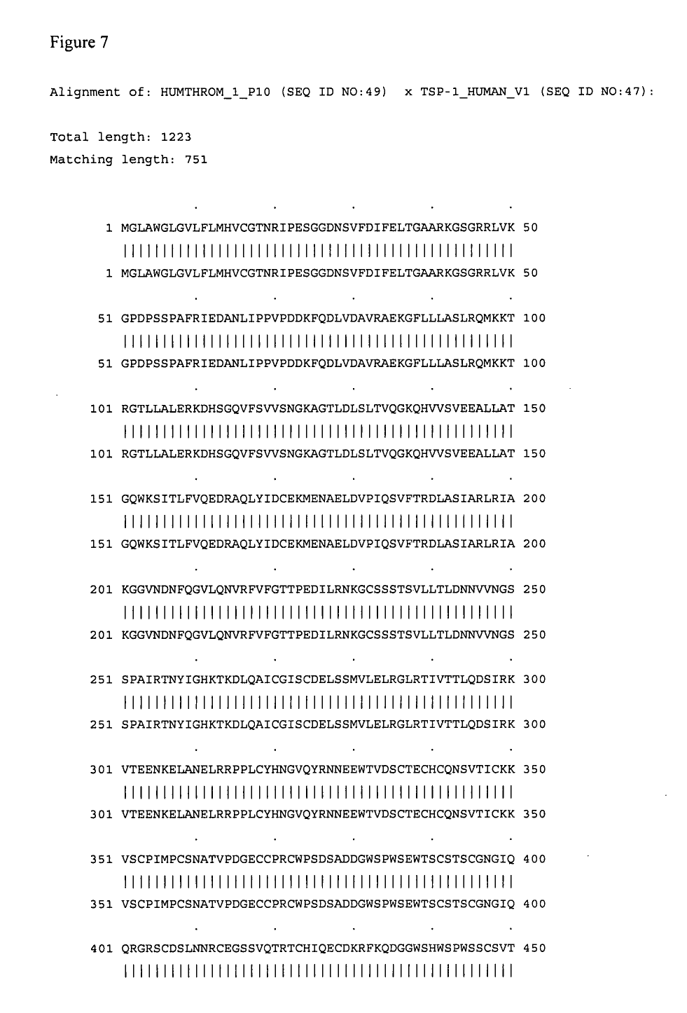

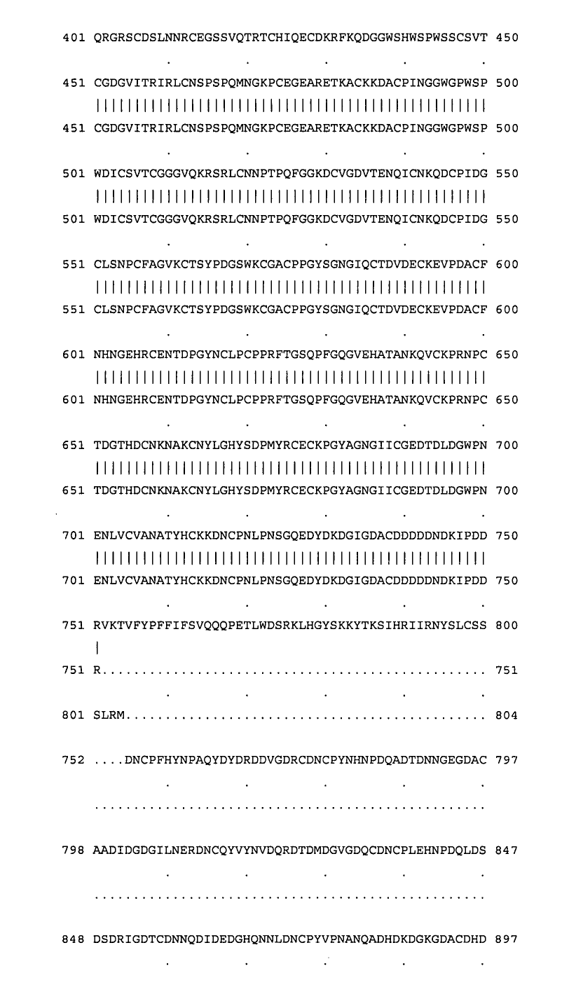

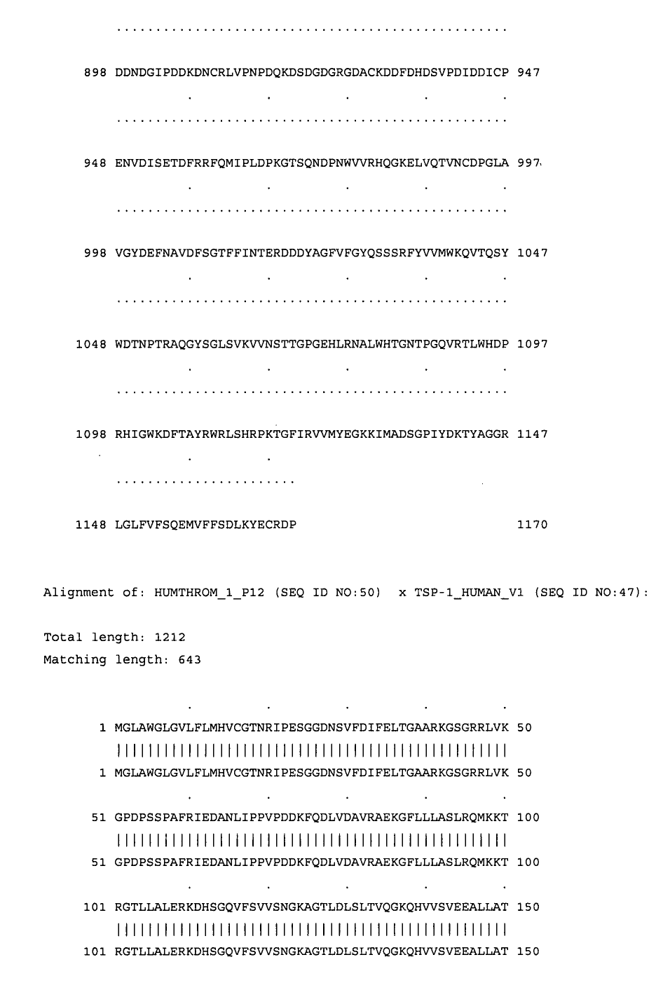

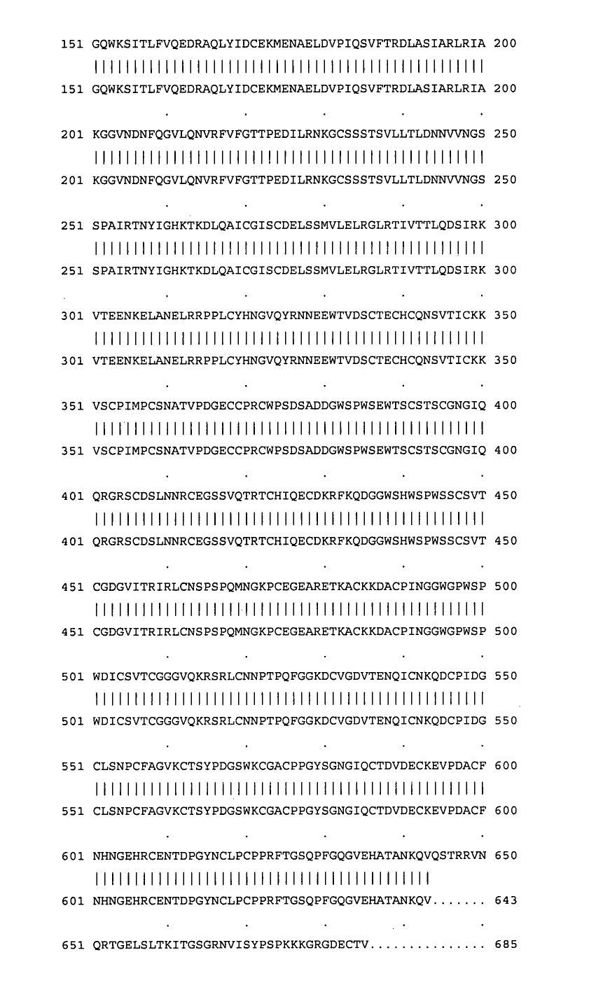

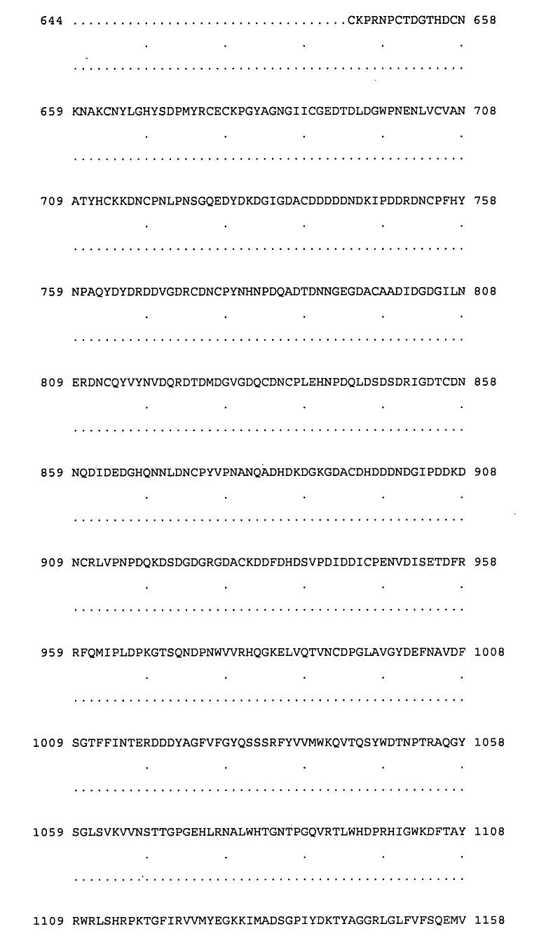

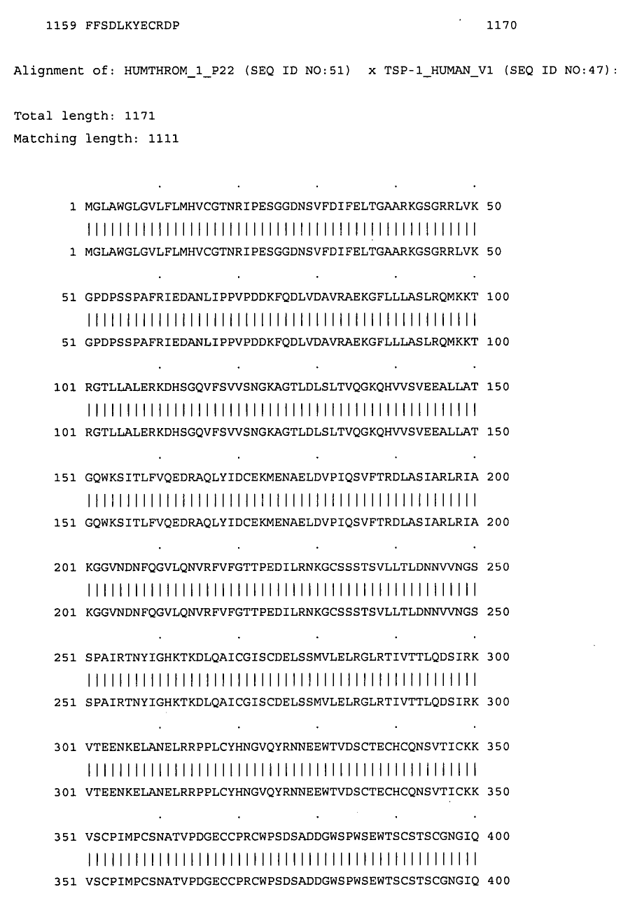

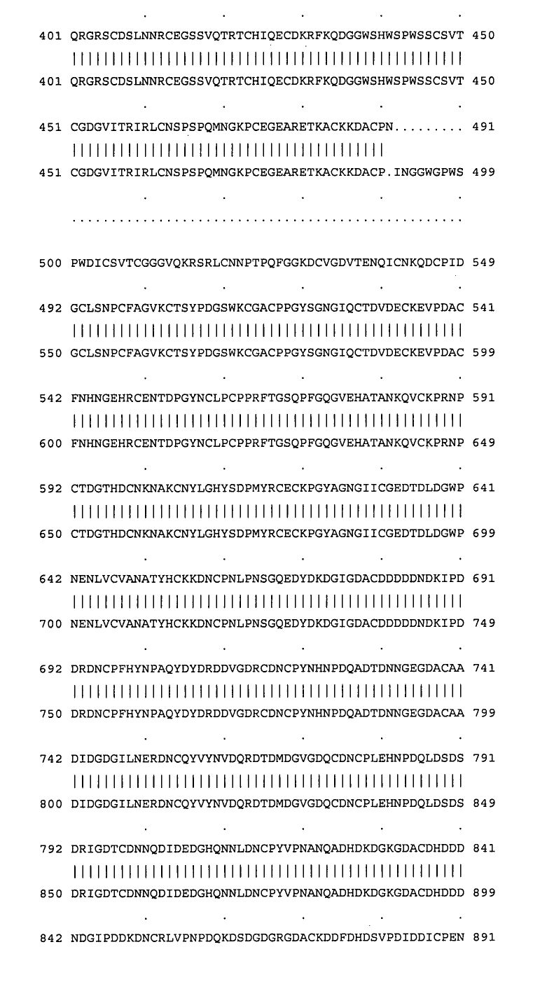

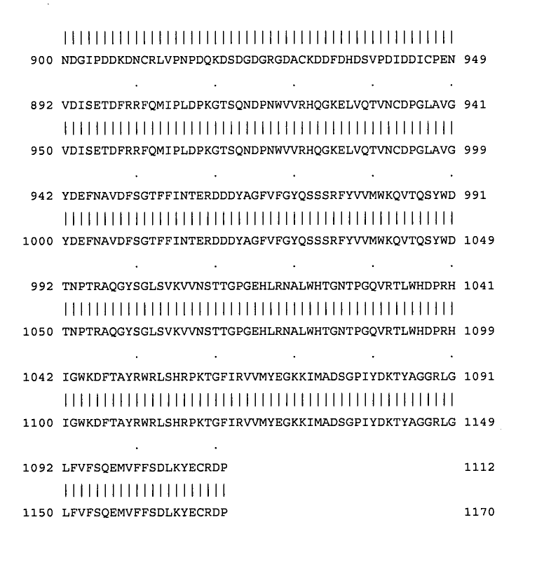

Variant protein HUMTHROM_{—}1_P10 (SEQ ID NO:49) according to the present invention is encoded by transcript HUMTHROM_{—}1_T15 (SEQ ID NO:3). One or more alignments to one or more previously published protein sequences are shown in FIG. 7. A brief description of the relationship of the variant protein according to the present invention to each such aligned protein is as follows:

1. Comparison report between HUMTHROM_{—}1_P10 (SEQ ID NO:49) and TSP-1_HUMAN_V1 (SEQ ID NO:47):

A. An isolated chimeric polypeptide encoding for HUMTHROM_{—}1_P10 (SEQ ID NO:49), comprising a first amino acid sequence being at least 90% homologous to MGLAWGLGVLFLMHVCGTNRIPESGGDNSVFDIFELTGAARKGSGRRLVKG PDPSSPAFRIEDANLIPPVPDDKFQDLVDAVRAEKGFLLLASLRQMKKTRGTL LALERKDHSGQVFSVVSNGKAGTLDLSLTVQGKQHVVSVEEALLATGQWKS ITLFVQEDRAQLYIDCEKMENAELDVPIQSVFTRDLASIARLRIAKGGVNDNF QGVLQNVRFVFGTTPEDILRNKGCSSSTSVLLTLDNNVVNGSSPAIRTNYIGH KTKDLQAICGISCDELSSMVLELRGLRTIVTTLQDSfRKVTEENKELANELRRP PLCYHNGVQYRNNEEWTVDSCTECHCQNSVTICKKVSCPIMPCSNATVPDGE CCPRCWPSDSADDGWSPWSEWTSCSTSCGNGIQQRGRSCDSLNNRCEGSSVQ TRTCHIQECDKRFKQDGGWSHWSPWSSCSVTCGDGVITRIRLCNSPSPQMNG KPCEGEARETKACKKDACPINGGWGPWSPWDICSVTCGGGVQKRSRLCNNP TPQFGGKDCVGDVTENQICNKQDCPIDGCLSNPCFAGVKCTSYPDGSWKCGA CPPGYSGNGIIQCTDVDECKEVPDACFNHNGEHRCENTDPGYNCLPCPPRFTG SQPFGQGVEHATANKQVCKPRNPCTDGTHDCNKNAKCNYLGHYSDPMYRC ECKPGYAGNGIICGEDTDLDGWPNENLVCVANATYHCKKDNCPNLPNSGQE DYDKDGIGDACDDDDDNDKIPDDR corresponding to amino acids 1-751 of TSP-1_HUMAN_V1 (SEQ ID NO:47), which also corresponds to amino acids 1-751 of HUMTHROM_{—}1_P10 (SEQ ID NO:49), and a second amino acid sequence being at least 70%, optionally at least 80%, preferably at least 85%, more preferably at least 90% and most preferably at least 95% homologous to a polypeptide having the sequence VKTVFYPFFIFSVQQQPETLWDSRKLHGYSKKYTKSIHRIIRNYSLCSSSLRM corresponding to amino acids 752-804 of HUMTHROM_{—}1_P10 (SEQ ID NO:49), wherein said first amino acid sequence and second amino acid sequence are contiguous and in a sequential order.

B. An isolated polypeptide encoding for an edge portion of HUMTHROM_{—}1_P10 (SEQ ID NO:49), comprising an amino acid sequence being at least 70%, optionally at least about 80%, preferably at least about 85%, more preferably at least about 90% and most preferably at least about 95% homologous to the sequence VKTVFYPFFIFSVQQQPETLWDSRKLHGYSKKYTKSIHRIIRNYSLCSSSLRM of HUMTHROM_{—}1_P10 (SEQ ID NO:49).

It should be noted that the known protein sequence (TSP-1_HUMAN) has one or more changes than the sequence given at the end of the application and named as being the amino acid sequence for TSP-1_HUMAN_V1 (SEQ ID NO:47). These changes were previously known to occur and are listed in the table below.

TABLE 8
Changes to TSP-1_HUMAN_V1 (SEQ ID NO: 47)

| SNP position on | |
|---|---|
| amino acid sequence | Type of change |
| 84 | conflict |

The localization of the variant protein was determined according to results from a number of different software programs and analyses, including analyses from SignalP and other specialized programs. The variant protein is believed to be secreted.

Variant protein HUMTHROM_{—}1_P10 (SEQ ID NO:49) also has the following non-silent SNPs (Single Nucleotide Polymorphisms) as listed in Table 9, (given according to their position(s) on the amino acid sequence, with the alternative amino acid(s) listed).

TABLE 9
Amino acid mutations

| SNP position(s) on amino | Alternative |
|---|---|
| acid sequence | amino acid(s) |
| 42 | K -> |
| 79 | V -> M |
| 163 | D -> G |
| 181 | V -> |
| 237 | S -> N |
| 329 | E -> G |
| 478 | A -> |
| 523 | T -> A |
| 600 | F -> S |
| 629 | P -> |
| 632 | Q -> |
| 656 | D -> G |
| 681 | G -> |
| 699 | P -> |
| 700 | N -> S |

The glycosylation sites of variant protein HUMTHROM_{—}1_P10 (SEQ ID NO:49), as compared to the known protein Thrombospondin 1 precursor (SEQ ID NO:44), are described in Table 10 (given according to their position(s) on the amino acid sequence in the first column; the second column indicates whether the glycosylation site is present in the variant protein; and the last column indicates whether the position is different on the variant protein).

TABLE 10
Glycosylation site(s)

| Position(s) on known | Present in | Position(s) on variant |
|---|---|---|
| amino acid sequence | variant protein? | protein |
| 248 | Yes | 248 |
| 360 | Yes | 360 |
| 385 | Yes | 385 |
| 394 | Yes | 394 |
| 438 | Yes | 438 |
| 441 | Yes | 441 |
| 450 | Yes | 450 |
| 498 | Yes | 498 |
| 507 | Yes | 507 |
| 708 | Yes | 708 |
| 1067 | No | |

The variant protein has the following domains, as determined by using InterPro. The domains are described in Table 11.

TABLE 11
InterPro domain(s)

| | Analysis | |
|---|---|---|
| Domain description | type | Position(s) on protein |
| Thrombospondin, subtype 1 | FPrintScan | 436-449, 454-465, 473-484 |
| EGF-like | HMMPfam | 650-689 |
| Thrombospondin, type I | HMMPfam | 383-428, 439-489, 496-546 |
| von Willebrand factor, type C | HMMPfam | 318-372 |
| Thrombospondin type 3 repeat | HMMPfam | 691-706, 727-739 |
| EGF-like calcium-binding | HMMSmart | 542-587, 588-645 |
| Type I EGF | HMMSmart | 550-587, 591-645, 649-690 |
| Thrombospondin, type I | HMMSmart | 382-429, 438-490, 495-547 |
| Thrombospondin, N-terminal | HMMSmart | 24-221 |
| von Willebrand factor, type C | HMMSmart | 318-372 |
| Thrombospondin, type I | ProfileScan | 379-429, 435-490, 492-547 |
| von Willebrand factor, type C | ProfileScan | 316-373 |
| EGF-like | ScanRegExp | 676-689 |
| von Willebrand factor, type C | ScanRegExp | 336-372 |

Variant protein HUMTHROM_{—}1_P10 (SEQ ID NO:49) is encoded by the transcript HUMTHROM_{—}1_T15 (SEQ ID NO:3). The coding portion of transcript HUMTHROM_{—}1_T15 (SEQ ID NO:3) starts at position 326 and ends at position 2737. The transcript also has the following SNPs as listed in Table 12 (given according to their position on the nucleotide sequence, with the alternative nucleic acid listed).

TABLE 12
Nucleic acid SNPs

| SNP position(s) on | Alternative |
|---|---|
| nucleotide sequence | nucleic acid(s) |
| 21 | G -> C |
| 151 | G -> A |
| 188 | T -> C |
| 451 | G -> |
| 560 | G -> A |
| 813 | A -> G |
| 868 | C -> |
| 1035 | G -> A |
| 1311 | A -> G |
| 1615 | G -> A |
| 1735 | C -> T |
| 1757 | G -> |
| 1892 | A -> G |
| 1897 | C -> T |
| 2124 | T -> C |
| 2212 | C -> |
| 2221 | G -> |
| 2292 | A -> G |
| 2368 | C -> |
| 2420 | C -> |
| 2424 | A -> G |
| 3490 | C -> G |
| 3490 | C -> |
| 3760 | T -> C |
| 3765 | G -> |
| 3796 | A -> |
| 3810 | A -> |
| 3863 | A -> G |
| 3921 | G -> C |
| 4152 | C -> |
| 4192 | A -> G |
| 4242 | A -> |
| 4365 | T -> C |
| 4443 | A -> |
| 4484 | C -> T |
| 4683 | G -> A |
| 4710 | T -> A |
| 4750 | A -> T |
| 4819 | C -> A |
| 4903 | A -> T |
| 4990 | G -> A |
| 5068 | C -> G |
| 5081 | C -> T |
| 5116 | A -> C |
| 5293 | T -> G |
| 5381 | T -> |
| 5395 | T -> |
| 5511 | T -> G |
| 5511 | T -> |
| 5535 | T -> |
| 5680 | C -> A |
| 5680 | C -> |
| 5749 | T -> C |
| 5749 | T -> |
| 5865 | T -> C |
| 5866 | C -> T |
| 5934 | T -> |
| 6037 | A -> G |
| 6112 | T -> |
| 6234 | C -> G |
| 6312 | T -> G |
| 6317 | T -> G |
| 6385 | A -> G |

Variant protein HUMTHROM_{—}1_P12 (SEQ ID NO:50) according to the present invention is encoded by transcript HUMTHROM_{—}1_T17 (SEQ ID NO:4). One or more alignments to one or more previously published protein sequences are shown in FIG. 7. A brief description of the relationship of the variant protein according to the present invention to each such aligned protein is as follows:

1. Comparison report between HUMTHROM_{—}1_P12 (SEQ ID NO:50) and TSP-1_HUMAN_V1 (SEQ ID NO:47):

A. An isolated chimeric polypeptide encoding for HUMTHROM_{—}1_P12 (SEQ ID NO:50), comprising a first amino acid sequence being at least 90% homologous to MGLAWGLGVLFLMHVCGTNRIPESGGDNSVFDIFELTGAARKGSGRRLVKG PDPSSPAFRIEDANLIPPVPDDKFQDLVDAVRAEKGFLLLASLRQMKKTRGTL LALERKDHSGQVFSVVSNGKAGTLDLSLTVQGKQHVVSVEEALLATGQWKS ITLFVQEDRAQLYIDCEKMENAELDVPIQSVFTRDLASIARLRIAKGGVNDNF QGVLQNVRFVFGTTPEDILRNKGCSSSTSVLLTLDNNVVNGSSPAIRTNYIGH KTKDLQAICGISCDELSSMVLELRGLRTIVTTLQDSIRKVTEENKELANELRRP PLCYHNGVQYRNNEEWTVDSCTECHCQNSVTICKKVSCPIMPCSNATVPDGE CCPRCWPSDSADDGWSPWSEWTSCSTSCGNGIQQRGRSCDSLNNRCEGSSVQ TRTCHIQECDKRFKQDGGWSHWSPWSSCSVTCGDGVITRIRLCNSPSPQMNG KPCEGEARETKACKKDACPINGGWGPWSPWDICSVTCGGGVQKRSRLCNNP TPQFGGKDCVGDVTENQICNKQDCPIDGCLSNPCFAGVKCTSYPDGS WKCGA CPPGYSGNGIQCTDVDECKEVPDACFNHNGEHRCENTDPGYNCLPCPPRFTG SQPFGQGVEHATANKQV corresponding to amino acids 1-643 of TSP-1_HUMAN_V1 (SEQ ID NO:47), which also corresponds to amino acids 1-643 of HUMTHROM_{—}1_P12 (SEQ ID NO:50), and a second amino acid sequence being at least 70%, optionally at least 80%, preferably at least 85%, more preferably at least 90% and most preferably at least 95% homologous to a polypeptide having the sequence QSTRRVNQRTGELSLTKITGSGRNVISYPSPKKKGRGDECTV corresponding to amino acids 644-685 of HUMTHROM_{—}1_P12 (SEQ ID NO:50), wherein said first amino acid sequence and second amino acid sequence are contiguous and in a sequential order.

B. An isolated polypeptide encoding for an edge portion of HUMTHROM_{—}1_P12 (SEQ ID NO:50), comprising an amino acid sequence being at least 70%, optionally at least about 80%, preferably at least about 85%, more preferably at least about 90% and most preferably at least about 95% homologous to the sequence QSTRRVNQRTGELSLTKITGSGRNVISYPSPKKKGRGDECTV of HUMTHROM_{—}1_P12 (SEQ ID NO:50).

The localization of the variant protein was determined according to results from a number of different software programs and analyses, including analyses from SignalP and other specialized programs. The variant protein is believed to be secreted.

Variant protein HUMTHROM_{—}1_P12 (SEQ ID NO:50) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein HUMTHROM_{—}1_P12 (SEQ ID NO:50) sequence des support for the deduced sequence of this variant protein according to the present invention).

TABLE 14
Amino acid mutations

| SNP position(s) on amino | |
|---|---|
| acid sequence | Alternative amino acid(s) |
| 42 | K -> |
| 79 | V -> M |
| 163 | D -> G |
| 181 | V -> |
| 237 | S -> N |
| 329 | E -> G |
| 478 | A -> |
| 523 | T -> A |
| 600 | F -> S |
| 629 | P -> |
| 632 | Q -> |

The glycosylation sites of variant protein HUMTHROM_{—}1_P12 (SEQ ID NO:50), as compared to the known protein Thrombospondin 1 precursor (SEQ ID NO:44), are described in Table 15 (given according to their position(s) on the amino acid sequence in the first column; the second column indicates whether the glycosylation site is present in the variant protein; and the last column indicates whether the position is different on the variant protein).

TABLE 15
Glycosylation site(s)

| Position(s) on known | Present in variant | Position(s) on variant |
|---|---|---|
| amino acid sequence | protein? | protein |
| 248 | Yes | 248 |
| 360 | Yes | 360 |
| 385 | Yes | 385 |
| 394 | Yes | 394 |
| 438 | Yes | 438 |
| 441 | Yes | 441 |
| 450 | Yes | 450 |
| 498 | Yes | 498 |
| 507 | Yes | 507 |
| 708 | No | |
| 1067 | No | |

The variant protein has the following domains, as determined by using InterPro. The domains are described in Table 16.

TABLE 16
InterPro domain(s)

| | Analysis | |
|---|---|---|
| Domain description | type | Position(s) on protein |
| Thrombospondin, subtype 1 | FPrintScan | 436-449, 454-465, 473-484 |
| Thrombospondin, type I | HMMPfam | 383-428, 439-489, 496-546 |
| von Willebrand factor, type C | HMMPfam | 318-372 |
| EGF-like calcium-binding | HMMSmart | 542-587, 588-632 |
| Type I EGF | HMMSmart | 550-587, 591-631 |
| Thrombospondin, type I | HMMSmart | 382-429, 438-490, 495-547 |
| Thrombospondin, N-terminal | HMMSmart | 24-221 |
| von Willebrand factor, type C | HMMSmart | 318-372 |
| Thrombospondin, type I | ProfileScan | 379-429, 435-490, 492-547 |
| von Willebrand factor, type C | ProfileScan | 316-373 |
| von Willebrand factor, type C | ScanRegExp | 336-372 |

Variant protein HUMTHROM_{—}1_P12 (SEQ ID NO:50) is encoded by the transcript HUMTHROM_{—}1_T17 (SEQ ID NO:4). The coding portion of transcript HUMTHROM_{—}1_T17 (SEQ ID NO:4 portion starts at position 326 and ends at position 2380. The transcript also has the following SNPs as listed in Table 17 (given according to their position on the nucleotide sequence, with the alternative nucleic acid listed).

TABLE 17
Nucleic acid SNPs

| SNP position(s) on | Alternative nucleic |
|---|---|
| nucleotide sequence | acid(s) |
| 21 | G -> C |
| 151 | G -> A |
| 188 | T -> C |
| 451 | G -> |
| 560 | G -> A |
| 813 | A -> G |
| 868 | C -> |
| 1035 | G -> A |
| 1311 | A -> G |
| 1615 | G -> A |
| 1735 | C -> T |
| 1757 | G -> |
| 1892 | A -> G |
| 1897 | C -> T |
| 2124 | T -> C |
| 2212 | C -> |
| 2221 | G -> |
| 2742 | A -> G |
| 2818 | C -> |
| 2870 | C -> |
| 2874 | A -> G |
| 3373 | C -> G |
| 3373 | C -> |
| 3643 | T -> C |
| 3648 | G -> |
| 3679 | A -> |
| 3693 | A -> |
| 3746 | A -> G |
| 3804 | G -> C |
| 4035 | C -> |
| 4075 | A -> G |
| 4125 | A -> |
| 4248 | T -> C |
| 4326 | A -> |
| 4367 | C -> T |
| 4566 | G -> A |
| 4593 | T -> A |
| 4633 | A -> T |
| 4702 | C -> A |
| 4786 | A -> T |
| 4873 | G -> A |
| 4951 | C -> G |
| 4964 | C -> T |
| 4999 | A -> C |
| 5176 | T -> G |
| 5264 | T -> |
| 5278 | T -> |
| 5394 | T -> G |
| 5394 | T -> |
| 5418 | T -> |
| 5563 | C -> A |
| 5563 | C -> |
| 5632 | T -> C |
| 5632 | T -> |
| 5748 | T -> C |
| 5749 | C -> T |
| 5817 | T -> |
| 5920 | A -> G |
| 5995 | T -> |
| 6117 | C -> G |
| 6195 | T -> G |
| 6200 | T -> G |
| 6268 | A -> G |

Variant protein HUMTHROM_{—}1_P22 (SEQ ID NO:51) according to the present is encoded by transcript HUMTHROM_{—}1_T32 (SEQ ID NO:5). One or more alignments to one or more previously published protein sequences are shown in FIG. 7. A brief description of the relationship of the variant protein according to the present invention to each such aligned protein is as follows:

1. Comparison report between HUMTHROM_{—}1_P22 (SEQ ID NO:51) and TSP-1_HUMAN_V1 (SEQ ID NO:47):

A. An isolated chimeric polypeptide encoding for HUMTHROM_{—}1_P22 (SEQ ID NO:51), comprising a first amino acid sequence being at least 90% homologous to WGLGVLFLMHVCGTNRIPESGGDNSVFDIFELTGAARKGSGRRLVKG SPAFRIEDANLIPPVPDDKFQDLVDAVRAEKGFLLLASLRQMKKTRGTL RKDHSGQVFSVVSNGKAGTLDLSLTVQGKQHVVSVEEALLATGQWKS VQEDRAQLYIDCEKMENAELDVPIQSVFTRDLASIARLRIAKGGVNDNF QNVRFVFGTTPEDILRNKGCSSSTSVLLTLDNNVVNGSSPAIRTNYIGH LQAICGISCDELSSMVLELRGLRTIVTTLQDSIRKVTEENKELANELRRP HNGVQYRNNEEWTVDSCTECHCQNSVTICKKVSCPIMPCSNATVPDGE CWPSDSADDGWSPWSEWTSCSTSCGNGIQQRGRSCDSLNNRCEGSSVQ HIQECDKRFKQDGGWSHWSPWSSCSVTCGDGVITRIRLCNSPSPQMNG KPCEGEARETKACKKDACP corresponding to amino acids 1-490 of TSP-1_HUMAN_V1 (SEQ ID NO:47), which also corresponds to amino acids 1-490 of HUMTHROM_{—}1_P22 (SEQ ID NO:51), a second bridging amino acid sequence comprising of N, and a third amino acid sequence being at least 90% homologous to GCLSNPCFAGVKCTSYPDGSWKCGACPPGYSGNGIQCTDVDECKEVPDACFN HNGEHRCENTDPGYNCLPCPPRFTGSQPFGQGVEHATANKQVCKPRNPCTDG THDCNKNAKCNYLGHYSDPMYRCECKPGYAGNGIICGEDTDLDGWPNENLV CVANATYHCKKDNCPNLPNSGQEDYDKDGIGDACDDDDDNDKIPDDRDNCP FHYNPAQYDYDRDDVGDRCDNCPYNHNPDQADTDNNGEGDACAADIDGDG ILNERDNCQYVYNVDQRDTDMDGVGDQCDNCPLEHNPDQLDSDSDRIGDTC DNNQDIDEDGHQNNLDNCPYVPNANQADHDKDGKGDACDHDDDNDGIPDD KDNCRLVPNPDQKDSDGDGRGDACKDDFDHDSVPDIDDICPENVDISETDFR RFQMIPLDPKGTSQNDPNWVVRHQGKELVQTVNCDPGLAVGYDEFNAVDFS GTFFINTERDDDYAGFVFGYQSSSRFYVVMWKQVTQSYWDTNPTRAQGYSG LSVKVVNSTTGPGEHLRNALWHTGNTPGQVRTLWHDPRHIGWKDFTAYRW RLSHRPKTGFIRVVMYEGKKIMADSGPIYDKTYAGGRLGLFVFSQEMVFFSD LKYECRDP corresponding to amino acids 550-1170 of TSP-1_HUMAN_V1 (SEQ ID NO:47), which also corresponds to amino acids 492-1112 of HUMTHROM_{—}1_P22 (SEQ ID NO:51), wherein said first amino acid sequence, second amino acid sequence and third amino acid sequence are contiguous and in a sequential order.

B. An isolated polypeptide encoding for an edge portion of HUMTHROM_{—}1_P22 (SEQ ID NO:51), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least three amino acids comprise PNG having a structure as follows (numbering according to HUMTHROM_{—}1_P22 (SEQ ID NO:51)): a sequence starting from any of amino acid numbers 490-x to 490; and ending at any of amino acid numbers 492+((n−2)−x), in which x varies from 0 to n−2.

The localization of the variant protein was determined according to results from a number of different software programs and analyses, including analyses from SignalP and other specialized programs. The variant protein is believed to be secreted.

Variant protein HUMTHROM_{—}1_P22 (SEQ ID NO:51) also has the following non-silent SNPs (Single Nucleotide Polymorphisms) as listed in Table 19, (given according to their position(s) on the amino acid sequence, with the alternative amino acid(s) listed).

TABLE 19
Amino acid mutations

| SNP position(s) on amino | |
|---|---|
| acid sequence | Alternative amino acid(s) |
| 42 | K -> |
| 79 | V -> M |
| 163 | D -> G |
| 181 | V -> |
| 237 | S -> N |
| 329 | E -> G |
| 478 | A -> |
| 542 | F -> S |
| 571 | P -> |
| 574 | Q -> |
| 598 | D -> G |
| 623 | G -> |
| 641 | P -> |
| 642 | N -> S |
| 808 | G -> |
| 900 | R -> |
| 910 | K -> |
| 915 | N -> |
| 933 | N -> D |
| 952 | G -> A |
| 1029 | P -> |
| 1042 | I -> M |
| 1059 | K -> |
| 1100 | V -> A |

The glycosylation sites of variant protein HUMTHROM_{—}1_P22 (SEQ ID NO:51), as compared to the known protein Thrombospondin 1 precursor (SEQ ID NO:44), are described in Table 20 (given according to their position(s) on the amino acid sequence in the first column; the second column indicates whether the glycosylation site is present in the variant protein; and the last column indicates whether the position is different on the variant protein).

TABLE 20
Glycosylation site(s)

| Position(s) on known | Present in variant | Position(s) on variant |
|---|---|---|
| amino acid sequence | protein? | protein |
| 248 | Yes | 248 |
| 360 | Yes | 360 |
| 385 | Yes | 385 |
| 394 | Yes | 394 |
| 438 | Yes | 438 |
| 441 | Yes | 441 |
| 450 | Yes | 450 |
| 498 | No | |
| 507 | No | |
| 650 | Yes | 650 |
| 1009 | Yes | 1009 |

The variant protein has the following domains, as determined by using InterPro. The domains are described in Table 21.

TABLE 21
InterPro domain(s)

| | Analysis | |
|---|---|---|
| Domain description | type | Position(s) on protein |
| Thrombospondin, subtype 1 | FPrintScan | 436-449, 454-465, 473-484 |
| EGF-like | HMMPfam | 592-631 |
| Thrombospondin, type I | HMMPfam | 383-428, 439-489 |
| von Willebrand factor, type C | HMMPfam | 318-372 |
| Thrombospondin type 3 repeat | HMMPfam | 633-648, 669-681, 682-697, |
| | | 705-717, 728-740, 741-756, |
| | | 764-776, 787-799, 802-817, |
| | | 825-837, 838-853, 861-873, |
| | | 874-889 |
| Thrombospondin, C-terminal | HMMPfam | 914-1112 |
| EGF-like calcium-binding | HMMSmart | 485-529, 530-587 |
| Type I EGF | HMMSmart | 492-529, 533-587, 591-632 |
| Thrombospondin, type I | HMMSmart | 382-429, 438-490 |
| Thrombospondin, N-terminal | HMMSmart | 24-221 |
| von Willebrand factor, type C | HMMSmart | 318-372 |
| Thrombospondin, type I | ProfileScan | 379-429, 435-490 |
| von Willebrand factor, type C | ProfileScan | 316-373 |
| EGF-like | ScanRegExp | 618-631 |
| von Willebrand factor, type C | ScanRegExp | 336-372 |

Variant protein HUMTHROM_{—}1_P22 (SEQ ID NO:51) is encoded by the transcript HUMTHROM_{—}1_T32 (SEQ ID NO:5). The coding portion of transcript HUMTHROM_{—}1_T32 (SEQ ID NO:5) portion starts at position 326 and ends at position 3661. The transcript also has the following SNPs as listed in Table 22 (given according to their position on the nucleotide sequence, with the alternative nucleic acid listed).

TABLE 22
Nucleic acid SNPs

| SNP position(s) on | Alternative nucleic |
|---|---|
| nucleotide sequence | acid(s) |
| 21 | G -> C |
| 151 | G -> A |
| 188 | T -> C |
| 451 | G -> |
| 560 | G -> A |
| 813 | A -> G |
| 868 | C -> |
| 1035 | G -> A |
| 1311 | A -> G |
| 1615 | G -> A |
| 1735 | C -> T |
| 1757 | G -> |
| 1950 | T -> C |
| 2038 | C -> |
| 2047 | G -> |
| 2118 | A -> G |
| 2194 | C -> |
| 2246 | C -> |
| 2250 | A -> G |
| 2749 | C -> G |
| 2749 | C -> |
| 3019 | T -> C |
| 3024 | G -> |
| 3055 | A -> |
| 3069 | A -> |
| 3122 | A -> G |
| 3180 | G -> C |
| 3411 | C -> |
| 3451 | A -> G |
| 3501 | A -> |
| 3624 | T -> C |
| 3702 | A -> |
| 3743 | C -> T |
| 3942 | G -> A |
| 3969 | T -> A |
| 4009 | A -> T |
| 4078 | C -> A |
| 4162 | A -> T |
| 4249 | G -> A |
| 4327 | C -> G |
| 4340 | C -> T |
| 4375 | A -> C |
| 4552 | T -> G |
| 4640 | T -> |
| 4654 | T -> |
| 4770 | T -> G |
| 4770 | T -> |
| 4794 | T -> |
| 4939 | C -> A |
| 4939 | C -> |
| 5008 | T -> C |
| 5008 | T -> |
| 5124 | T -> C |
| 5125 | C -> T |
| 5193 | T -> |
| 5296 | A -> G |
| 5371 | T -> |
| 5493 | C -> G |
| 5571 | T -> G |
| 5576 | T -> G |
| 5644 | A -> G |

Variant protein HUMTHROM_{—}1_P27 (SEQ ID NO:52) according to the present invention is encoded by transcript HUMTHROM_{—}1_T14 (SEQ ID NO:2).

The localization of the variant protein was determined according to results from a number of different software programs and analyses, including analyses from SignalP and other specialized programs. The variant protein is believed to be secreted.

Variant protein HUMTHROM_{—}1_P27 (SEQ ID NO:52) also has the following non-silent SNPs (Single Nucleotide Polymorphisms) as listed in Table 23, (given according to their position(s) on the amino acid sequence, with the alternative amino acid(s) listed).

TABLE 23
Amino acid mutations

| SNP position(s) on amino | |
|---|---|
| acid sequence | Alternative amino acid(s) |
| 42 | K -> |
| 79 | V -> M |
| 163 | D -> G |
| 181 | V -> |
| 237 | S -> N |
| 329 | E -> G |
| 478 | A -> |
| 523 | T -> A |

The glycosylation sites of variant protein HUMTHROM_{—}1_P27 (SEQ ID NO:52), as compared to the known protein Thrombospondin 1 precursor (SEQ ID NO:44), are described in Table 24 (given according to their position(s) on the amino acid sequence in the first column; the second column indicates whether the glycosylation site is present in the variant protein; and the last column indicates whether the position is different on the variant protein).

TABLE 24
Glycosylation site(s)

| Position(s) on known | Present in variant | Position(s) on variant |
|---|---|---|
| amino acid sequence | protein? | protein |
| 248 | Yes | 248 |
| 360 | Yes | 360 |
| 385 | Yes | 385 |
| 394 | Yes | 394 |
| 438 | Yes | 438 |
| 441 | Yes | 441 |
| 450 | Yes | 450 |
| 498 | Yes | 498 |
| 507 | Yes | 507 |
| 708 | No | |
| 1067 | No | |

The variant protein has the following domains, as determined by using InterPro. The domains are described in Table 25.

TABLE 25
InterPro domain(s)

| | Analysis | |
|---|---|---|
| Domain description | type | Position(s) on protein |
| Thrombospondin, subtype 1 | FPrintScan | 436-449, 454-465, 473-484 |
| Thrombospondin, type I | HMMPfam | 383-428, 439-489, 496-546 |
| von Willebrand factor, type C | HMMPfam | 318-372 |
| Thrombospondin, type I | HMMSmart | 382-429, 438-490, 495-547 |
| Thrombospondin, N-terminal | HMMSmart | 24-221 |
| von Willebrand factor, type C | HMMSmart | 318-372 |
| Thrombospondin, type I | ProfileScan | 379-429, 435-490, 492-547 |
| von Willebrand factor, type C | ProfileScan | 316-373 |
| von Willebrand factor, type C | ScanRegExp | 336-372 |

Variant protein HUMTHROM_{—}1_P27 (SEQ ID NO:52) is encoded by the transcript HUMTHROM_{—}1_T14 (SEQ ID NO:2). The coding portion of transcript HUMTHROM_{—}1_T14 (SEQ ID NO:2) portion starts at position 326 and ends at position 1990. The transcript also has the following SNPs as listed in Table 26 (given according to their position on the nucleotide sequence, with the alternative nucleic acid listed).

TABLE 26
Nucleic acid SNPs

| SNP position(s) on | Alternative nucleic |
|---|---|
| nucleotide sequence | acid(s) |
| 21 | G -> C |
| 151 | G -> A |
| 188 | T -> C |
| 451 | G -> |
| 560 | G -> A |
| 813 | A -> G |
| 868 | C -> |
| 1035 | G -> A |
| 1311 | A -> G |
| 1615 | G -> A |
| 1735 | C -> T |
| 1757 | G -> |
| 1892 | A -> G |
| 1897 | C -> T |
| 2011 | G -> T |
| 2383 | T -> C |
| 2471 | C -> |
| 2480 | G -> |
| 2551 | A -> G |
| 2627 | C -> |
| 2679 | C -> |
| 2683 | A -> G |
| 3182 | C -> G |
| 3182 | C -> |
| 3452 | T -> C |
| 3457 | G -> |
| 3488 | A -> |
| 3502 | A -> |
| 3555 | A -> G |
| 3613 | G -> C |
| 3844 | C -> |
| 3884 | A -> G |
| 3934 | A -> |
| 4057 | T -> C |
| 4135 | A -> |
| 4176 | C -> T |
| 4375 | G -> A |
| 4402 | T -> A |
| 4442 | A -> T |
| 4511 | C -> A |
| 4595 | A -> T |
| 4682 | G -> A |
| 4760 | C -> G |
| 4773 | C -> T |
| 4808 | A -> C |
| 4985 | T -> G |
| 5073 | T -> |
| 5087 | T -> |
| 5203 | T -> G |
| 5203 | T -> |
| 5227 | T -> |
| 5372 | C -> A |
| 5372 | C -> |
| 5441 | T -> C |
| 5441 | T -> |
| 5557 | T -> C |
| 5558 | C -> T |
| 5626 | T -> |
| 5729 | A -> G |
| 5804 | T -> |
| 5926 | C -> G |
| 6004 | T -> G |
| 6009 | T -> G |
| 6077 | A -> G |

The function of TSP-1 and its splice variants can be examined according to a variety of in vitro and in vivo models. These models examine a variety of different TSP-1 related functions, and examine whether a particular splice variant possesses anti-angiogenic activity.

### Example 3

Validation, Cloning and Expression of TSP-1 Variants

This example relates to the validation, cloning and expression of TSP-1 variants according to the present invention. The following TSP-1 variants were selected: TSP-1_{—}1170 (wt) (SEQ ID NO:54); TSP-1_{—}1112 (SEQ ID NO:56); TSP-1_{—}685 (SEQ ID NO:58); TSP-1_{—}555 (SEQ ID NO:60); TSP-1_{—}173 (positive control) (SEQ ID NO:62).

FIG. 1 provides a schematic drawing of TSP-1 variants of the present invention as well as a known TSP-1 and a previously described P173 anti-angiogenic TSP-1 fragment, also known as the 3TSR fragment (Miao et al. (2001), Cancer Research 61, 7830-7839; Short et al. (2005), J. Cell Biology 168, 643-653). TSP-1 variants of the present invention, depicted in FIG. 1, are TSP-1_{—}1112 (SEQ ID NO:5, 51); TSP-1_{—}685 (SEQ ID NO:4, 50); TSP-1_{—}555 (SEQ ID NO:2, 52), TSP-1_{—}578 (SEQ ID NO:1, 48) and TSP-1_{—}804 (SEQ ID NO:3, 49). All variants include the 3TSR domains that are necessary for activity. Of the five variants, four variants were caused by intron retention and therefore have unique tails. One variant, P1112, is caused by the skipping of the 10^{th} exon. The 3TSR fragment (P173) that was previously shown by Prof. J. Lawler (Miao et al. (2001), Cancer Research 61, 7830-7839; Short et al. (2005), J. Cell Biology 168, 643-653) to exhibit anti-angiogenic activity, and the known WT 1170 variants are shown as well. Exons are represented by orange boxes, while introns are represented by two headed arrows. Proteins are shown in yellow boxes. The unique regions are colored green.The heparin binding domain and the TSR domains are indicated.

Validation of TSP-1_{—}555 variant of the present invention (SEQ ID NO:2):

The expression of TSP-1_{—}555 and TSP_{—}578 variants was validated at the mRNA level. The TSP-1_{—}555 transcript was validated using cDNA prepared from RNA mix extracted from heart and brain tissues (Ichilov); bone cell line (SaOs-2-#ATCC HTB-85) and fibroblasts cell line (BJ # ATCC CRL2522). The experimental method used was as follows.

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8). The table 66 below shows the sequences of the primers used for the PCR reaction of TSP 555 (SEQ ID NO: 2), while table 67 shows the sequences of PCR pprimers used for the PCR reaction of TSP578 (SEQ ID NO: 1). Orientation for the primers is given as F (forward) or R (reverse).

TABLE 66

| | | Nucleotide |
|---|---|---|
| | | coordinates |
| | | on target |
| | | sequence |
| Oligonucleotide sequence | | (SEQ |
| (ID) | Orientation | ID NO: 2): |
| 5′ GCTCCTGCGATAGCCTCAAC-3′ | F | 1536 |
| (100-350_F_TSP-1_T17_N23) SEQ ID | | |
| No: 63 | | |
| 5′-CAAATCGCTCAGGACTAACC-3′ | R | 2077 |
| (100-353_R_TSP-1_T14_N30) SEQ ID | | |
| NO: 64 | | |

TABLE 67

| | | Nucleotide |
|---|---|---|
| | | coordinates |
| | | on target |
| | | sequence |
| Oligonucleotide sequence | | (SEQ |
| (ID) | Orientation | ID NO: 1): |
| 5′ TGATAGCTGCACTGAGTGTC-3′ | F | 1324 |
| (100-346_F_TSP-1_T12_N16) (SEQ ID | | |
| NO: 120) | | |
| 5′-CTCTATGACCCACTGAACTG-3′ | R | 1892 |
| (100-347_R_TSP-1_T12_N28) (SEQ ID | | |
| NO: 121) | | |

PCR amplification and analysis

cDNA (5ul), prepared as described above (RT PCR), was used as a template in PCR reactions. The amplification was done using AccuPower PCR PreMix (Bioneer, Korea, Cat# K2016), under the following conditions: lul—of each primer (10 uM) plus 13 ul—H₂O were added into AccuPower PCR PreMix tube with a reaction program of 5 minutes at 94° C.; 35 cycles of: [30 seconds at 94° C., 30 seconds at 55° C. 60 seconds at 72° C] and 10 minutes at 72° C. At the end of the PCR amplification, products were analyzed on agarose gels stained with ethidium bromide and visualized with UV light. The PCR products were extracted from the gel using QiaQuickTM gel extraction kit (Qiagen, Cat #28706). The extracted DNA products were sequenced by direct sequencing using the gene specific primers described above (Hy-Labs, Israel).

The PCR products sequence for TSP 555 (SEQ ID NO:122) and TSP 578 (SEQ ID NO:123) are given below. The primers sequence is underlined.

PCR product for TSP 555
GCTCCTGCGATAGCCTCAACAACCGATGTGAGGGCTCCTCGGTCCAGACA
CGGACCTGCCACATTCAGGAGTGTGACAAGAGATTTAAACAGGATGGTGG
CTGGAGCCACTGGTCCCCGTGGTCATCTTGTTCTGTGACATGTGGTGATG
GTGTGATCACAAGGATCCGGCTCTGCAACTCTCCCAGCCCCCAGATGAAC
GGGAAACCCTGTGAAGGCGAAGCGCGGGAGACCAAAGCCTGCAAGAAAGA
CGCCTGCCCCATCAATGGAGGCTGGGGTCCTTGGTCACCATGGGACATCT
GTTCTGTCACCTGTGGAGGAGGGGTACAGAAACGTAGTCGTCTCTGCAAC
AACCCCACACCCCAGTTTGGAGGCAAGGACTGCGTTGGTGATGTAACAGA
AAACCAGATCTGCAACAAGCAGGACTGTCCAATTGGTGAGCCACGCAGCC
CAGGATGAAACGACCCAGGAGCTTTGCTCTTTTACTGAATGCTGCAGTCA
GCATTCGAGGAGATTCCAGCTTGGTTAGTCCTGAGCGATTTG

PCR product for TSP 578
TGATAGCTGCACTGAGTGTCACTGTCAGAACTCAGTTACCATCTGCAAAA
AGGTGTCCTGCCCCATCATGCCCTGCTCCAATGCCACAGTTCCTGATGGA
GAATGCTGTCCTCGCTGTTGGCCCAGCGACTCTGCGGACGATGGCTGGTC
TCCATGGTCCGAGTGGACCTCCTGTTCTACGAGCTGTGGCAATGGAATTC
AGCAGCGCGGCCGCTCCTGCGATAGCCTCAACAACCGATGTGAGGGCTCC
TCGGTCCAGACACGGACCTGCCACATTCAGGAGTGTGACAAGAGATTTAA
ACAGGATGGTGGCTGGAGCCACTGGTCCCCGTGGTCATCTTGTTCTGTGA
CATGTGGTGATGGTGTGATCACAAGGATCCGGCTCTGCAACTCTCCCAGC
CCCCAGATGAACGGGAAACCCTGTGAAGGCGAAGCGCGGGAGACCAAAGC
CTGCAAGAAAGACGCCTGCCCCAGTAAGTGTGAGGTCCGCTGCAAGGGTG
AGCATGGGCAGCAGCTCTGCCCAGCTGGTTGCCTGGCATCTGCAGCCTGC
AGTTCAGTGGGTCATAGAG

Cloning

The nucleotide sequences of all of the TSP-1 variants were codon optimized to boost protein expression in a mammalian system. The optimized sequences were synthesized by BlueHeron (USA) by using their proprietary gene synthesis technology with the addition of sequence encoding the StrepII and His tags at the 3′.

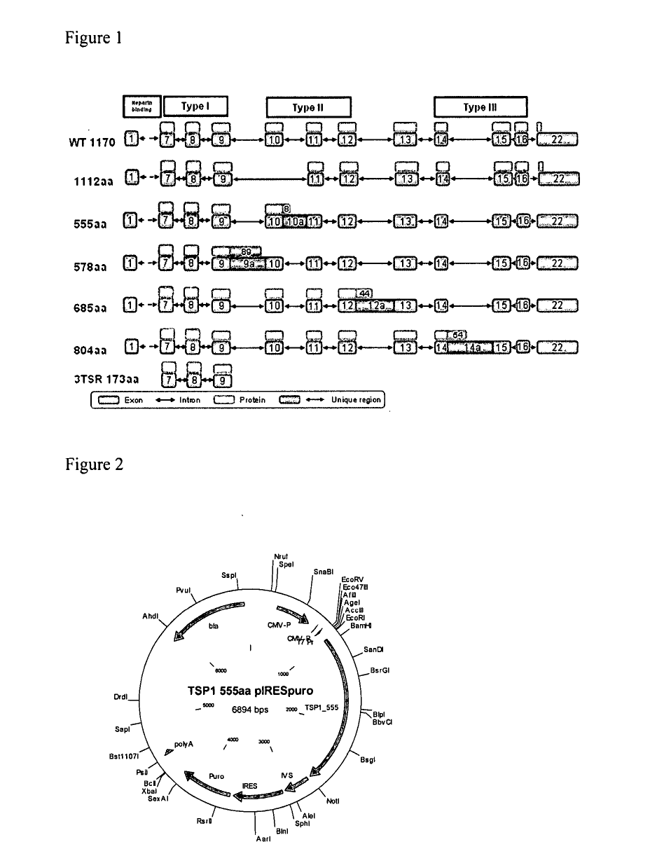

The optimized sequences were cloned into EcoRI-Notl sites of plRESpuro3 expression vector. An exemplary, illustrative non-limiting plasmid, suitable for use with the present invention, is the pIRESpuro3 vector. FIG. 2 shows a schematic map of TSP-1_{—}555 in the pIRESpuro3 vector.

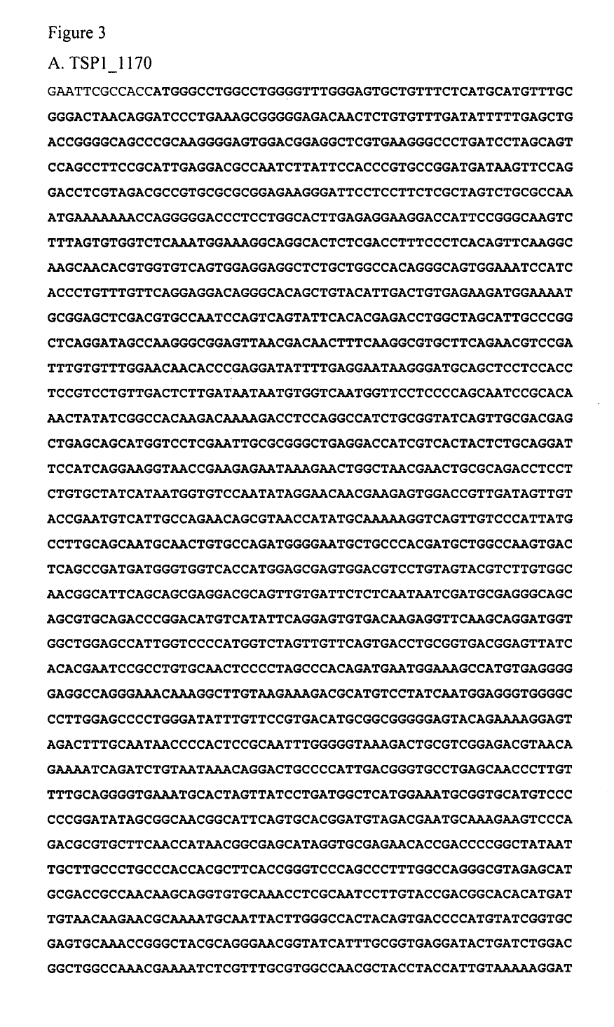

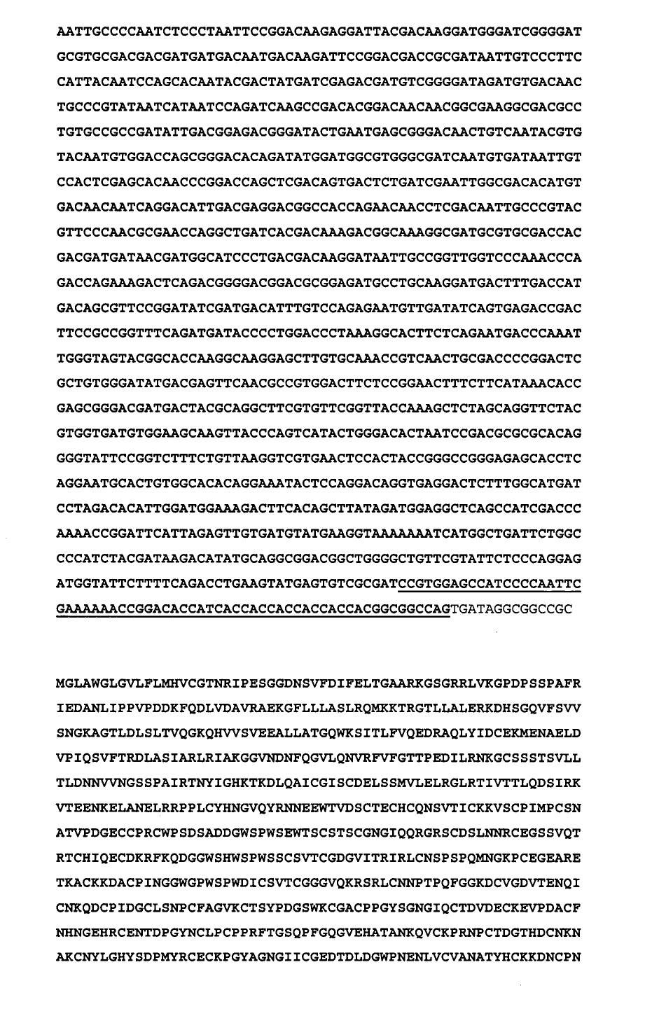

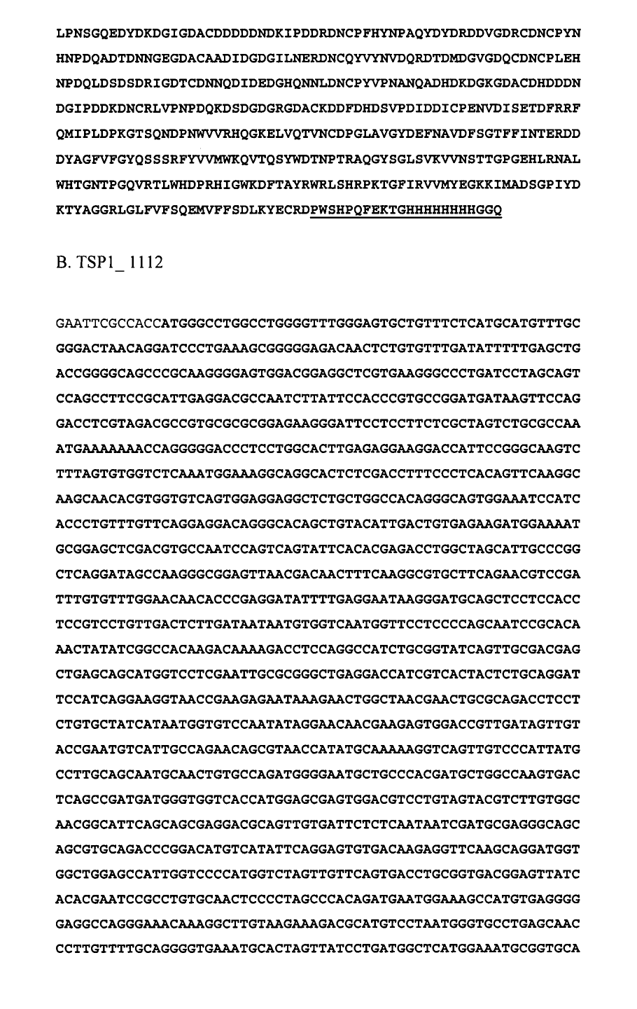

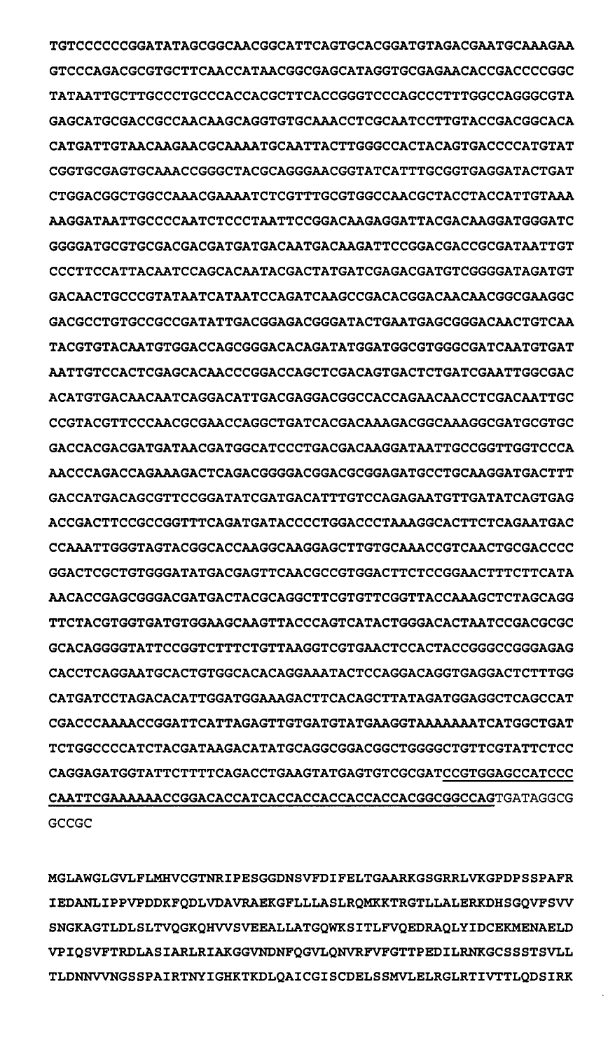

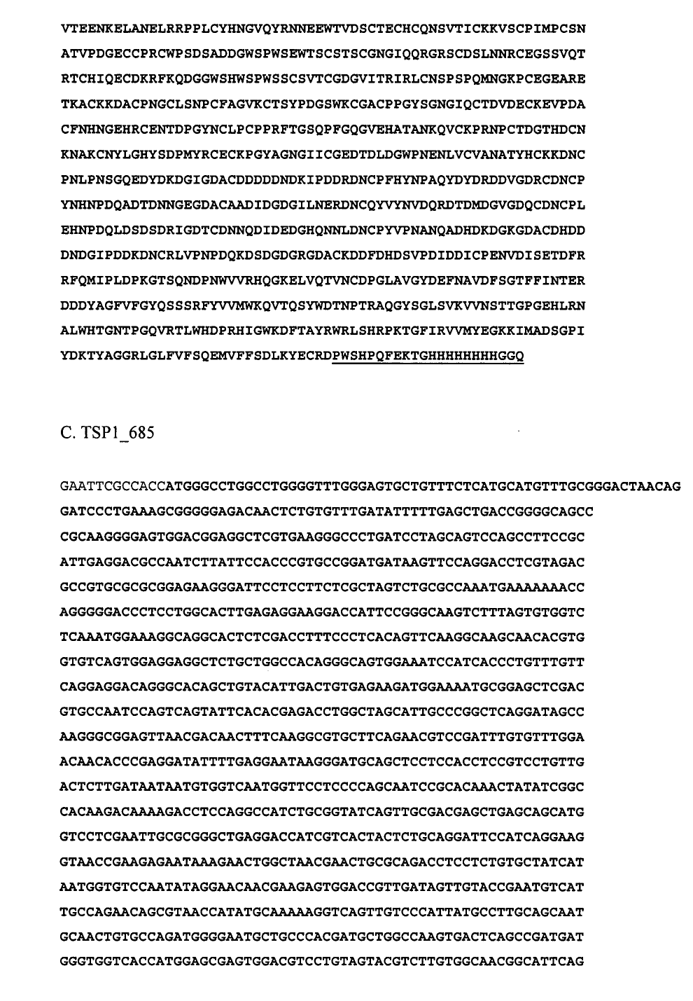

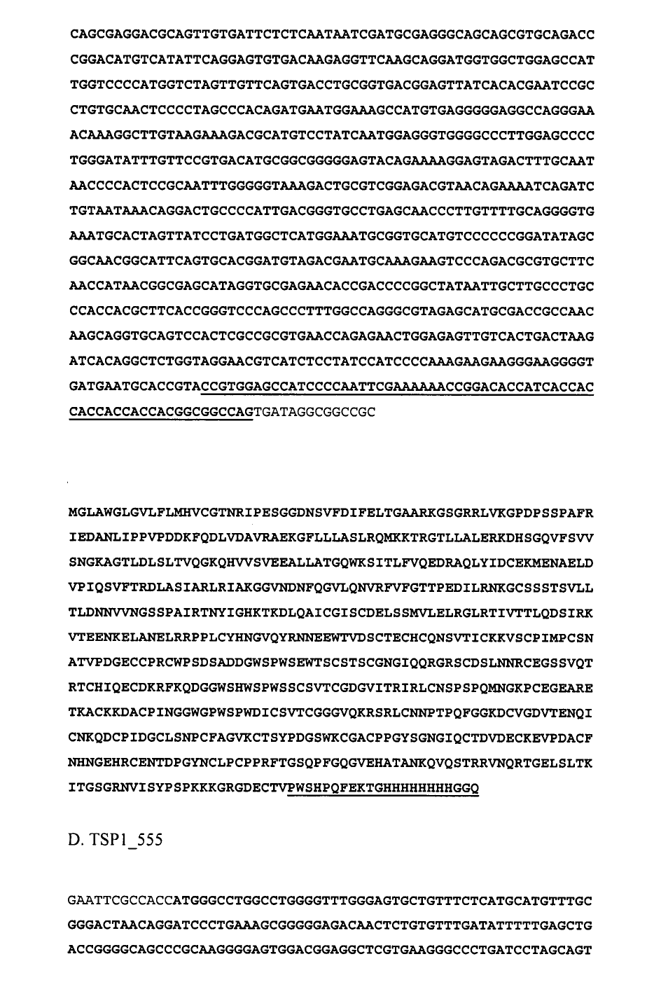

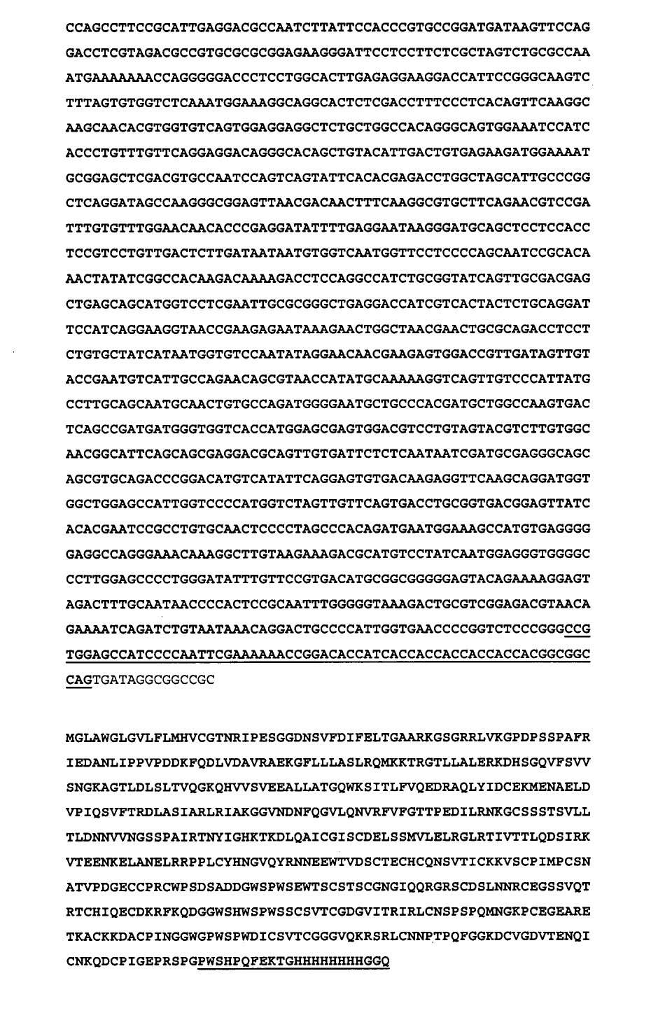

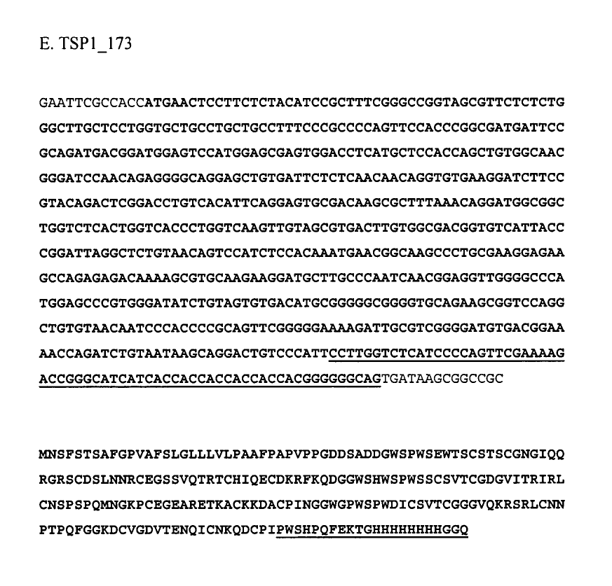

The optimized cloned sequences of all the TSP-1 variants, containing the Strep-His tag, are given in FIG. 3. The relevant ORFs (open reading frames) including the tag sequences are shown in bold; StrepHis tag sequences are underlined. FIG. 3A demonstrates the nucleic acid (SEQ ID NO: 53) and the amino acid sequence (SEQ ID NO:54) of TSP-1-1170; FIG. 3B demonstrates the nucleic acid (SEQ ID NO:55) and the amino acid (SEQ ID NO:56) sequence of TSP-1-1112; FIG. 3C demonstrates the nucleic acid (SEQ ID NO:57) and the amino acid (SEQ ID NO:58) sequence of TSP-1-685; FIG. 3D demonstrates the nucleic acid (SEQ ID NO:59) and the amino acid (SEQ ID NO:60) sequence of TSP-1-555; FIG. 3E demonstrates the nucleic acid (SEQ ID NO:61) and the amino acid (SEQ ID NO:62) sequence of TSP-1-173.

Transfection of TSP-1 Constructs

The TSP-1 constructs were transfected into HEK-293T cells (ATCC # CRL-11268) as follows. One day prior to transfection, one well from a 6 well plate was plated with 500,000 cells in 2 ml DMEM. On the day of transfection, the FuGENE 6 Transfection Reagent (Roche, Cat#: 1-814-443) was warmed to ambient temperature and mixed prior to use. 6 μl of FuGENE Reagent were diluted into 100 μl DMEM (Dulbecco's modified Eagle's medium; Biological Industries, Cat#: 01-055-1A). Next, 2 micrograms of construct DNA were added. The contents were gently mixed and incubated at room temperature (RT) for 15 minutes. 100 μl of the complex mixture was added dropwise to the cells and swirled. The cells were incubated overnight at 37° C. with 5% CO2. Following about 48 h, transfected cells were split and subjected to antibiotic selection with 5 microgram/ml puromycin. The surviving cells were propagated for about three weeks.

Expression Analysis

The supernatants of the TSP-1 puromycin resistant cells were bound to NiNTA beads as follows: for each sample, 50ul Ni-NTA agarose (Qiagen #1018244) were washed twice with water and ×2 with ×1 IMIDAZOLE buffer (Biologicals industries #01-914-5A) and then centrifuged for 5 min at 950× g. 1 ml of cell supernatant was added to the beads and the samples were gently shaken for 45 min. at RT. Then, the samples were spun down and washed with ×1 IMIDAZOLE buffer, and centrifuged again at 950× g for 5 min. The samples were eluted with 50 ul SDS sample buffer, incubated for 5 min. at 100° C. and loaded on a 12% SDS-PAGE gel.

Following electrophoresis, proteins on the gel were transferred to nitrocellulose membranes for 60 min at 35 V using Invitrogen's transfer buffer and X-Cell II blot module. Following transfer, the blots were blocked with 5% skim milk in wash buffer (0.05% Tween-20 in PBS) for at least 60 min. at room temperature with shaking. Following blocking, the blots were incubated for 60 min at room temperature with a commercially available mouse anti Histidine Tag (Serotec, Cat# MCA1396) and diluted in 1/5 blocking buffer, followed by washing with wash buffer and incubation with the secondary antibody Goat anti Mouse HRP (Jackson, Cat# 115-035-146) and diluted 1:25,000 in 1/5 blocking buffer. Next, ECL (Enhanced Chemiluminescence) detection was performed according to the manufacturer's instructions (Amersham; Cat # RPN2209).

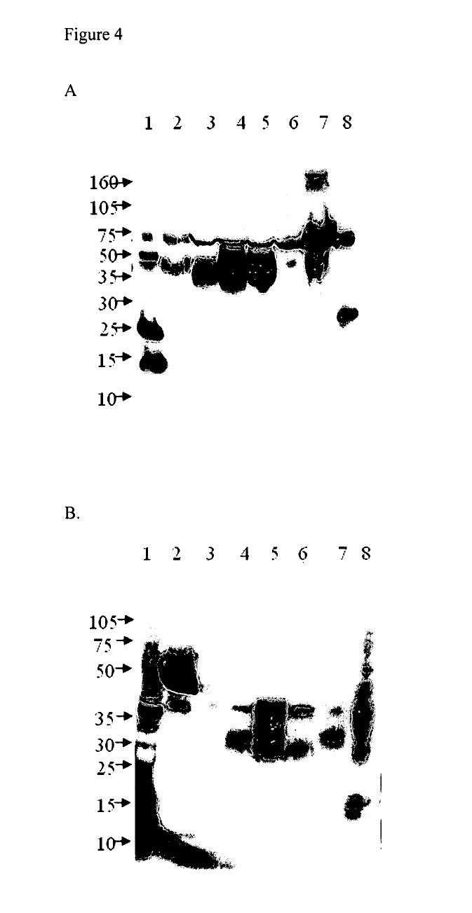

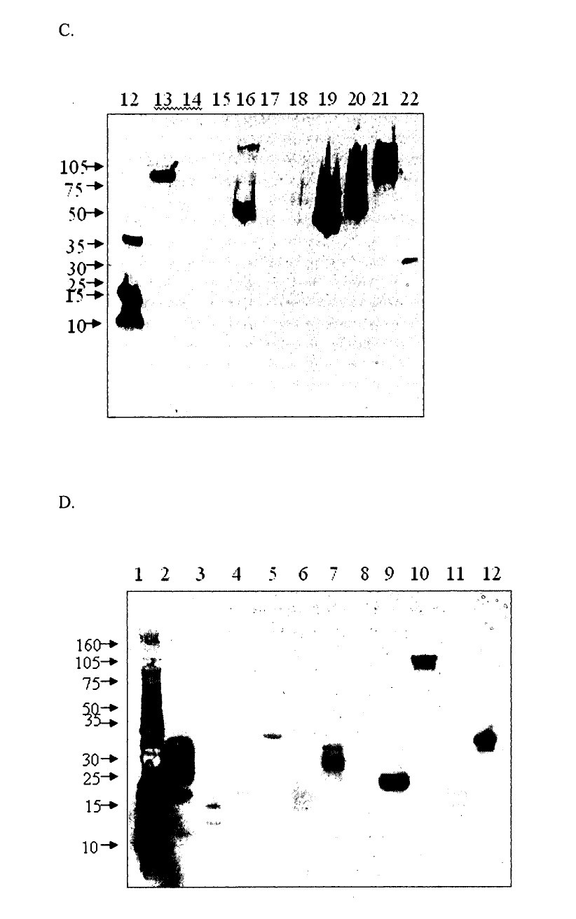

The results, demonstrating stable TSP-1 expression, are shown in FIG. 4. FIG. 4A lane 5 represents the expression of TSP-1_{—}173 (3TSR) (SEQ ID NO:62); lane 7 represents TSP-1_{—}555 (SEQ ID NO:60); lane 1 represents the molecular weight marker (Rainbow Amersham RPN800); lane 2 represents mock plRESpuro3 (also referred to herein as “mock”, or cells that were transfected with the vector alone, without any variant or known TSP-1 sequence); and lane 8 represents Strep-His control (˜100 ng). FIG. 4B lane 2 represents the expression of TSP-1_{—}685 (SEQ ID NO:58); lane 1 represents molecular weight marker (Rainbow Amersham RPN800); and lane 8 represents Strep-His control (˜100 ng). FIG. 4C lane 13 represents the expression of TSP-1_{—}1170 (SEQ ID NO:54); lane 12 represents molecular weight marker (Rainbow Amersham RPN800); lane 22 represents Strep-His control (˜100 ng). FIG. 4D lane 10 represents the expression of TSP-1_{—}1112 (SEQ ID NO:56); lane 1 represents molecular weight marker (Rainbow Amersham RPN800); and lane 12 represents Strep-His control (˜100 ng).

### Example 4

TSP-1 Variant Protein Production and Purification

Production:

TSP-1 wild type TSP-1 1170 (SEQ ID NO:54), the positive control TSP-1 173 (SEQ ID NO:62) (3TSR=173) and 3 TSP-1 variants of the present invention were produced in HEK293T cells, all StrepII-His-tagged at their C-termini. In addition, the IL6 signal peptide was added to the positive control TSP-1 173.

TSP-1 variants according to the present invention were produced using IMDM containing CaCl₂ at a final concentration of 2.5 mM in “Cell Factory” units (Nunc, Cat# 164327). This methodology was selected since several TSP-1 variants of the present invention include calcium binding sites. As reported in the literature, the conformational integrity of thrombospondins depends on binding of calcium ions; therefore, the production and purification protocols were adapted accordingly, and the TSP-1 proteins were produced and purified in the presence of 2.5 mM calcium.

Cells expressing TSP-1 1170 (SEQ ID NO:54) and TSP-1 555 (SEQ ID NO:60) were harvested once, after 4 days of incubation, resulting in 2L harvest for each protein.

Cells expressing TSP-1 1112 (SEQ ID NO:56), TSP-1 173 (SEQ ID NO:62) and TSP-1 685 (SEQ ID NO:58) were harvested twice: after 4 and 6 days of propagation, resulting in 2L harvest each time for each protein. All harvest batches were centrifuged, filtered through a 0.22 um filter and used for protein purification.

Purification

The protein purification protocol was performed as follows. For purification of proteins featuring the His Strep tag, proteins were purified by affinity chromatography using Ni-NTA (nickel-nitrilotriacetic acid) resin. This type of chromatography is based on the interaction between a transition Ni²+ion immobilized on a matrix and the histidine side chains of His-tagged proteins. His-tag fusion proteins can be eluted from the matrix by adding free imidazole for example, as described below. The purification method preferably uses the StrepII/8xHistidine system (double-tag) to ensure purification of recombinant proteins at high purity under standardized conditions. A protein according to the present invention, carrying the 8xHistidine-tag and the Strep-tag II at the C—terminus, can be initially purified by IMAC (Immobilized metal ion affinity chromatography) based on the 8xHistidine-tag-Ni-NTA interaction. After elution from the Ni-NTA matrix with imidazole, the protein (which also carries the Strep-tag II epitope) can be loaded directly onto a Strep-Tactin matrix. No buffer exchange is required. After a short washing step, the recombinant protein can be eluted from the Strep-Tactin matrix using desthiobiotin.

More specifically with regard to the actual process that was performed, His-tag labeled proteins according to the present invention were purified by affinity chromatography using Ni-NTA resin, according to the following protocol. The supematent was prepared as previously described and transferred to 3×250 ml centrifuge tubes. Six ml of Ni-NTA Superflow beads (Ni-NTA Superflow®, QIAGEN) were equilibrated with 10 column volumes of WFI (Teva Medical #AWF7114) and 10 column volumes of Buffer A (20 mM Tris, 2 mM CaCl₂, 300 mM NaCl, 10 mM imidazole, pH 8.0). The beads were added to the filtered supernatant, and the tube was incubated overnight on a rocking platform at 4° C. The Ni-NTA beads in the 3×250 ml centrifuge tube were separated from the supernatant and packed in a 6 ml column of Ni-NTA Superflow. Beads were washed with buffer A at a flow rate of 1 column volume per minute, until O.D280 nm was lower than 0.01 mAU.

Next, 1 ml Strep-Tactin Superflow beads were equilibrated with 10 CVs (column volumes) of WFI (Teva Medical #AWF7114) and 10 column volumes of Buffer A (20 mM Tris, 2 mM CaCl₂, 300 mM NaCl, 10 mM imidazole, pH 8.0). The protein was eluted from the-Ni-NTA beads with buffer B (20 mM Tris, 2 mM CaCl₂, 300 mM NaCl, 250 mM imidazole, pH 8.0) at a flow rate not higher than 1 ml/min and was then placed on the Step-Tactin column. Once the protein was washed from the Ni-NTA beads, the column was disconnected. The Strep-Tactin column was then washed with Buffer A, at a flow rate of 1 CV/min, with at least 5 CVs, until O.D280 nm was less then 0.01 mAU. The protein was eluted from the Strep-Tactin column with Strep-Tactin Elution Buffer (Buffer C; 20 mM Tris, 2 mM CaCl₂, 300 mM NaCl, 10 mM imidazole, 2.5 mM desthiobiotin, pH 8.0) at 0.2 ml per minute. Imidazole was removed from the purified protein by dialysis against Tris buffered saline (20 mM Tris-Cl pH 7.4, 150 mM NaCl) supplemented with 2 mM CaCl₂ for half of the purified protein product, and in DMEM Medium, for the other half, both at 4° C.

Product Analysis

Purified TSP-1 variants according to the present invention were subjected to LC-MS/MS (mass spectrometry) to confirm sample identity. Bands were cut from a Coomassie gel (not shown) and samples were sent to the Technion Proteomic Center for MS-MS identification. The identity of all proteins was confirmed.

The Molecular Weight (MW), concentration and purity of the final product were analyzed by Bioanalyser according to manufacturer instructions, and are shown in Table 68 below.

TABLE 68

| | | Concentration | |
|---|---|---|---|
| Variant | Peak no. | ug/ml | Purity % |
| TSP-1 1170 | 15 (DMEM) | 2749.3 | 88.3 |
| TSP-1 685 | Not visible | 350 (gel) | 80 (gel) |
| TSP-1 1112 | 14 (TBS) | 1526.5 | 90.9 |
| | 13 (DMEM) | 1144 | 78.8 |
| TSP-1 555 | 9, 10 (TBS) | 716 | 87.3 |
| | 8 (DMEM) | 509 | 91.2 |
| TSP-1 173 | 9 (TBS) | 1184 | 85.3 |
| | 7 (DMEM) | 877 | 90.8 |

### Example 5

Activity of TSP-1 Variants

In Vitro Models

This Example relates to functional testing of TSP-1 variants according to the present invention, produced as described above. As described in greater detail below, the TSP-1 variants according to the present invention inhibited VEGF-induced migration of HDMEC (human dermal microvascular endothelial cells).

Inhibition of Endothelial Cell Migration

In vitro biological activity of TSP-1 variants of the present invention was assessed in a VEGF-induced migration assay of HDMECs, which is a known in vitro surrogate assay for the inhibition of angiogenesis in vivo. The inhibitory activity of TSP-1 variants of the present invention was compared to that of purified human platelet TSP-1, and in house produced WT TSP-1 (TSP-1-1170) and 3TSR domain (TSP-1-173) as positive controls. TSP-1

Endothelial cell migration was performed for 4 hrs with Vitrogen-coated membranes in transwell plates, in the presence of 30 ng/ml VEGF in the bottom wells, while the inhibitory proteins with the cells were placed in the top wells. The cells that migrated to the bottom of the membrane were stained and counted. The endothelial cell migration assay was carried out as follows:

Two wells per parameter were used (Costar Transwell Plates, cat #3422).

The coating was performed as follows: both sides of the membrane were coated with 10 μg/ml of Vitrogen (Vitrogen 100, Cohesion Technologies, FXP-019, in PBS). The bottom of the membrane was coated first by flipping inserts upside down on lid. Afterward, 40 μl of Vitrogen were placed on the membrane and incubated for 20 minutes in a tissue culture hood, and then placed back in a 24 well tissue culture plate by placing the plate on top of the inserts and flipping the plate back up, thereby minimizing disturbances to the membrane coating. At this point, 50 μl of Vitrogen were added to the top of the membrane and were incubated overnight at 4° C. Blocking of the membrane was done with 5% BSA/PBS (Sigma, A7906) for one hour at room temperature in the tissue culture hood by adding 500 μl to the bottom well to block the bottom of the membrane, and 100 μl inside the well to block the top of the membrane. Afterward, the blocking media was removed, and the bottom and top of membranes were washed with the same volume of PBS. While the cells were incubating with proteins, the membranes were kept with PBS. A primary line of human dermal microvascular endothelial cells (HDMECs) were grown in DMEM (Mediatech, MT 10-013-CV) with 10% FCS (Mediatech MT 35-015-CV). The day before the migration, low serum media (2% FBS) was added to the endothelial cells overnight. To harvest the cells, the following process was performed: trypsinization for 5 minutes, washing with DMEM with 10% FCS. The cells were counted using a hemacytometer (the required cell density was about 10⁵ cells per well, and twice that per parameter). The cells were spun down and resuspended in 5 mls of DMEM/BSA media. The endothelial cells were divided into 1.5 ml Eppendorf tubes, and spun at 3×10⁴ RPM in microcentrifuge.

Cells were resuspended in DMEM/2% BSA with 0.2, 2 or 20 nM of variant proteins. PBS was removed from the top and bottom wells. To the bottom well, 750 μl of DMEM/2%BSANVEGF (30 ng/ml) were added. Then the cells were added to the top wells and placed in a tissue culture incubator (37° C.) for 4 hrs.

The inserts were placed in empty wells, and using Q-tips cells were removed from the top of the membrane. Next, 30 μl PBS were added to wash the membrane, following with additional wiping with a Q-tip. Each insert was placed in a well containing 1 ml of 0.2% crystal violet (Sigma, C3880) in 2% ethanol for 15 minutes, followed by a quick wash in a well filled with water. The slides were labeled and two to three dots of oil were placed on each slide. The filter was placed bottom side up, and the membrane was cut out using a razor blade. The membrane was then placed carefully on oil on the slide and coverslips were placed on the membranes. One side of each coverslip was sealed with fingernail polish. The number of cells (purple nuclei) was counted in 20× or 40× field, four fields per filter.

Results:

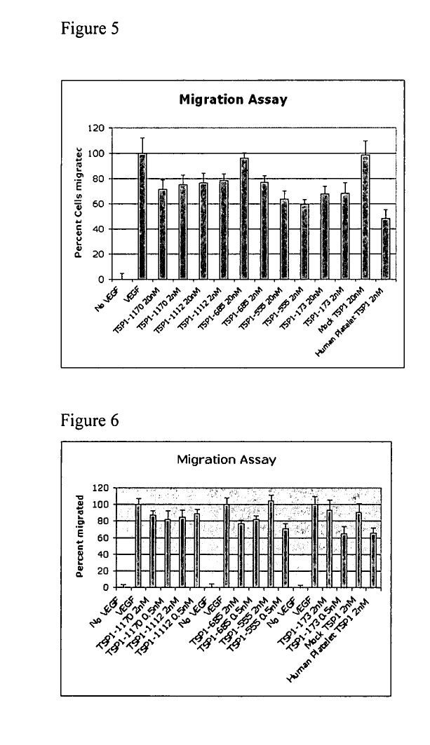

Two assays were performed with each of the TSP-1 variant proteins. The concentrations of the proteins in the first assay were 2 nM and 20 nM, and in the second assay they were 0.5 nM and 2 nM. The results of the first assay are shown in FIG. 5, and the results of the second assay are shown in FIG. 6. The histograms depict the percentage of cells that migrated, where the number of cells that migrated in response to VEGF (in the absence of inhibitory proteins) is defined as 100%, and the number observed in the absence of VEGF is defined as 0%. The raw cell counts indicate that there is a 4 to 7-fold difference in these two values, in the different plates (a 2-fold or greater difference indicates that the cells are responding well to VEGF). The controls worked well in that the mock had no effect and the human platelet TSP-1 and the TSP-1-173 controls inhibited by about 30-50%. Most of the TSP-1 variants of the present invention showed inhibitory activity. The shortest variant of the present invention, TSP-1-555, had the most activity, which was similar to that of the positive controls (for some unknown reason, both TSP-1-555 and TSP-1-173 inhibited at 0.5 nM but not at 2 nM in the second experiment; this may simply be due to some problem with the experiment itself).

The results indicate that the proteins of TSP-1-685 and TSP-1-555 variants of the present invention have significant inhibitory activity in the migration assay. The level of activity for TSP-1-555 is similar to that of the control TSP-1-173, and approaches that of human platelet known or WT TSP-1. In these assays, TSP-1-685 also significantly inhibited cell migration, but appeared somewhat less active than TSP-1-555.

Competition Binding of Labeled TSP-1 Variant and Known (WT) TSP-1

Tritium-labeled TSP-1 variant (1 nM) and various concentrations of known or WT TSP-1 (0-20 nM) are added to eppendorf tubes each containing 100 000 HMVEC cells that are grown in full media and scraped from T175 flasks at about 80% confluency. The tubes are mixed and incubated for 2 h on ice. The number of counts remaining bound to the cells after extensive washing determines total amount of the variant which is bound (the experiment may optionally be performed in reverse, in which the known or WT TSP-1 is labeled and TSP-1 variant is added as cold, non-labeled competitor). The Kd of tritium-labeled protein is preferably previously determined from saturation binding experiments. A competitive binding experiment shows similar Kd values for variant and known TSP-1.

Effect of TSP-1 Variant on Cell Apoptosis

The effect of TSP-1 variants according to the present invention on cell apoptosis is preferably determined by examining human endothelial cells, such as HUAEC cells, with a histone ELISA apoptosis assay (Roche, Indianapolis, Ind.). Five thousand cells per well are plated in 96-well CoStar tissue culture plates. Cells are allowed to adhere, and the variants are added to the wells in an appropriate solution and are incubated overnight. Apoptosis is determined from triplicate samples, and the apoptotic index is determined as a ratio of absorbance of treated cells over absorbance of untreated cells. Other apoptosis assays could be used as well.

In Vivo Models

Aortic Ring Assay of Angiogenesis Ex Vivo

This assay enables the assessment of the effect of TSP-1 variants, according to the present invention, in an ex vivo vascular sprouting experiment, in which the effect of the variants on angiogenesis is tested on rings sliced from the aorta of mice or rats.

Briefly, aortas are taken from mice or rats, cleaned of fat, clotted blood and debris. About 1 mm rings are prepared and embedded individually in 24-well plates, in growth factor-reduced matrigel (in the presence or alternatively in the absence of VEGF) (0.5 ml per well). The matrigel solution is prepared in culture medium (0.5 ml of M199 +FCS) containing either the experimental compounds at various concentrations (in 4 replicates) or controls. Finally, culture medium containing similar concentrations of test reagents is applied to each well. Plates are stored at 37° C. Culture media is changed every 48 hrs. After an incubation period, of about 7 days, the aortic rings are fixed with a formalin solution. The radial lengths of the vascular sprouts of each ring are quantitated from digital images.

Rat Cornea Model of Angiogenesis in Vivo

This model enables to examine the effect of TSP-1 variants, according to the present invention, to be tested in a controlled in vivo experiment, in which the effect of the variants on angiogenesis is localized to a particular portion of the animal's anatomy, in this case the cornea.

Briefly, both corneas of anesthesized rats are implanted on day 0 with a hydron pellet containing sucralfate mixed with either vehicle alone, bFGF or VEGF, in the presence or absence of TSP-1 variants. Alternatively, the TSP-1 variants are added systemically by daily i.p. injections. At an appropriate time point, preferably days 5 to 7 days after implantation, the corneas of the rat eyes are examined by slit-lamp microscopy. An image analysis system is preferably used to record the image and to measure the degree of neovascularization. The results are expressed as the mean of vessel density and are preferably an average of the readings from five corneas per dose.

Matrigel Plug Model of Angiogenesis in Vivo

Mice are injected subcutaneously with Matrigel containing VEGF165 and/or bFGF. Beginning Iday after implantation, mice are injected i.p. daily or 3× weekly with the EphA2 variant. Alternatively, the protein can be delivered continuously by osmotic minipums (Alzet Corporation), implanted subcutaneously. After 7 to 10 days, mice are sacrificed and Matrigel plugs are removed. Neovascularization can be assessed by staining for CD31 (an endothelial marker) and analysis of microvascular vessel density and length. Alternatively, neovascularization can be assessed by analysis of hemoglobin content in the Matrigel plugs.

Human Cancer Model: Establishment of Xenografts in Immune-Deficient Mice.

Human cancer cells, such as human bladder cancer cells (253J B-V), human breast cancer cells (MDA-MB-435) or human pancreatic cells (AsPC-1), are implanted orthotopically or subcutaneously into the legs' flank of immune-deficient mice. Other human xenograft cancer models could also be used. About 5-7 days postimplantation, mice are inoculated introperitoneally daily or 3× weekly with the TSP-1 protein variant. Alternatively, mini osmotic pumps can be used for continuous delivery of the protein. Tumor volumes are determined by caliper measurements every 3-4 days. After 3 to 5 weeks, tumors are excised, weighed and measured. Frozen tumor sections are prepared and immunohistochemistry is carried out for CD31 staining of vascularization and for TUNEL staining of apoptotic cells. Tumor-associated microvessel density and endothelial cell apoptosis are quantified using image software analysis.

Syngeneic Cancer Models- Primary and Metastatic Tumors.

A highly metastatic syngeneic murine cancer model involves injection of murine melanoma cells B16F10 or Lewis lung carcinoma cells into the tail vein of C57/B16 mice. On day 2 postimplantation, systemic therapy begins by daily intraperitoneal injections or osmotic pump delivery of the TSP-1 variant proteins. After 3 weeks, animals are sacrificed and the lungs are harvested, weighed and fixed. The metastases visible on the surface of excised lungs are counted. Alternatively, these cells can be injected subcutaneously on the back of each mouse. Tumors are then measured with a dial caliper.

In Vivo Model of Retinal Neovascularization: Retinopathy of Prematurity in Rats.

Retinal neovascularization is induced in newborn rats, by placing them and their mother in an oxygen chamber with oxygen concentration alternating between 50 and 10% every 24 hrs for 14 days, mimicking conditions in premature infants. On day 14, animals are removed into room air. Control or test proteins (TSP-1 variants) are injected intravitreally at day 14/0 or 14/2 and abnormal neovascularization is assessed on day 14/6. Retinas are dissected and stained for ADPase activity, a procedure that preferentially stains retinal vascular endothelium and microglia in rats of this age. Neovascularization can be assessed by imaging software, or a semiquantitative assessment of severity of vascular disease can be carried out by independent examiners in a blinded fashion.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An isolated polynucleotide consisting of the transcript selected from the group consisting of HUMTHROM_{—}1_T12 (SEQ ID NO:1), HUMTHROM_{—}1_T14 (SEQ ID NO:2), HUMTHROM_{—}1_T15 (SEQ ID NO:3), HUMTHROM_{—}1_T17 (SEQ ID NO:4), HUMTHUROM_{—}1_T32 (SEQ ID NO:5), or the polynucleotide at least about 95% homologous thereto.

2. An isolated polypeptide consisting of the protein variant selected from the group consisting of HUMTHROM_{—}1_P8 (SEQ ID NO:48), HUMTHROM_{—}1_P10 (SEQ ID NO:49), HUMTHROM_{—}1_P12 (SEQ ID NO:50), HUMTHROM_{—}1_P22 (SEQ ID NO:51), HUMTHROM_{—}1_P27 (SEQ ID NO:52), or the polypeptide at least about 95% homologous thereto.

3. An isolated chimeric polypeptide consisting of a first amino acid sequence being at least 95% homologous to amino acids 1-751 of TSP-1_HUMAN_V1 (SEQ ID NO:47), which also corresponds to amino acids 1-751 of HUMTHROM_{—}1_P10 (SEQ ID NO:49), and a second amino acid sequence being at least 95% homologous to a polypeptide having the sequence VKTVFYPFFIFSVQQQPETLWDSRKLHGYSKKYTKSIHRIIRNYSLCSSS LRM corresponding to amino acids 752-804 of HUMTHROM_{—}1_P10 (SEQ ID NO:49), wherein said first amino acid sequence and second amino acid sequence are contiguous and in a sequential order.

4. An isolated polypeptide consisting of the amino acid sequence being at least at least about 95% homologous to the sequence VKTVFYPFFIFSVQQQPETLWDSRKLHGYSKKYTKSIHRIIRNYSLCSSS LRM of HUMTHROM_{—}1_P10 (SEQ ID NO:49).

5. An isolated chimeric polypeptide consisting of a first amino acid sequence being at least about 95% homologous to amino acids 1-643 of TSP-1_HUMAN_V1 (SEQ ID NO:47), which also corresponds to amino acids 1-643 of HUMTHROM_{—}1_P12 (SEQ ID NO:50), and a second amino acid sequence being at least about 95% homologous to a polypeptide having the sequence QSTRRVNQRTGELSLTKITGSGRNVISYPSPKKKGRGDECTV corresponding to amino acids 644-685 of HUMTHROM_{—}1_P12 (SEQ ID NO:50), wherein said first amino acid sequence and second amino acid sequence are contiguous and in a sequential order.

6. An isolated polypeptide consisting of the amino acid sequence being at least about 95% homologous to the sequence QSTRRVNQRTGELSLTKITGSGRNVISYPSPKKKGRGDECTV of HUMTHROM_{—}1_P12 (SEQ ID NO:50).

7. An isolated chimeric polypeptide consisting of a first amino acid sequence being at least about 95% homologous to amino acids 1-490 of TSP-1_HUMAN_V1 (SEQ ID NO:47), which also corresponds to amino acids 1-490 of HUMTHROM_{—}1_P22 (SEQ ID NO:51), a second bridging amino acid sequence comprising of N, and a third amino acid sequence being at least about 95% homologous to to amino acids 550-1170 of TSP-1_HUMAN_V1 (SEQ ID NO:47), which also corresponds to amino acids 492-1112 of HUMTHROM_{—}1_P22 (SEQ ID NO:51), wherein said first amino acid sequence, second amino acid sequence and third amino acid sequence are contiguous and in a sequential order.

8. An isolated polypeptide consisting of the polypeptide having a length “n”, wherein n is about 10 amino acids in length, wherein at least three amino acids comprise PNG having a structure as follows (numbering according to HUMTHROM_{—}1_P22 (SEQ ID NO:51)): a sequence starting from any of amino acid numbers 490−x to 490; and ending at any of amino acid numbers 492+((n−2)−x), in which x varies from 0 to n−2.

9. An isolated polypeptide consisting of the polypeptide having a length “n”, wherein n is about 20 amino acids in length, wherein at least three amino acids comprise PNG having a structure as follows (numbering according to HUMTHROM_{—}1_P22 (SEQ ID NO:51)): a sequence starting from any of amino acid numbers 490−x to 490; and ending at any of amino acid numbers 492+((n−2)−x), in which x varies from 0 to n−2.

10. An isolated polypeptide consisting of the polypeptide having a length “n”, wherein n is about 30 amino acids in length, wherein at least three amino acids comprise PNG having a structure as follows (numbering according to HUMTHROM_{—}1_P22 (SEQ ID NO:51)): a sequence starting from any of amino acid numbers 490−x to 490; and ending at any of amino acid numbers 492+((n−2)−x), in which x varies from 0 to n−2.

11. An isolated polypeptide consisting of the polypeptide having a length “n”, wherein n is about 40 amino acids in length, wherein at least three amino acids comprise PNG having a structure as follows (numbering according to HUMTHROM_{—}1_P22 (SEQ ID NO:51)): a sequence starting from any of amino acid numbers 490−x to 490; and ending at any of amino acid numbers 492+((n−2)−x), in which x varies from 0 to n−2.

12. An isolated polypeptide consisting of the polypeptide having a length “n”, wherein n is about 50 amino acids in length, wherein at least three amino acids comprise PNG having a structure as follows (numbering according to HUMTHROM_{—}1_P22 (SEQ ID NO:51)): a sequence starting from any of amino acid numbers 490−x to 490; and ending at any of amino acid numbers 492+((n−2)−x), in which x varies from 0 to n−2.

13. An antibody capable of specifically binding to an epitope of an amino acid sequence of claim 2.

14. The antibody of claim 13, wherein said antibody is capable of differentiating between a splice variant having said epitope and a corresponding known protein.

15. An antibody capable of specifically binding to an epitope of an amino acid sequence of claim 3.

16. An antibody capable of specifically binding to an epitope of an amino acid sequence of claim 4.

17. An antibody capable of specifically binding to an epitope of an amino acid sequence of claim 5.

18. An antibody capable of specifically binding to an epitope of an amino acid sequence of claim 6.

19. An antibody capable of specifically binding to an epitope of an amino acid sequence of claim 7.

20. An antibody capable of specifically binding to an epitope of an amino acid sequence of claim 8.

21. An antibody capable of specifically binding to an epitope of an amino acid sequence of claim 9.

22. An antibody capable of specifically binding to an epitope of an amino acid sequence of claim 10.

23. An antibody capable of specifically binding to an epitope of an amino acid sequence of claim 11.

24. An antibody capable of specifically binding to an epitope of an amino acid sequence of claim 12.

25. A method for treating a variant-treatable disease, comprising administering a therapeutic protein of claim 2 to a subject in need of treatment thereof.

26. A method for treating a variant-treatable disease, comprising administering an antibody of claim 13 to a subject in need of treatment thereof.

27. A nucleic acid construct comprising the isolated polynucleotide of claim 1.

28. The nucleic acid construct of claim 27, further comprising a promoter for regulating transcription of the isolated polynucleotide in sense or antisense orientation.

29. The nucleic acid construct of claim 28, further comprising positive and negative selection markers for selecting for homologous recombination events.

30. A host cell comprising the nucleic acid construct of claim 29.

31. The method of claim 25, wherein the variant-treatable disease is selected from a group consisting of cancer, such as prostate cancer, renal cancer, cervical carcinomas, breast cancer, colon cancer, colorectal cancer, pancreatic cancer, ovarian cancer, bladder cancer, lung cancer, melanoma, brain cancer, glioblastomas, soft tissue sarcomas, head-and-neck cancer, lymphomas, other tumors and tumor cell metastasis.

32. The method of claim 25, wherein the variant-treatable disease is selected from a group consisting of wound healing and inflammation, such as rheumatoid arthritis.

33. The method of claim 25, wherein the variant-treatable disease is selected from a group consisting of ocular diseases, involving treatment of retinal angiogenesis, such as diabetic rethinopathy, retinopathy of prematurity, and age-related macular degeneration.

34. A pharmaceutical composition comprising a therapeutically effective amount of a polypeptide according to claim 2 and a pharmaceutically acceptable carrier or diluent.

35. A method of treating a variant-related disease in a subject, the method comprising upregulating in the subject expression of a polypeptide of claim 2, thereby treating the variant-related disease in a subject.

36. The method of claim 35, wherein said upregulating expression of said polypeptide is effected by
i. administering said polypeptide to the subject; and/or
ii. administering an expressible polynucleotide encoding said polypeptide to the subject.


SEQUENCE LISTING (123 sequences)

SEQ ID NO: 1 - DNA, 6243 bp, Homo sapiens
agttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccagtaagtgtgaggtccgctgcaagggtgagcatgggcagcagctctgcccagctggttgcctggcatctgcagcctgcagttcagtgggtcatagagcaggaaggttacctactagagaaacaaacagaagcaaagtcctgcaggctcagcaacttcttttaatgaaaaacaaactcaccctcttccccagcattctttccatgtgtcagagaagcagaggtttcttgaacgggcttaggagagtctatgacaagggagggatttgaaagttgatcttaattgttgcctgtggttcatcttcttacagtcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttata

SEQ ID NO: 2 - DNA, 6195 bp, Homo sapiens
agttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattggtgagccacgcagcccaggatgaaacgacccaggagctttgctcttttactgaatgctgcagtcagcattcgaggagattccagcttggttagtcctgagcgatttgattgctctaagatgcaggtggacaacataatcccaacaagttatcggttccctataccctataatatcttacactgtgttaagtgcccagcatggcagtatggcagcttagaccaaccatttactgtgactgtctctctctccttgtctcagatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttata

SEQ ID NO: 3 - DNA, 6503 bp, Homo sapiens
agttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggtaaaaacagttttctatccctttttcatcttttcagttcagcaacagcctgaaacactttgggattcaaggaaattacatggctatagcaaaaaatataccaaatcaatacacaggataattagaaattattcattgtgttccagtagtttaaggatgtagatgttgccaagagaatttttaaatgagggttttgtttttcatcagaactgtttttctctgtacttgagaaattataatgcataaacaaatgccactttgttccctagattcatttcaaatgtcacatcgaaattacagtaaaattgactttgggcacactatgaactgagatgatgggattatattctacatctcactaacttctaacccacagggatccatttttttaactatgtccttttaacttttgtagtgatcgttttacactgagtgatcaattagcctatccactaggtagaaagtattgctgattttcacagttttagacatattatgcacatggtttgaggcttgagctgttttcaaggacaacattgttaagtgctccatttcttctctttgcaggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttata

SEQ ID NO: 4 - DNA, 6386 bp, Homo sapiens
agttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtacagtcaactagacgagtaaaccagaggacaggagagctgtccttgaccaaaataactgggagcgggaggaatgtaatttcatacccttcaccaaaaaaaaaagggcgaggagatgaatgtacggtctagttttagaaacgtgattagaaaatccatggtaaatcctgcaggggaaaaacagtcttccatatttaaaaatgctgctctggaataagttgtgagcagatggacttgtaaacgcctaggtgctgagcaaattcaagaaaaataaacataaagcaaagtttgcttatagcctcagggagaatggggagggacagaggtaacccacactcttccaaatggagcctctgtctactcagagatgacagggatctggattcttgtttccatgatatctgaggattctcaaaagctctgtgtaacagcagcatggtgtaccctcaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttata

SEQ ID NO: 5 - DNA, 5762 bp, Homo sapiens
agttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccaatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttata

SEQ ID NO: 6 - DNA, 224 bp, Homo sapiens
agttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacag

SEQ ID NO: 7 - DNA, 182 bp, Homo sapiens
agtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgg

SEQ ID NO: 8 - DNA, 378 bp, Homo sapiens
gcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccag

SEQ ID NO: 9 - DNA, 200 bp, Homo sapiens
ctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtg

SEQ ID NO: 10 - DNA, 127 bp, Homo sapiens
ccagcgactctgcggacgatggctggtctccatggtccgagtggacctcctgttctacgagctgtggcaatggaattcagcagcgcggccgctcctgcgatagcctcaacaaccgatgtgagggctc

SEQ ID NO: 11 - DNA, 177 bp, Homo sapiens
ttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgcccca

SEQ ID NO: 12 - DNA, 307 bp, Homo sapiens
gtaagtgtgaggtccgctgcaagggtgagcatgggcagcagctctgcccagctggttgcctggcatctgcagcctgcagttcagtgggtcatagagcaggaaggttacctactagagaaacaaacagaagcaaagtcctgcaggctcagcaacttcttttaatgaaaaacaaactcaccctcttccccagcattctttccatgtgtcagagaagcagaggtttcttgaacgggcttaggagagtctatgacaagggagggatttgaaagttgatcttaattgttgcctgtggttcatcttcttacag

SEQ ID NO: 13 - DNA, 174 bp, Homo sapiens
tcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattg

SEQ ID NO: 14 - DNA, 259 bp, Homo sapiens
gtgagccacgcagcccaggatgaaacgacccaggagctttgctcttttactgaatgctgcagtcagcattcgaggagattccagcttggttagtcctgagcgatttgattgctctaagatgcaggtggacaacataatcccaacaagttatcggttccctataccctataatatcttacactgtgttaagtgcccagcatggcagtatggcagcttagaccaaccatttactgtgactgtctctctctccttgtctcag

SEQ ID NO: 15 - DNA, 153 bp, Homo sapiens
tgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacag

SEQ ID NO: 16 - DNA, 450 bp, Homo sapiens
gtacagtcaactagacgagtaaaccagaggacaggagagctgtccttgaccaaaataactgggagcgggaggaatgtaatttcatacccttcaccaaaaaaaaaagggcgaggagatgaatgtacggtctagttttagaaacgtgattagaaaatccatggtaaatcctgcaggggaaaaacagtcttccatatttaaaaatgctgctctggaataagttgtgagcagatggacttgtaaacgcctaggtgctgagcaaattcaagaaaaataaacataaagcaaagtttgcttatagcctcagggagaatggggagggacagaggtaacccacactcttccaaatggagcctctgtctactcagagatgacagggatctggattcttgtttccatgatatctgaggattctcaaaagctctgtgtaacagcagcatggtgtaccctcag

SEQ ID NO: 17 - DNA, 168 bp, Homo sapiens
aacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaag

SEQ ID NO: 18 - DNA, 567 bp, Homo sapiens
gtaaaaacagttttctatccctttttcatcttttcagttcagcaacagcctgaaacactttgggattcaaggaaattacatggctatagcaaaaaatataccaaatcaatacacaggataattagaaattattcattgtgttccagtagtttaaggatgtagatgttgccaagagaatttttaaatgagggttttgtttttcatcagaactgtttttctctgtacttgagaaattataatgcataaacaaatgccactttgttccctagattcatttcaaatgtcacatcgaaattacagtaaaattgactttgggcacactatgaactgagatgatgggattatattctacatctcactaacttctaacccacagggatccatttttttaactatgtccttttaacttttgtagtgatcgttttacactgagtgatcaattagcctatccactaggtagaaagtattgctgattttcacagttttagacatattatgcacatggtttgaggcttgagctgttttcaaggacaacattgttaagtgctccatttcttctctttgcag

SEQ ID NO: 19 - DNA, 160 bp, Homo sapiens
gacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacg

SEQ ID NO: 20 - DNA, 235 bp, Homo sapiens
ctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacg

SEQ ID NO: 21 - DNA, 145 bp, Homo sapiens
gcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatc

SEQ ID NO: 22 - DNA, 255 bp, Homo sapiens
gttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacag

SEQ ID NO: 23 - DNA, 140 bp, Homo sapiens
agtggtgatgtatgaagggaagaaaatcatggctgactcaggacccatctatgataaaacctatgctggtggtagactagggttgtttgtcttctctcaagaaatggtgttcttctctgacctgaaatacgaatgtagag

SEQ ID NO: 24 - DNA, 3807 bp, Homo sapiens
atccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttatacttactgtatccatgcttatttgttctctactggctttatgtcatgaagtatatgcgtaaataccattcataaatcaatatagcatatacaaaaataaattacagtaagtcatagcaacattcacagtttgtatgtgattgagaaagactgagttgctcaggcctaggcttagaatttgctgcgtttgtggaataaaagaacaaaatgatacattagcctgccatatcaaaaacatataaaagagaaattatccctaagtcaagggcccccataagaataaaatttcttattaaggtcattagatgtcattgaatccttttcaaagtgcagtatgaaaacaaagggaaaaacactgaagcacacgcaactctcacagcgacattttctgacccacgaatgatgccttgggtgggcaacacgattgcatgttgtggagacacttcggaagtaaatgtggatgagggaggagctgtccttgcaatgttgagccaagcattacagatacctcctcttgaagaaggaataataagtttaatcaaaaaagaagactaaaaaatgtaaaatttggaaggaatccataaatgcgtgtgtgtctaaatacaaattatcatgtgaagaaaaggcccaagtgtaccaataagcagaccttgatttttggatgggctaattatgaatgtggaatactgaccagttaatttccagttttaatgaaaacagatcaaagaagaaattttatgagtaggttaaaggtctggctttgaggtctattaaacactagaaaggactggctgggtgagataaaatcttccttgttgattttcactctcattctataaatactcatctttctgagtagccatgatcacatacaaatgtaaattgccaaatcattttatagtaccaaggtgaagaagcaggaactagaaagtgttgataatagctgtggagttaggaaaactgatgtgaaggaaataattctttgaaatggcaaagaattaaataccatcattcattatcagaagagttcaacgtttgaagtgctgggagataattctaattcattcttggatagtgaagcaaaactgattgaaaataccaagataagacagaaaaagtgactggaaagaggagcttttcttccaggcatgttccagtttcaccctaagactgaccttcaaataatcaggttgtactgaaataaaggacttgttaaaaattaaaattatgtcatcgagatgatagcttttttcctcctccaacagtttattgtcatgtgttgtgggagagctcgagtgaagagcaataaactccaggtcttataagaatgtacatacaataaaggtggtgccagcagtttttttttttctaaagagtcacatgtagaaaagcctccagtattaagctcctgaattcattccttaaataaattggctctctctctcttctataatttctttttctttttatttttgagatgaagtcttgctctgtcgcccaggctggagtgcagtgacacaatctcggctcactgcaacctctgcctccccggttcaagcaattctccctcctgcctcagcctcccaagtagctgggactacaagcgcccgccaccaagcctggctaattctgtatttttagtaaagacggggtttcaccttgttccggacaaacactaagccctaaagggaaatccaaaataa

SEQ ID NO: 25 - DNA, 32 bp, Homo sapiens
gcattccccgcgcccctccagccctcgccgcc

SEQ ID NO: 26 - DNA, 40 bp, Homo sapiens
ctcgccaccgctcccggccgccgcgctccggtacacacag

SEQ ID NO: 27 - DNA, 96 bp, Homo sapiens
gatccctgctgggcaccaacagctccaccatggggctggcctggggactaggcgtcctgttcctgatgcatgtgtgtggcaccaaccgcattccag

SEQ ID NO: 28 - DNA, 76 bp, Homo sapiens
ggggtgctgcagaatgtgaggtttgtctttggaaccacaccagaagacatcctcaggaacaaaggctgctccagct

SEQ ID NO: 29 - DNA, 84 bp, Homo sapiens
actgaagagaacaaagagttggccaatgagctgaggcggcctcccctatgctatcacaacggagttcagtacagaaataacgag

SEQ ID NO: 30 - DNA, 39 bp, Homo sapiens
gaatggactgttgatagctgcactgagtgtcactgtcag

SEQ ID NO: 31 - DNA, 94 bp, Homo sapiens
aactcagttaccatctgcaaaaaggtgtcctgccccatcatgccctgctccaatgccacagttcctgatggagaatgctgtcctcgctgttggc

SEQ ID NO: 32 - DNA, 47 bp, Homo sapiens
ctcggtccagacacggacctgccacattcaggagtgtgacaagagat

SEQ ID NO: 33 - DNA, 39 bp, Homo sapiens
atggatgcctgtccaatccctgctttgccggcgtgaagt

SEQ ID NO: 34 - DNA, 35 bp, Homo sapiens
gtactagctaccctgatggcagctggaaatgtggt

SEQ ID NO: 35 - DNA, 54 bp, Homo sapiens
gcttgtccccctggttacagtggaaatggcatccagtgcacagatgttgatgag

SEQ ID NO: 36 - DNA, 51 bp, Homo sapiens
gtgtgcaagccccgtaacccctgcacggatgggacccacgactgcaacaag

SEQ ID NO: 37 - DNA, 108 bp, Homo sapiens
gataattgccccaaccttcccaactcagggcaggaagactatgacaaggatggaattggtgatgcctgtgatgatgacgatgacaatgataaaattccagatgacagg

SEQ ID NO: 38 - DNA, 119 bp, Homo sapiens
gtatcctcaatgaacgggacaactgccagtacgtctacaatgtggaccagagagacactgatatggatggggttggagatcagtgtgacaattgccccttggaacacaatccggatcag

SEQ ID NO: 39 - DNA, 54 bp, Homo sapiens
ccaaaatgaccctaactgggttgtacgccatcagggtaaagaactcgtccagac

SEQ ID NO: 40 - DNA, 29 bp, Homo sapiens
tgtcaactgtgatcctggactcgctgtag

SEQ ID NO: 41 - DNA, 17 bp, Homo sapiens
gaaacacccctggccag

SEQ ID NO: 42 - DNA, 34 bp, Homo sapiens
gtgcgcaccctgtggcatgaccctcgtcacatag

SEQ ID NO: 43 - DNA, 64 bp, Homo sapiens
gctggaaagatttcaccgcctacagatggcgtctcagccacaggccaaagacgggtttcattag

SEQ ID NO: 44 - protein, 1170 aa, Homo sapiens [sequence omitted]

SEQ ID NO: 45 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 46 - protein, 59 aa, Homo sapiens
PDGECCPRCWPSDSADDGWSPWSEWTSCSTSCGNGIQQRGRSCDSLNNRCEGSSVQTRT

SEQ ID NO: 47 - protein, 1170 aa, Homo sapiens; identical to SEQ ID NO: 45

SEQ ID NO: 48 - protein, 578 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPSKCEVRCKGEHGQQLCPAGCLASAACSSVGHRAGRLPTRETNRSKVLQAQQLLLMKNKLTLFPSILSMCQRSRGFLNGLRRVYDKGGI

SEQ ID NO: 49 - protein, 804 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRVKTVFYPFFIFSVQQQPETLWDSRKLHGYSKKYTKSIHRIIRNYSLCSSSLRM

SEQ ID NO: 50 - protein, 685 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVQSTRRVNQRTGELSLTKITGSGRNVISYPSPKKKGRGDECTV

SEQ ID NO: 51 - protein, 1112 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPNGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 52 - protein, 555 aa, Homo sapiens [sequence omitted]

SEQ ID NO: 53 - DNA, 3599 bp, Homo sapiens
gaattcgccaccatgggcctggcctggggtttgggagtgctgtttctcatgcatgtttgcgggactaacaggatccctgaaagcgggggagacaactctgtgtttgatatttttgagctgaccggggcagcccgcaaggggagtggacggaggctcgtgaagggccctgatcctagcagtccagccttccgcattgaggacgccaatcttattccacccgtgccggatgataagttccaggacctcgtagacgccgtgcgcgcggagaagggattcctccttctcgctagtctgcgccaaatgaaaaaaaccagggggaccctcctggcacttgagaggaaggaccattccgggcaagtctttagtgtggtctcaaatggaaaggcaggcactctcgacctttccctcacagttcaaggcaagcaacacgtggtgtcagtggaggaggctctgctggccacagggcagtggaaatccatcaccctgtttgttcaggaggacagggcacagctgtacattgactgtgagaagatggaaaatgcggagctcgacgtgccaatccagtcagtattcacacgagacctggctagcattgcccggctcaggatagccaagggcggagttaacgacaactttcaaggcgtgcttcagaacgtccgatttgtgtttggaacaacacccgaggatattttgaggaataagggatgcagctcctccacctccgtcctgttgactcttgataataatgtggtcaatggttcctccccagcaatccgcacaaactatatcggccacaagacaaaagacctccaggccatctgcggtatcagttgcgacgagctgagcagcatggtcctcgaattgcgcgggctgaggaccatcgtcactactctgcaggattccatcaggaaggtaaccgaagagaataaagaactggctaacgaactgcgcagacctcctctgtgctatcataatggtgtccaatataggaacaacgaagagtggaccgttgatagttgtaccgaatgtcattgccagaacagcgtaaccatatgcaaaaaggtcagttgtcccattatgccttgcagcaatgcaactgtgccagatggggaatgctgcccacgatgctggccaagtgactcagccgatgatgggtggtcaccatggagcgagtggacgtcctgtagtacgtcttgtggcaacggcattcagcagcgaggacgcagttgtgattctctcaataatcgatgcgagggcagcagcgtgcagacccggacatgtcatattcaggagtgtgacaagaggttcaagcaggatggtggctggagccattggtccccatggtctagttgttcagtgacctgcggtgacggagttatcacacgaatccgcctgtgcaactcccctagcccacagatgaatggaaagccatgtgagggggaggccagggaaacaaaggcttgtaagaaagacgcatgtcctatcaatggagggtggggcccttggagcccctgggatatttgttccgtgacatgcggcgggggagtacagaaaaggagtagactttgcaataaccccactccgcaatttgggggtaaagactgcgtcggagacgtaacagaaaatcagatctgtaataaacaggactgccccattgacgggtgcctgagcaacccttgttttgcaggggtgaaatgcactagttatcctgatggctcatggaaatgcggtgcatgtccccccggatatagcggcaacggcattcagtgcacggatgtagacgaatgcaaagaagtcccagacgcgtgcttcaaccataacggcgagcataggtgcgagaacaccgaccccggctataattgcttgccctgcccaccacgcttcaccgggtcccagccctttggccagggcgtagagcatgcgaccgccaacaagcaggtgtgcaaacctcgcaatccttgtaccgacggcacacatgattgtaacaagaacgcaaaatgcaattacttgggccactacagtgaccccatgtatcggtgcgagtgcaaaccgggctacgcagggaacggtatcatttgcggtgaggatactgatctggacggctggccaaacgaaaatctcgtttgcgtggccaacgctacctaccattgtaaaaaggataattgccccaatctccctaattccggacaagaggattacgacaaggatgggatcggggatgcgtgcgacgacgatgatgacaatgacaagattccggacgaccgcgataattgtcccttccattacaatccagcacaatacgactatgatcgagacgatgtcggggatagatgtgacaactgcccgtataatcataatccagatcaagccgacacggacaacaacggcgaaggcgacgcctgtgccgccgatattgacggagacgggatactgaatgagcgggacaactgtcaatacgtgtacaatgtggaccagcgggacacagatatggatggcgtgggcgatcaatgtgataattgtccactcgagcacaacccggaccagctcgacagtgactctgatcgaattggcgacacatgtgacaacaatcaggacattgacgaggacggccaccagaacaacctcgacaattgcccgtacgttcccaacgcgaaccaggctgatcacgacaaagacggcaaaggcgatgcgtgcgaccacgacgatgataacgatggcatccctgacgacaaggataattgccggttggtcccaaacccagaccagaaagactcagacggggacggacgcggagatgcctgcaaggatgactttgaccatgacagcgttccggatatcgatgacatttgtccagagaatgttgatatcagtgagaccgacttccgccggtttcagatgatacccctggaccctaaaggcacttctcagaatgacccaaattgggtagtacggcaccaaggcaaggagcttgtgcaaaccgtcaactgcgaccccggactcgctgtgggatatgacgagttcaacgccgtggacttctccggaactttcttcataaacaccgagcgggacgatgactacgcaggcttcgtgttcggttaccaaagctctagcaggttctacgtggtgatgtggaagcaagttacccagtcatactgggacactaatccgacgcgcgcacaggggtattccggtctttctgttaaggtcgtgaactccactaccgggccgggagagcacctcaggaatgcactgtggcacacaggaaatactccaggacaggtgaggactctttggcatgatcctagacacattggatggaaagacttcacagcttatagatggaggctcagccatcgacccaaaaccggattcattagagttgtgatgtatgaaggtaaaaaaatcatggctgattctggccccatctacgataagacatatgcaggcggacggctggggctgttcgtattctcccaggagatggtattcttttcagacctgaagtatgagtgtcgcgatccgtggagccatccccaattcgaaaaaaccggacaccatcaccaccaccaccaccacggcggccagtgataggcggccgc

SEQ ID NO: 54 - protein, 1191 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDPWSHPQFEKTGHHHHHHHHGGQ

SEQ ID NO: 55 - DNA, 3425 bp, Homo sapiens
gaattcgccaccatgggcctggcctggggtttgggagtgctgtttctcatgcatgtttgcgggactaacaggatccctgaaagcgggggagacaactctgtgtttgatatttttgagctgaccggggcagcccgcaaggggagtggacggaggctcgtgaagggccctgatcctagcagtccagccttccgcattgaggacgccaatcttattccacccgtgccggatgataagttccaggacctcgtagacgccgtgcgcgcggagaagggattcctccttctcgctagtctgcgccaaatgaaaaaaaccagggggaccctcctggcacttgagaggaaggaccattccgggcaagtctttagtgtggtctcaaatggaaaggcaggcactctcgacctttccctcacagttcaaggcaagcaacacgtggtgtcagtggaggaggctctgctggccacagggcagtggaaatccatcaccctgtttgttcaggaggacagggcacagctgtacattgactgtgagaagatggaaaatgcggagctcgacgtgccaatccagtcagtattcacacgagacctggctagcattgcccggctcaggatagccaagggcggagttaacgacaactttcaaggcgtgcttcagaacgtccgatttgtgtttggaacaacacccgaggatattttgaggaataagggatgcagctcctccacctccgtcctgttgactcttgataataatgtggtcaatggttcctccccagcaatccgcacaaactatatcggccacaagacaaaagacctccaggccatctgcggtatcagttgcgacgagctgagcagcatggtcctcgaattgcgcgggctgaggaccatcgtcactactctgcaggattccatcaggaaggtaaccgaagagaataaagaactggctaacgaactgcgcagacctcctctgtgctatcataatggtgtccaatataggaacaacgaagagtggaccgttgatagttgtaccgaatgtcattgccagaacagcgtaaccatatgcaaaaaggtcagttgtcccattatgccttgcagcaatgcaactgtgccagatggggaatgctgcccacgatgctggccaagtgactcagccgatgatgggtggtcaccatggagcgagtggacgtcctgtagtacgtcttgtggcaacggcattcagcagcgaggacgcagttgtgattctctcaataatcgatgcgagggcagcagcgtgcagacccggacatgtcatattcaggagtgtgacaagaggttcaagcaggatggtggctggagccattggtccccatggtctagttgttcagtgacctgcggtgacggagttatcacacgaatccgcctgtgcaactcccctagcccacagatgaatggaaagccatgtgagggggaggccagggaaacaaaggcttgtaagaaagacgcatgtcctaatgggtgcctgagcaacccttgttttgcaggggtgaaatgcactagttatcctgatggctcatggaaatgcggtgcatgtccccccggatatagcggcaacggcattcagtgcacggatgtagacgaatgcaaagaagtcccagacgcgtgcttcaaccataacggcgagcataggtgcgagaacaccgaccccggctataattgcttgccctgcccaccacgcttcaccgggtcccagccctttggccagggcgtagagcatgcgaccgccaacaagcaggtgtgcaaacctcgcaatccttgtaccgacggcacacatgattgtaacaagaacgcaaaatgcaattacttgggccactacagtgaccccatgtatcggtgcgagtgcaaaccgggctacgcagggaacggtatcatttgcggtgaggatactgatctggacggctggccaaacgaaaatctcgtttgcgtggccaacgctacctaccattgtaaaaaggataattgccccaatctccctaattccggacaagaggattacgacaaggatgggatcggggatgcgtgcgacgacgatgatgacaatgacaagattccggacgaccgcgataattgtcccttccattacaatccagcacaatacgactatgatcgagacgatgtcggggatagatgtgacaactgcccgtataatcataatccagatcaagccgacacggacaacaacggcgaaggcgacgcctgtgccgccgatattgacggagacgggatactgaatgagcgggacaactgtcaatacgtgtacaatgtggaccagcgggacacagatatggatggcgtgggcgatcaatgtgataattgtccactcgagcacaacccggaccagctcgacagtgactctgatcgaattggcgacacatgtgacaacaatcaggacattgacgaggacggccaccagaacaacctcgacaattgcccgtacgttcccaacgcgaaccaggctgatcacgacaaagacggcaaaggcgatgcgtgcgaccacgacgatgataacgatggcatccctgacgacaaggataattgccggttggtcccaaacccagaccagaaagactcagacggggacggacgcggagatgcctgcaaggatgactttgaccatgacagcgttccggatatcgatgacatttgtccagagaatgttgatatcagtgagaccgacttccgccggtttcagatgatacccctggaccctaaaggcacttctcagaatgacccaaattgggtagtacggcaccaaggcaaggagcttgtgcaaaccgtcaactgcgaccccggactcgctgtgggatatgacgagttcaacgccgtggacttctccggaactttcttcataaacaccgagcgggacgatgactacgcaggcttcgtgttcggttaccaaagctctagcaggttctacgtggtgatgtggaagcaagttacccagtcatactgggacactaatccgacgcgcgcacaggggtattccggtctttctgttaaggtcgtgaactccactaccgggccgggagagcacctcaggaatgcactgtggcacacaggaaatactccaggacaggtgaggactctttggcatgatcctagacacattggatggaaagacttcacagcttatagatggaggctcagccatcgacccaaaaccggattcattagagttgtgatgtatgaaggtaaaaaaatcatggctgattctggccccatctacgataagacatatgcaggcggacggctggggctgttcgtattctcccaggagatggtattcttttcagacctgaagtatgagtgtcgcgatccgtggagccatccccaattcgaaaaaaccggacaccatcaccaccaccaccaccacggcggccagtgataggcggccgc

SEQ ID NO: 56 - protein, 1133 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPNGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDPWSHPQFEKTGHHHHHHHHGGQ

SEQ ID NO: 57 - DNA, 2147 bp, Homo sapiens
gaattcgccaccatgggcctggcctggggtttgggagtgctgtttctcatgcatgtttgcgggactaacaggatccctgaaagcgggggagacaactctgtgtttgatatttttgagctgaccggggcagcccgcaaggggagtggacggaggctcgtgaagggccctgatcctagcagtccagccttccgcattgaggacgccaatcttattccacccgtgccggatgataagttccaggacctcgtagacgccgtgcgcgcggagaagggattcctccttctcgctagtctgcgccaaatgaaaaaaaccagggggaccctcctggcacttgagaggaaggaccattccgggcaagtctttagtgtggtctcaaatggaaaggcaggcactctcgacctttccctcacagttcaaggcaagcaacacgtggtgtcagtggaggaggctctgctggccacagggcagtggaaatccatcaccctgtttgttcaggaggacagggcacagctgtacattgactgtgagaagatggaaaatgcggagctcgacgtgccaatccagtcagtattcacacgagacctggctagcattgcccggctcaggatagccaagggcggagttaacgacaactttcaaggcgtgcttcagaacgtccgatttgtgtttggaacaacacccgaggatattttgaggaataagggatgcagctcctccacctccgtcctgttgactcttgataataatgtggtcaatggttcctccccagcaatccgcacaaactatatcggccacaagacaaaagacctccaggccatctgcggtatcagttgcgacgagctgagcagcatggtcctcgaattgcgcgggctgaggaccatcgtcactactctgcaggattccatcaggaaggtaaccgaagagaataaagaactggctaacgaactgcgcagacctcctctgtgctatcataatggtgtccaatataggaacaacgaagagtggaccgttgatagttgtaccgaatgtcattgccagaacagcgtaaccatatgcaaaaaggtcagttgtcccattatgccttgcagcaatgcaactgtgccagatggggaatgctgcccacgatgctggccaagtgactcagccgatgatgggtggtcaccatggagcgagtggacgtcctgtagtacgtcttgtggcaacggcattcagcagcgaggacgcagttgtgattctctcaataatcgatgcgagggcagcagcgtgcagacccggacatgtcatattcaggagtgtgacaagaggttcaagcaggatggtggctggagccattggtccccatggtctagttgttcagtgacctgcggtgacggagttatcacacgaatccgcctgtgcaactcccctagcccacagatgaatggaaagccatgtgagggggaggccagggaaacaaaggcttgtaagaaagacgcatgtcctatcaatggagggtggggcccttggagcccctgggatatttgttccgtgacatgcggcgggggagtacagaaaaggagtagactttgcaataaccccactccgcaatttgggggtaaagactgcgtcggagacgtaacagaaaatcagatctgtaataaacaggactgccccattgacgggtgcctgagcaacccttgttttgcaggggtgaaatgcactagttatcctgatggctcatggaaatgcggtgcatgtccccccggatatagcggcaacggcattcagtgcacggatgtagacgaatgcaaagaagtcccagacgcgtgcttcaaccataacggcgagcataggtgcgagaacaccgaccccggctataattgcttgccctgcccaccacgcttcaccgggtcccagccctttggccagggcgtagagcatgcgaccgccaacaagcaggtgcagtccactcgccgcgtgaaccagagaactggagagttgtcactgactaagatcacaggctctggtaggaacgtcatctcctatccatccccaaagaagaagggaaggggtgatgaatgcaccgtaccgtggagccatccccaattcgaaaaaaccggacaccatcaccaccaccaccaccacggcggccagtgataggcggccgc

SEQ ID NO: 58 - protein, 707 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVQSTRRVNQRTGELSLTKITGSGRNVISYPSPKKKGRGDECTVPWSHPQFEKTGHHHHHHHHGGQ

SEQ ID NO: 59 - DNA, 1757 bp, Homo sapiens
gaattcgccaccatgggcctggcctggggtttgggagtgctgtttctcatgcatgtttgcgggactaacaggatccctgaaagcgggggagacaactctgtgtttgatatttttgagctgaccggggcagcccgcaaggggagtggacggaggctcgtgaagggccctgatcctagcagtccagccttccgcattgaggacgccaatcttattccacccgtgccggatgataagttccaggacctcgtagacgccgtgcgcgcggagaagggattcctccttctcgctagtctgcgccaaatgaaaaaaaccagggggaccctcctggcacttgagaggaaggaccattccgggcaagtctttagtgtggtctcaaatggaaaggcaggcactctcgacctttccctcacagttcaaggcaagcaacacgtggtgtcagtggaggaggctctgctggccacagggcagtggaaatccatcaccctgtttgttcaggaggacagggcacagctgtacattgactgtgagaagatggaaaatgcggagctcgacgtgccaatccagtcagtattcacacgagacctggctagcattgcccggctcaggatagccaagggcggagttaacgacaactttcaaggcgtgcttcagaacgtccgatttgtgtttggaacaacacccgaggatattttgaggaataagggatgcagctcctccacctccgtcctgttgactcttgataataatgtggtcaatggttcctccccagcaatccgcacaaactatatcggccacaagacaaaagacctccaggccatctgcggtatcagttgcgacgagctgagcagcatggtcctcgaattgcgcgggctgaggaccatcgtcactactctgcaggattccatcaggaaggtaaccgaagagaataaagaactggctaacgaactgcgcagacctcctctgtgctatcataatggtgtccaatataggaacaacgaagagtggaccgttgatagttgtaccgaatgtcattgccagaacagcgtaaccatatgcaaaaaggtcagttgtcccattatgccttgcagcaatgcaactgtgccagatggggaatgctgcccacgatgctggccaagtgactcagccgatgatgggtggtcaccatggagcgagtggacgtcctgtagtacgtcttgtggcaacggcattcagcagcgaggacgcagttgtgattctctcaataatcgatgcgagggcagcagcgtgcagacccggacatgtcatattcaggagtgtgacaagaggttcaagcaggatggtggctggagccattggtccccatggtctagttgttcagtgacctgcggtgacggagttatcacacgaatccgcctgtgcaactcccctagcccacagatgaatggaaagccatgtgagggggaggccagggaaacaaaggcttgtaagaaagacgcatgtcctatcaatggagggtggggcccttggagcccctgggatatttgttccgtgacatgcggcgggggagtacagaaaaggagtagactttgcaataaccccactccgcaatttgggggtaaagactgcgtcggagacgtaacagaaaatcagatctgtaataaacaggactgccccattggtgaaccccggtctcccgggccgtggagccatccccaattcgaaaaaaccggacaccatcaccaccaccaccaccacggcggccagtgataggcggccgc

SEQ ID NO: 60 - protein, 577 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIGEPRSPGPWSHPQFEKTGHHHHHHHHGGQ

SEQ ID NO: 61 - DNA, 713 bp, Homo sapiens
gaattcgccaccatgaactccttctctacatccgctttcgggccggtagcgttctctctgggcttgctcctggtgctgcctgctgcctttcccgccccagttccacccggcgatgattccgcagatgacggatggagtccatggagcgagtggacctcatgctccaccagctgtggcaacgggatccaacagaggggcaggagctgtgattctctcaacaacaggtgtgaaggatcttccgtacagactcggacctgtcacattcaggagtgcgacaagcgctttaaacaggatggcggctggtctcactggtcaccctggtcaagttgtagcgtgacttgtggcgacggtgtcattacccggattaggctctgtaacagtccatctccacaaatgaacggcaagccctgcgaaggagaagccagagagacaaaagcgtgcaagaaggatgcttgcccaatcaacggaggttggggcccatggagcccgtgggatatctgtagtgtgacatgcgggggcggggtgcagaagcggtccaggctgtgtaacaatcccaccccgcagttcgggggaaaagattgcgtcggggatgtgacggaaaaccagatctgtaataagcaggactgtcccattccttggtctcatccccagttcgaaaagaccgggcatcatcaccaccaccaccaccacggggggcagtgataagcggccgc

SEQ ID NO: 62 - protein, 229 aa, Homo sapiens
MNSFSTSAFGPVAFSLGLLLVLPAAFPAPVPPGDDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIPWSHPQFEKTGHHHHHHHHGGQ

SEQ ID NO: 63 - DNA, 20 bp, Homo sapiens
gctcctgcgatagcctcaac

SEQ ID NO: 64 - DNA, 20 bp, Homo sapiens
caaatcgctcaggactaacc

SEQ ID NO: 65 - DNA, 25 bp, Homo sapiens
aaccacaccagaagacatcctcagg

SEQ ID NO: 66 - DNA, 25 bp, Homo sapiens
ccatccgcactaactacattggcca

SEQ ID NO: 67 - DNA, 25 bp, Homo sapiens
caaaggacttgcaagccatctgcgg

SEQ ID NO: 68 - DNA, 25 bp, Homo sapiens
gcggcatctcctgtgatgagctgtc

SEQ ID NO: 69 - DNA, 25 bp, Homo sapiens
agcatggtcctggaactcaggggcc

SEQ ID NO: 70 - DNA, 25 bp, Homo sapiens
cattgtgaccacgctgcaggacagc

SEQ ID NO: 71 - DNA, 25 bp, Homo sapiens
tggccaatgagctgaggcggcctcc

SEQ ID NO: 72 - DNA, 25 bp, Homo sapiens
cccctatgctatcacaacggagttc

SEQ ID NO: 73 - DNA, 25 bp, Homo sapiens
atggactgttgatagctgcactgag

SEQ ID NO: 74 - DNA, 25 bp, Homo sapiens
tgatggagaatgctgtcctcgctgt

SEQ ID NO: 75 - DNA, 25 bp, Homo sapiens
ccagcgactctgcggacgatggctg

SEQ ID NO: 76 - DNA, 25 bp, Homo sapiens
tgaccctcgtcacataggctggaaa

SEQ ID NO: 77 - DNA, 25 bp, Homo sapiens
gaaagatttcaccgcctacagatgg

SEQ ID NO: 78 - DNA, 25 bp, Homo sapiens
tacagatggcgtctcagccacaggc

SEQ ID NO: 79 - DNA, 25 bp, Homo sapiens
gactagggttgtttgtcttctctca

SEQ ID NO: 80 - DNA, 25 bp, Homo sapiens
agaaatggtgttcttctctgacctg

SEQ ID NO: 81 - DNA, 25 bp, Homo sapiens
accaatgctggtattgcaccttctg

SEQ ID NO: 82 - DNA, 25 bp, Homo sapiens
gcaccttctggaactatgggcttga

SEQ ID NO: 83 - DNA, 25 bp, Homo sapiens
gagaaaacccccaggatcacttctc

SEQ ID NO: 84 - DNA, 25 bp, Homo sapiens
ccttcttttctgtgcttgcatcagt

SEQ ID NO: 85 - DNA, 25 bp, Homo sapiens
cagtgtggactcctagaacgtgcga

SEQ ID NO: 86 - DNA, 25 bp, Homo sapiens
aacagactcatcagcattcagcctc

SEQ ID NO: 87 - DNA, 25 bp, Homo sapiens
tcatgatgctgactggcgttagctg

SEQ ID NO: 88 - DNA, 25 bp, Homo sapiens
ggcgttagctgattaacccatgtaa

SEQ ID NO: 89 - DNA, 25 bp, Homo sapiens
gacaaagactggcttctggacttcc

SEQ ID NO: 90 - DNA, 25 bp, Homo sapiens
tgccattgcctggtcacattgaaat

SEQ ID NO: 91 - DNA, 25 bp, Homo sapiens
ggtggcttcattctagatgtagctt

SEQ ID NO: 92 - DNA, 25 bp, Homo sapiens
taccatctcagtgagcaccagctgc

SEQ ID NO: 93 - DNA, 25 bp, Homo sapiens
accagctgcctcccaaaggaggggc

SEQ ID NO: 94 - DNA, 25 bp, Homo sapiens
aggggcagccgtgcttatattttta

SEQ ID NO: 95 - DNA, 25 bp, Homo sapiens
tatcaacctaactaaaacattcctt

SEQ ID NO: 96 - DNA, 25 bp, Homo sapiens
gcgtaaagactatccatgtcatctt

SEQ ID NO: 97 - DNA, 25 bp, Homo sapiens
atctttgttgagagtcttcgtgact

SEQ ID NO: 98 - DNA, 25 bp, Homo sapiens
aacttacatacaaatattacctcat

SEQ ID NO: 99 - DNA, 25 bp, Homo sapiens
attacctcatttgttgtgtgactga

SEQ ID NO: 100 - DNA, 25 bp, Homo sapiens
agtgtctaacaaacttaaagctact

SEQ ID NO: 101 - DNA, 25 bp, Homo sapiens
aagtcagtgttgtacatagcataaa

SEQ ID NO: 102 - DNA, 25 bp, Homo sapiens
tatcatctggtataccattgcttta

SEQ ID NO: 103 - DNA, 25 bp, Homo sapiens
tttctcattgccattggaatagaat

SEQ ID NO: 104 - DNA, 25 bp, Homo sapiens
ttatcaggaaatactgcctgtagag

SEQ ID NO: 105 - DNA, 25 bp, Homo sapiens
gcctgtagagttagtatttctattt

SEQ ID NO: 106 - DNA, 25 bp, Homo sapiens
aatgtttgcacactgaattgaagaa

SEQ ID NO: 107 - DNA, 25 bp, Homo sapiens
ctatttgccaatacctttttctagg

SEQ ID NO: 108 - DNA, 25 bp, Homo sapiens
gtgtaagttgtatattactgtttct

SEQ ID NO: 109 - DNA, 25 bp, Homo sapiens
attgttccatagcacgttattcctg

SEQ ID NO: 110 - DNA, 25 bp, Homo sapiens
gcacgttattcctggcttttgttac

SEQ ID NO: 111 - DNA, 25 bp, Homo sapiens
acacccttgtcacagctcagaataa

SEQ ID NO: 112 - DNA, 25 bp, Homo sapiens
gaataaccaattccatccagggatc

SEQ ID NO: 113 - DNA, 25 bp, Homo sapiens
gcgatattggcactgtaatggtcgt

SEQ ID NO: 114 - DNA, 25 bp, Homo sapiens
atttatgttctgttccgcattcact

SEQ ID NO: 115 - DNA, 25 bp, Homo sapiens
tgttccgcattcacttaacatgtgc

SEQ ID NO: 116 - DNA, 25 bp, Homo sapiens
tagatgtgattgtagccgtggtgcc

SEQ ID NO: 117 - DNA, 25 bp, Homo sapiens
agccgtggtgcctgggcagatggta

SEQ ID NO: 118 - DNA, 25 bp, Homo sapiens
aaacatgctgtcctcttatgacaat

SEQ ID NO: 119 - DNA, 25 bp, Homo sapiens
atgtgcagagaaggccccaaacgct

SEQ ID NO: 120 - DNA, 20 bp, Homo sapiens
tgatagctgcactgagtgtc

SEQ ID NO: 121 - DNA, 20 bp, Homo sapiens
ctctatgacccactgaactg

SEQ ID NO: 122 - DNA, 542 bp, Homo sapiens
gctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattggtgagccacgcagcccaggatgaaacgacccaggagctttgctcttttactgaatgctgcagtcagcattcgaggagattccagcttggttagtcctgagcgatttg

SEQ ID NO: 123 - DNA, 569 bp, Homo sapiens
tgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccagtaagtgtgaggtccgctgcaagggtgagcatgggcagcagctctgcccagctggttgcctggcatctgcagcctgcagttcagtgggtcatagag